SEQUENCE LISTING (191 sequences)

SEQ ID NO: 1 - protein, 145 aa, Homo sapiens
MSWAPVLLMLFVYCTGCGPQPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSEKEEREREWEEEMEPTAARTRVP

SEQ ID NO: 2 - protein, 142 aa, Homo sapiens
MAWTSVLLMLLAYLTGCGPQPMVHQPPLASSSLGATIRLSCTLSNDHNIGIYSIYWYQQRPGHPPRFLLRYFSHSDKHQGPDIPPRFSGSKDTTRNLGYLSISELQPEDEAVYYCAVGLRSQEKKRMEREWEGEKSYTDLGS

SEQ ID NO: 3 - protein, 213 aa, Homo sapiens
MRPGTGQGGLEAPGEPGPNLRQRWPLLLLGLAVVTHGLLRPTAASQSRALGPGAPGGSSRSSLRSRWGRFLLQRGSWTGPRCWPRGFQSKHNSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 4 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 115 aa, Artificial Sequence
  Synthetic peptide
QVQLQQPGAELVKPGASVKLSCKASGYTFTSYWMQWVKQRPGQGLEWIGEINPSNGRINYNEKFKSKATLTVDISSSTAYMQLSSLTSEDSAVYYCARSGLLDYWGQGTTLTDSS

SEQ ID NO: 7 - protein, 115 aa, Artificial Sequence
  Synthetic peptide
EVQLQQSGAELVKPGASVQLSCKASGYTFTSYWMQWVKQRPGQGLEWIGEINPSNGRNNYNEKFKRKATLTVDTSSSTAYMQLSSLTSEDSAVYYCARSGLLDYWGQGTTLTVSS

SEQ ID NO: 8 - protein, 118 aa, Artificial Sequence
  Synthetic peptide
EVQLEESGPSLVKPSQTLSLTCSVTGDSITSDYWTWIRKFPGNKLEYMGYISYSGRTYYNPSLKSRISITRDTSKKQYYLQLNSVTTEDTATYYCARERYYYGSLDYWGQGTTLTVSS

SEQ ID NO: 9 - protein, 118 aa, Artificial Sequence
  Synthetic peptide
EVQLEESGPSLVKPSQTLSLTCSVTGDSITSDYWTWIRKFPGNKLEYMGYISYSGRTYYNPSLKSRISITRDTSRNQYYLQLSSVTTEDTATYYCARERYYYGSLDYWGQGTTLTVSS

SEQ ID NO: 10 - protein, 118 aa, Artificial Sequence
  Synthetic peptide
EVQLEESGPSLVKPSQTLSLTCSVTGDSITSDYWTWIRKFPGNILEYMGYISSSGRIYYNPSLKSRISITRDTSKNQYYLQLSSVTTEDTATYYCARERYYYGSLDYWGQGTTLTVSS

SEQ ID NO: 11 - protein, 115 aa, Artificial Sequence
  Synthetic peptide
QVQLQQPGAELVKPGASVKLSCKASGYTFTSNWMNWVKQRPGQGLEWIGEINPSNGRINYNEKFKSKATLTVDKSSSTAYMQLSSLTSEDSAVYYCARSGLLDYWGQGTTLTVSS

SEQ ID NO: 12 - protein, 113 aa, Artificial Sequence
  Synthetic peptide
DVLMTQTPLSLPVSLGDQASISCRSSQSLIHSNGNTYLHWSLQKPGQSPKLLIYKVSNRFSGVPDRFSGSGSGTDFTLKISSVEAEDLGVYFCSQSTYVPLTFGAGTKLELKR

SEQ ID NO: 13 - protein, 113 aa, Artificial Sequence
  Synthetic peptide
DVLMTQTPLSLPVSLGDQASISCRSSQSLVHSNGNTYLHWYLQKPGQSPKLLIYKVSNRFSGVPDRFSGSGSGTDFTLTISRVEAEDLGVYFCSQSTYVPLTFGAGTKLELKR

SEQ ID NO: 14 - protein, 113 aa, Artificial Sequence
  Synthetic peptide
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQTTHVPPTFGGGTKLEIKR

SEQ ID NO: 15 - protein, 113 aa, Artificial Sequence
  Synthetic peptide
DVLMTQTPLSLPVSLGDQASISCRSSQSLVHSNGNTYLHWYLQKPGQSPKLLIYKVSNRFSGVPDRFSGSGSGTDFTLKISRVEAEDLGVYFCSQTTHVPPTFGGGTKLEIKR

SEQ ID NO: 16 - protein, 113 aa, Artificial Sequence
  Synthetic peptide
DVLMTQTPLSLPVSLGDQASISCRSSQGLVHSNGNTYLHWYLQKPGQSPKLLIYKVSNRFSGVPDRFSGSGSGTDFTLKISRVEAEDLGVYFCSQTTHVPPTFGGGTKLEIKR

SEQ ID NO: 17 - protein, 113 aa, Artificial Sequence
  Synthetic peptide
DVLMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTYLPLTFGAGTRLELKR

SEQ ID NO: 18 - protein, 30 aa, Artificial Sequence
  Synthetic peptide
QVQLQQPGAELVKPGASVKLSCKASGYTFT

SEQ ID NO: 19 - protein, 5 aa, Artificial Sequence
  Synthetic peptide
SYWMQ

SEQ ID NO: 20 - protein, 14 aa, Artificial Sequence
  Synthetic peptide
WVKQRPGQGLEWIG

SEQ ID NO: 21 - protein, 17 aa, Artificial Sequence
  Synthetic peptide
EINPSNGRINYNEKFKS

SEQ ID NO: 22 - protein, 32 aa, Artificial Sequence
  Synthetic peptide
KATLTVDISSSTAYMQLSSLTSEDSAVYYCAR

SEQ ID NO: 23 - protein, 6 aa, Artificial Sequence
  Synthetic peptide
SGLLDY

SEQ ID NO: 24 - protein, 11 aa, Artificial Sequence
  Synthetic peptide
WGQGTTLTDSS

SEQ ID NO: 25 - protein, 30 aa, Artificial Sequence
  Synthetic peptide
EVQLQQSGAELVKPGASVQLSCKASGYTFT

SEQ ID NO: 26 - protein, 17 aa, Artificial Sequence
  Synthetic peptide
EINPSNGRNNYNEKFKR

SEQ ID NO: 27 - protein, 32 aa, Artificial Sequence
  Synthetic peptide
KATLTVDTSSSTAYMQLSSLTSEDSAVYYCAR

SEQ ID NO: 28 - protein, 11 aa, Artificial Sequence
  Synthetic peptide
WGQGTTLTVSS

SEQ ID NO: 29 - protein, 30 aa, Artificial Sequence
  Synthetic peptide
EVQLEESGPSLVKPSQTLSLTCSVTGDSIT

SEQ ID NO: 30 - protein, 5 aa, Artificial Sequence
  Synthetic peptide
SDYWT

SEQ ID NO: 31 - protein, 14 aa, Artificial Sequence
  Synthetic peptide
WIRKFPGNKLEYMG

SEQ ID NO: 32 - protein, 16 aa, Artificial Sequence
  Synthetic peptide
YISYSGRTYYNPSLKS

SEQ ID NO: 33 - protein, 32 aa, Artificial Sequence
  Synthetic peptide
RISITRDTSKKQYYLQLNSVTTEDTATYYCAR

SEQ ID NO: 34 - protein, 10 aa, Artificial Sequence
  Synthetic peptide
ERYYYGSLDY

SEQ ID NO: 35 - protein, 32 aa, Artificial Sequence
  Synthetic peptide
RISITRDTSRNQYYLQLSSVTTEDTATYYCAR

SEQ ID NO: 36 - protein, 14 aa, Artificial Sequence
  Synthetic peptide
WIRKFPGNILEYMG

SEQ ID NO: 37 - protein, 16 aa, Artificial Sequence
  Synthetic peptide
YISSSGRIYYNPSLKS

SEQ ID NO: 38 - protein, 32 aa, Artificial Sequence
  Synthetic peptide
RISITRDTSKNQYYLQLSSVTTEDTATYYCAR

SEQ ID NO: 39 - protein, 5 aa, Artificial Sequence
  Synthetic peptide
SNWMN

SEQ ID NO: 40 - protein, 32 aa, Artificial Sequence
  Synthetic peptide
KATLTVDKSSSTAYMQLSSLTSEDSAVYYCAR

SEQ ID NO: 41 - protein, 23 aa, Artificial Sequence
  Synthetic peptide
DVLMTQTPLSLPVSLGDQASISC

SEQ ID NO: 42 - protein, 16 aa, Artificial Sequence
  Synthetic peptide
RSSQSLIHSNGNTYLH

SEQ ID NO: 43 - protein, 15 aa, Artificial Sequence
  Synthetic peptide
WSLQKPGQSPKLLIY

SEQ ID NO: 44 - protein, 7 aa, Artificial Sequence
  Synthetic peptide
KVSNRFS

SEQ ID NO: 45 - protein, 32 aa, Artificial Sequence
  Synthetic peptide
GVPDRFSGSGSGTDFTLKISSVEAEDLGVYFC

SEQ ID NO: 46 - protein, 9 aa, Artificial Sequence
  Synthetic peptide
SQSTYVPLT

SEQ ID NO: 47 - protein, 11 aa, Artificial Sequence
  Synthetic peptide
FGAGTKLELKR

SEQ ID NO: 48 - protein, 16 aa, Artificial Sequence
  Synthetic peptide
RSSQSLVHSNGNTYLH

SEQ ID NO: 49 - protein, 15 aa, Artificial Sequence
  Synthetic peptide
WYLQKPGQSPKLLIY

SEQ ID NO: 50 - protein, 32 aa, Artificial Sequence
  Synthetic peptide
GVPDRFSGSGSGTDFTLTISRVEAEDLGVYFC

SEQ ID NO: 51 - protein, 23 aa, Artificial Sequence
  Synthetic peptide
DVVMTQTPLSLPVSLGDQASISC

SEQ ID NO: 52 - protein, 32 aa, Artificial Sequence
  Synthetic peptide
GVPDRFSGSGSGTDFTLKISRVEAEDLGVYFC

SEQ ID NO: 53 - protein, 9 aa, Artificial Sequence
  Synthetic peptide
SQTTHVPPT

SEQ ID NO: 54 - protein, 11 aa, Artificial Sequence
  Synthetic peptide
FGGGTKLEIKR

SEQ ID NO: 55 - protein, 16 aa, Artificial Sequence
  Synthetic peptide
RSSQGLVHSNGNTYLH

SEQ ID NO: 56 - protein, 9 aa, Artificial Sequence
  Synthetic peptide
SQSTYLPLT

SEQ ID NO: 57 - protein, 11 aa, Artificial Sequence
  Synthetic peptide
FGAGTRLELKR

SEQ ID NO: 58 - protein, 5 aa, Artificial Sequence
  Synthetic peptide
  MISC_FEATURE (2)..(2): Xaa is Tyr or Asn
  MISC_FEATURE (5)..(5): Xaa is Gln or Asn
SXWMX

SEQ ID NO: 59 - protein, 17 aa, Artificial Sequence
  Synthetic peptide
  MISC_FEATURE (9)..(9): Xaa is Ile or Asn
  MISC_FEATURE (17)..(17): Xaa is Ser or Arg
EINPSNGRXNYNEKFKX

SEQ ID NO: 60 - protein, 16 aa, Artificial Sequence
  Synthetic peptide
  MISC_FEATURE (7)..(7): Xaa is Ile or Val
RSSQSLXHSNGNTYLH

SEQ ID NO: 61 - protein, 9 aa, Artificial Sequence
  Synthetic peptide
  MISC_FEATURE (6)..(6): Xaa is Val or Leu
SQSTYXPLT

SEQ ID NO: 62 - protein, 16 aa, Artificial Sequence
  Synthetic peptide
  MISC_FEATURE (4)..(4): Xaa is Tyr or Ser
  MISC_FEATURE (8)..(8): Xaa is Thr or Ile
YISXSGRXYYNPSLKS

SEQ ID NO: 63 - protein, 16 aa, Artificial Sequence
  Synthetic peptide
  MISC_FEATURE (5)..(5): Xaa is Ser or Gly
RSSQXLVHSNGNTYLH

SEQ ID NO: 64 - DNA, 345 bp, Artificial Sequence
  Synthetic polynucleotide
caggtccaactgcagcagcctggggctgaactggtgaagcctggggcttcagtgaagctgtcctgcaaggcttctggctacaccttcaccagctactggatgcagtgggtgaagcagaggcctggacaaggccttgagtggattggagagattaatcctagcaacggtcgtattaactacaatgagaagttcaagagcaaggccacacttactgtagacatatcgtccagcacagcctacatgcaactcagcagtctgacatctgaggactctgcggtctattactgtgcaagatcggggctccttgactactggggccaaggcaccactctcacagactcctca

SEQ ID NO: 65 - DNA, 345 bp, Artificial Sequence
  Synthetic polynucleotide
gaggtccagctgcaacagtctggggctgaactggtgaagcctggggcttcagtgcagctgtcctgcaaggcttctggctacaccttcaccagctactggatgcagtgggtgaaacagaggcctggacaaggccttgagtggattggagagattaatcctagcaacggtcgcaataattacaatgagaagttcaagagaaaggccacacttactgttgacacatcctccagcacagcctacatgcaactcagcagcctgacatctgaggactctgcggtctattactgtgcaagatcggggctccttgactactggggccaaggcaccactctcacagtctcctca

SEQ ID NO: 66 - DNA, 354 bp, Artificial Sequence
  Synthetic polynucleotide
gaggtgcagctggaggagtcaggacctagcctcgtgaaaccttctcagactctgtccctcacctgttctgtcactggcgactccatcaccagtgattactggacctggatccggaaattcccagggaataaacttgagtacatggggtacataagctacagtggtagaacttactacaatccatctctcaaaagtcgaatctccatcactcgagacacatccaagaagcagtactacctgcagttgaattctgtgacaactgaggacacagccacatattactgtgcaagagagcgttattactacggtagtcttgactactggggccaaggcaccactctcacagtctcctca

SEQ ID NO: 67 - DNA, 354 bp, Artificial Sequence
  Synthetic polynucleotide
gaggtgcagctggaggagtcaggacctagcctcgtgaaaccttctcagactctgtccctcacctgttctgtcactggcgactccatcaccagtgattactggacctggatccggaaattcccagggaataaacttgagtacatggggtacataagctacagtggtagaacttactacaatccatctctcaaaagtcgaatctccatcactcgagacacatccaggaaccagtactacctgcagttgagttctgtgactactgaggacacagccacatattactgtgcaagagagcgttattactacggtagtcttgactactggggccagggcaccactctcacagtctcctca

SEQ ID NO: 68 - DNA, 354 bp, Artificial Sequence
  Synethetic polynucleotide
gaggtgcagctggaggagtcaggacctagcctcgtgaaaccttctcagactctgtccctcacctgttctgtcactggcgactccatcaccagtgattactggacctggatccggaaattcccagggaatatacttgagtacatggggtacataagctccagtggtaggatttattacaatccatctctcaaaagtcgaatctccatcactcgagacacatccaagaaccagtactacctgcagttgagttctgtgactactgaggacacagccacatattactgtgcaagagagcgttattactacggtagtcttgactactggggccaaggcaccactctcacagtctcctca

SEQ ID NO: 69 - DNA, 345 bp, Artificial Sequence
  Synthetic polynucleotide
caggtccaactgcagcagcctggggctgaactggtgaagcctggggcttcagtgaagctgtcctgcaaggcttctggctacaccttcaccagcaactggatgaactgggtgaagcagaggcctggacaaggccttgagtggattggagagattaatcctagcaacggtcgtattaattacaatgagaagttcaagagcaaggccacacttactgtggacaaatcctccagcacagcctacatgcaactcagcagcctgacatctgaggactctgcggtctattactgtgcaagatcggggctccttgactactggggccaaggcaccactctcacagtctcctca

SEQ ID NO: 70 - DNA, 339 bp, Artificial Sequence
  Synthetic polynucleotide
gatgttttgatgacccaaactccactctccctgcctgtcagtcttggagatcaagcctccatctcttgcagatctagtcagagccttatacacagtaatggaaacacctatttacattggtccctgcagaagccaggccagtctccaaagctcctgatctacaaagtttccaaccgattttctggggtcccagacaggttcagtggcagtggatcagggacagatttcacactcaagatcagcagcgtggaggctgaggatctgggagtttatttctgctctcaaagtacatatgtcccgctcacgttcggtgctgggaccaagctggagctgaaacgg

SEQ ID NO: 71 - DNA, 339 bp, Artificial Sequence
  Synthetic polynucleotide
gatgttttgatgacccaaactccactctccctgcctgtcagtcttggagatcaagcctccatctcttgcagatctagtcagagccttgtacacagtaatggcaacacctatttacattggtacctgcagaagccaggccagtctccaaagctcctgatctacaaagtttctaaccgattttctggggtcccagacaggttcagtggcagtggatcagggacagatttcacactcacgatcagcagagtggaggctgaggatctgggagtttatttctgctctcaaagtacatatgtcccgctcacgttcggtgctgggaccaagctggagctgaaacgg

SEQ ID NO: 72 - DNA, 339 bp, Artificial Sequence
  Synthetic polynucleotide
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatttacaaagtttccaaccgattttctggggtcccagacaggttcagtggcagtggatcagggacagatttcacactcaagatcagcagagtggaggctgaggatctgggagtttatttctgctctcaaactacacatgttcctcccacgttcggaggggggaccaagctggaaataaaacgg

SEQ ID NO: 73 - DNA, 339 bp, Artificial Sequence
  Synthetic polynucleotide
gatgttttgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaactacacatgttcctcccacgttcggaggggggaccaagctggaaataaaacgg

SEQ ID NO: 74 - DNA, 339 bp, Artificial Sequence
  Synthetic polynucleotide
gatgttttgatgacccaaactccactctccctgcctgtcagtcttggagatcaagcctccatctcttgcagatctagtcagggccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaactacacatgttcctcccacgttcggaggggggaccaagctggaaataaaacgg

SEQ ID NO: 75 - DNA, 339 bp, Artificial Sequence
  Synthetic polynucleotide
gatgttttgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacatatcttccgctcacgttcggtgctgggaccaggctggagctgaaacgg

SEQ ID NO: 76 - protein, 122 aa, Artificial Sequence
  Synthetic peptide
EVQLEESGAELVRSGASVKLSCTASGFNIKDYYLHWVKQRPEQGLEWIGWIDPENGNTDYAPKFQGKATMTADTSSNTAYLQLSSLTSEDTAVYYCNEGYYDYDTDSAMDYWGQGTSVTVSS

SEQ ID NO: 77 - protein, 113 aa, Artificial Sequence
  Synthetic peptide
DVLMTQTPLSLPVSLGDQASISCRSSQSLVHSDGITYLHWYLQKPGQSPKLLIYKVSNRFSGVPDRFSGSGSGTDFTLKISRVEAEDLGVYFCSQSTRVPWTFGGGTKLEIKR

SEQ ID NO: 78 - protein, 30 aa, Artificial Sequence
  Synthetic peptide
EVQLEESGAELVRSGASVKLSCTASGFNIK

SEQ ID NO: 79 - protein, 5 aa, Artificial Sequence
  Synthetic peptide
DYYLH

SEQ ID NO: 80 - protein, 14 aa, Artificial Sequence
  Synthetic peptide
WVKQRPEQGLEWIG

SEQ ID NO: 81 - protein, 17 aa, Artificial Sequence
  Synthetic peptide
WIDPENGNTDYAPKFQG

SEQ ID NO: 82 - protein, 32 aa, Artificial Sequence
  Synthetic peptide
KATMTADTSSNTAYLQLSSLTSEDTAVYYCNE

SEQ ID NO: 83 - protein, 13 aa, Artificial Sequence
  Synthetic peptide
GYYDYDTDSAMDY

SEQ ID NO: 84 - protein, 11 aa, Artificial Sequence
  Synthetic peptide
WGQGTSVTVSS

SEQ ID NO: 85 - protein, 23 aa, Artificial Sequence
  Synthetic peptide
DVLMTQTPLSLPVSLGDQASISC

SEQ ID NO: 86 - protein, 16 aa, Artificial Sequence
  Synthetic peptide
RSSQSLVHSDGITYLH

SEQ ID NO: 87 - protein, 15 aa, Artificial Sequence
  Synthetic peptide
WYLQKPGQSPKLLIY

SEQ ID NO: 88 - protein, 7 aa, Artificial Sequence
  Synthetic peptide
KVSNRFS

SEQ ID NO: 89 - protein, 32 aa, Artificial Sequence; identical to SEQ ID NO: 52
  Synthetic peptide

SEQ ID NO: 90 - protein, 9 aa, Artificial Sequence
  Synthetic peptide
SQSTRVPWT

SEQ ID NO: 91 - protein, 11 aa, Artificial Sequence
  Synthetic peptide
FGGGTKLEIKR

SEQ ID NO: 92 - DNA, 366 bp, Artificial Sequence
  Synthetic polynucleotide
gaggtgcagctggaggagtctggggcagagcttgtgaggtcaggggcctcagtcaagttgtcctgcacagcttctggcttcaacattaaagactactatttgcactgggtgaagcagaggcctgaacagggcctggagtggattggatggattgatcctgagaatggtaatactgattatgccccgaagttccagggcaaggccactatgactgcagacacatcctccaacacagcctacctgcagctcagcagcctgacatctgaggacactgccgtctattactgtaatgaggggtattatgattacgacacagactctgctatggactactggggtcaaggaacctcagtcaccgtctcctca

SEQ ID NO: 93 - DNA, 339 bp, Artificial Sequence
  Synthetic polynucleotide
gatgttttgatgacccaaactccactctccctgcctgtcagtcttggagatcaagcctccatctcttgcagatctagtcagagccttgtacacagtgatggaatcacctatttacattggtacctacagaagccaggccagtctccaaaactcctgatctacaaagtttccaaccgattttctggggtcccagacaggttcagtggcagtggatcagggacagatttcacactcaagatcagcagagtggaggctgaggatctgggagtttatttctgctctcaaagtacacgtgttccgtggacgttcggtggaggcaccaagctggaaatcaaacgg

SEQ ID NO: 94 - protein, 119 aa, Artificial Sequence
  Synthetic peptide
EVQLQQSGTVLARPGASVKMSCKASGYTFTNYWMHWVKQRPGQGLEWIGAIYPGNSDTSYNQKFKGKAKLTAVTSASTAYMELSSLTNEDSAVYFCTRADYDGTPFDYWGQGTTLTVSS

SEQ ID NO: 95 - protein, 119 aa, Artificial Sequence
  Synthetic peptide
EVQLQQSGTVLARPGASVRMSCRASGYSFNSYWMHWVKQRPGQGLEWIGAIYPGSSDTSYSQKFKGKAKLTAVTSANTAYMELSSLTNEDSAVYYCTRGDYDGTPFDYWGQGTTLTVSS

SEQ ID NO: 96 - protein, 119 aa, Artificial Sequence
  Synthetic peptide
EVQLQQSGTVLARPGTSVKMSCKASGYTFTSYWMHWVKQRPGQGLEWIGAIYLGNTDTSYNQKFKGKAKLTAVTSASSAYMELSSLTNEDSAVYYCTRADYDGTPFDYWGQGTTLTVSS

SEQ ID NO: 97 - protein, 119 aa, Artificial Sequence
  Synthetic peptide
EVQLQQSGTVLARPGASVKMSCRPSGYTFTSYWMHWVKQRPGQDLEWIGAIYPGNSDTSYNQKFKGKAKLTAVTSASTAYMELSSLTNEDSAVYYCTRADYDGTPFDYWGQGTTLTVSS

SEQ ID NO: 98 - protein, 119 aa, Artificial Sequence; identical to SEQ ID NO: 95
  Synthetic peptide

SEQ ID NO: 99 - protein, 122 aa, Artificial Sequence
  Synthetic peptide
EVQLEESGAELVRSGASVKLSCTASGFNIKDYYLHWVKQRPEQGLEWIGWIDPENGATDYAPKFQGKASMTADTSSNTAYLQLSSLTFEDTAVYYCNEGYYDYDADSAMDYWGQGTSVTVSS

SEQ ID NO: 100 - protein, 119 aa, Artificial Sequence
  Synthetic peptide
EVQLQQSGTVLARPGASVKMSCQASGYTFTNYWMHWVKQRPGQGLEWIGAIYPGNSDTSYNQNFKGKAELTAVTSATTAYMELSSLTDEDSAVYYCTRADYDGTPFDYWGQGTTLTVSS

SEQ ID NO: 101 - protein, 119 aa, Artificial Sequence
  Synthetic peptide
EVQLQQSGTVLARPGASVKMSCKASGYTFTNYWMHWVKQRPGQGLEWIGAVYPGNSDTSYSQKFTGKAKLTAVTSASTAYMDLSSLTNEDSAVYYCTRADYDGTPFDYWGQGTTLTVSS

SEQ ID NO: 102 - protein, 119 aa, Artificial Sequence
  Synthetic peptide
EVQLQQSGTVLARPGASVKMSCKASGYTFTSYWMHWVKQRPGQGLEWIGAIYPGNSDTSYNQKFKGKARLTAVTSASTAYMELSSLTNEDSAVYYCTRADYDGTPFDYWGQGTTLTVSS

SEQ ID NO: 103 - protein, 119 aa, Artificial Sequence
  Synthetic peptide
EVQLQQSGTVLARPGASVKMSCKASGYSFTSYWMHWVKQRPGQGLEWIGAIYPGNSDTSYNQKFKGKAKLTAVTSASTAYMELSSLTNEDSAVYYCTRGDYDGTPFDYWGQGTTLTVSS

SEQ ID NO: 104 - protein, 113 aa, Artificial Sequence
  Synthetic peptide
DILMTQSPLTLSVTIGHPASISCKSSQSLLDSDGETYLSWLLQRPGQSPERLIYLVSKLDSGVPDRFTGSGSGTDFTLKISRVEAEDLGVYYCWQGTHFPLTFGAGTKLELKR

SEQ ID NO: 105 - protein, 113 aa, Artificial Sequence
  Synthetic peptide
DILMTQSPLTLSVTIGQPASISCKSGQSLLDSDGKTYLNWLLQRPGQSPKRLIYLVSKLHSGVPDRFTGSGSGTDFTLKISRVEAEDLGVYYCWQGTHFPLTFGAGTKLELKR

SEQ ID NO: 106 - protein, 113 aa, Artificial Sequence
  Synthetic peptide
DVVMTQNALTLSVTIGHPASISCRSSQSLLDSDGETYLSWLLQRPGQSPKRLIYLVSKLDSGVPDRFTGSGSGTDFTLKISRVEAEDLGVYYCWQGTHFPLTFGAGTKLELKR

SEQ ID NO: 107 - protein, 113 aa, Artificial Sequence
  Synthetic peptide
DIVMTQSPLTLSVTIGHPASISCKSSQSLLDSDGETYLSWLLQRPGQSPKRLIYLVSKLDSGVPDRFTGSGSGTDFTLKISRVEAEDLGVYYCWQGTHFPLTFGAGTKLELKR

SEQ ID NO: 108 - protein, 113 aa, Artificial Sequence
  Synthetic peptide
DIVMTQSPLTLSVTIGQPASISCKSGQSLLDSDGKTYLNWLLQRPGQSPKRLIYLVSKLHSGVPDRFTGSGSGTDFTLKISRVEAEDLGVYYCWQGTHFPLTFGAGTKLELKR

SEQ ID NO: 109 - protein, 113 aa, Artificial Sequence
  Synthetic peptide
DVLMTQTPLSLPVSLGDQASISCRSSQSLVHSDGITYLHWYLQKPGQSPKLLIYKVSNRFSGVPDRFSGSGSGTDFTLKISRVEAEDLGVYFCSQSARVPWTFGGGTKLEIKR

SEQ ID NO: 110 - protein, 113 aa, Artificial Sequence
  Synthetic peptide
DIVMTQSPLTLSVTIGHPASISCKSSQSLLDSDGETYLSWLLQRPGQSPKRLIYLVSKLDSGVPDRFTGSGSGTDFTLKISRVEAEDLGVYYCWQGTHFPLTFGAGTKVELKR

SEQ ID NO: 111 - protein, 113 aa, Artificial Sequence
  Synthetic peptide
DVLMTQTPLTLSVIIGQPASISCKSSQSLLDSDGETYLSWLLQRPGQSPKRLIYLVSKLDSGVPDRFTGSGSGTDFTLKISRVEAEDLGVYYCWQGTHFPLTFGAGTKLELKR

SEQ ID NO: 112 - protein, 113 aa, Artificial Sequence; identical to SEQ ID NO: 107
  Synthetic peptide

SEQ ID NO: 113 - protein, 113 aa, Artificial Sequence
  Synthetic peptide
DVLMTQTPLTLSVTIGQPASISCKSSQSLLDSDGETYLNWLLQRPGQSPKRLIYLASKLDSGVPDRFTGSGSGTDFTLKISRVEAEDLGIYYCWQGTHFPLTFGAGTKLELKR

SEQ ID NO: 114 - protein, 30 aa, Artificial Sequence
  Synthetic peptide
EVQLQQSGTVLARPGASVKMSCKASGYTFT

SEQ ID NO: 115 - protein, 5 aa, Artificial Sequence
  Synthetic peptide
NYWMH

SEQ ID NO: 116 - protein, 30 aa, Artificial Sequence
  Synthetic peptide
EVQLQQSGTVLARPGASVRMSCRASGYSFN

SEQ ID NO: 117 - protein, 5 aa, Artificial Sequence
  Synthetic peptide
SYWMH

SEQ ID NO: 118 - protein, 30 aa, Artificial Sequence
  Synthetic peptide
EVQLQQSGTVLARPGTSVKMSCKASGYTFT

SEQ ID NO: 119 - protein, 30 aa, Artificial Sequence
  Synthetic peptide
EVQLQQSGTVLARPGASVKMSCRPSGYTFT

SEQ ID NO: 120 - protein, 30 aa, Artificial Sequence
  Synthetic peptide
EVQLQQSGTVLARPGASVKMSCQASGYTFT

SEQ ID NO: 121 - protein, 30 aa, Artificial Sequence
  Synthetic peptide
EVQLQQSGTVLARPGASVKMSCKASGYSFT

SEQ ID NO: 122 - protein, 14 aa, Artificial Sequence
  Synthetic peptide
WVKQRPGQGLEWIG

SEQ ID NO: 123 - protein, 17 aa, Artificial Sequence
  Synthetic peptide
AIYPGNSDTSYNQKFKG

SEQ ID NO: 124 - protein, 17 aa, Artificial Sequence
  Synthetic peptide
AIYPGSSDTSYSQKFKG

SEQ ID NO: 125 - protein, 17 aa, Artificial Sequence
  Synthetic peptide
AIYLGNTDTSYNQKFKG

SEQ ID NO: 126 - protein, 14 aa, Artificial Sequence
  Synthetic peptide
WVKQRPGQDLEWIG

SEQ ID NO: 127 - protein, 17 aa, Artificial Sequence
  Synthetic peptide
WIDPENGATDYAPKFQG

SEQ ID NO: 128 - protein, 17 aa, Artificial Sequence
  Synthetic peptide
AIYPGNSDTSYNQNFKG

SEQ ID NO: 129 - protein, 17 aa, Artificial Sequence
  Synthetic peptide
AVYPGNSDTSYSQKFTG

SEQ ID NO: 130 - protein, 32 aa, Artificial Sequence
  Synthetic peptide
KAKLTAVTSASTAYMELSSLTNEDSAVYFCTR

SEQ ID NO: 131 - protein, 10 aa, Artificial Sequence
  Synthetic peptide
ADYDGTPFDY

SEQ ID NO: 132 - protein, 32 aa, Artificial Sequence
  Synthetic peptide
KAKLTAVTSANTAYMELSSLTNEDSAVYYCTR

SEQ ID NO: 133 - protein, 10 aa, Artificial Sequence
  Synthetic peptide
GDYDGTPFDY

SEQ ID NO: 134 - protein, 32 aa, Artificial Sequence
  Synthetic peptide
KAKLTAVTSASSAYMELSSLTNEDSAVYYCTR

SEQ ID NO: 135 - protein, 32 aa, Artificial Sequence
  Synthetic peptide
KAKLTAVTSASTAYMELSSLTNEDSAVYYCTR

SEQ ID NO: 136 - protein, 32 aa, Artificial Sequence
  Synthetic peptide
KASMTADTSSNTAYLQLSSLTFEDTAVYYCNE

SEQ ID NO: 137 - protein, 13 aa, Artificial Sequence
  Synthetic peptide
GYYDYDADSAMDY

SEQ ID NO: 138 - protein, 32 aa, Artificial Sequence
  Synthetic peptide
KAELTAVTSATTAYMELSSLTDEDSAVYYCTR

SEQ ID NO: 139 - protein, 32 aa, Artificial Sequence
  Synthetic peptide
KAKLTAVTSASTAYMDLSSLTNEDSAVYYCTR

SEQ ID NO: 140 - protein, 32 aa, Artificial Sequence
  Synthetic peptide
KARLTAVTSASTAYMELSSLTNEDSAVYYCTR

SEQ ID NO: 141 - protein, 11 aa, Artificial Sequence
  Synthetic peptide
WGQGTTLTVSS

SEQ ID NO: 142 - protein, 23 aa, Artificial Sequence
  Synthetic peptide
DILMTQSPLTLSVTIGHPASISC

SEQ ID NO: 143 - protein, 16 aa, Artificial Sequence
  Synthetic peptide
KSSQSLLDSDGETYLS

SEQ ID NO: 144 - protein, 23 aa, Artificial Sequence
  Synthetic peptide
DILMTQSPLTLSVTIGQPASISC

SEQ ID NO: 145 - protein, 16 aa, Artificial Sequence
  Synthetic peptide
KSGQSLLDSDGKTYLN

SEQ ID NO: 146 - protein, 23 aa, Artificial Sequence
  Synthetic peptide
DVVMTQNALTLSVTIGHPASISC

SEQ ID NO: 147 - protein, 16 aa, Artificial Sequence
  Synthetic peptide
RSSQSLLDSDGETYLS

SEQ ID NO: 148 - protein, 23 aa, Artificial Sequence
  Synthetic peptide
DIVMTQSPLTLSVTIGHPASISC

SEQ ID NO: 149 - protein, 23 aa, Artificial Sequence
  Synthetic peptide
DIVMTQSPLTLSVTIGQPASISC

SEQ ID NO: 150 - protein, 23 aa, Artificial Sequence
  Synthetic peptide
DVLMTQTPLTLSVIIGQPASISC

SEQ ID NO: 151 - protein, 23 aa, Artificial Sequence
  Synthetic peptide
DVLMTQTPLTLSVTIGQPASISC

SEQ ID NO: 152 - protein, 16 aa, Artificial Sequence
  Synthetic peptide
KSSQSLLDSDGETYLN

SEQ ID NO: 153 - protein, 15 aa, Artificial Sequence
  Synthetic peptide
WLLQRPGQSPERLIY

SEQ ID NO: 154 - protein, 7 aa, Artificial Sequence
  Synthetic peptide
LVSKLDS

SEQ ID NO: 155 - protein, 15 aa, Artificial Sequence
  Synthetic peptide
WLLQRPGQSPKRLIY

SEQ ID NO: 156 - protein, 7 aa, Artificial Sequence
  Synthetic peptide
LVSKLHS

SEQ ID NO: 157 - protein, 7 aa, Artificial Sequence
  Synthetic peptide
LASKLDS

SEQ ID NO: 158 - protein, 32 aa, Artificial Sequence
  Synthetic peptide
GVPDRFTGSGSGTDFTLKISRVEAEDLGVYYC

SEQ ID NO: 159 - protein, 9 aa, Artificial Sequence
  Synthetic peptide
WQGTHFPLT

SEQ ID NO: 160 - protein, 9 aa, Artificial Sequence
  Synthetic peptide
SQSARVPWT

SEQ ID NO: 161 - protein, 32 aa, Artificial Sequence
  Synthetic peptide
GVPDRFTGSGSGTDFTLKISRVEAEDLGIYYC

SEQ ID NO: 162 - protein, 11 aa, Artificial Sequence
  Synthetic peptide
FGAGTKLELKR

SEQ ID NO: 163 - protein, 11 aa, Artificial Sequence
  Synthetic peptide
FGAGTKVELKR

SEQ ID NO: 164 - protein, 5 aa, Artificial Sequence
  Synthetic peptide
  MISC_FEATURE (1)..(1): Xaa is Asn or Ser
XYWMH

SEQ ID NO: 165 - protein, 17 aa, Artificial Sequence
  Synthetic peptide
  MISC_FEATURE (2)..(2): Xaa is Ile or Val
  MISC_FEATURE (4)..(4): Xaa is Pro or Leu
  MISC_FEATURE (6)..(6): Xaa is Asn or Ser
  MISC_FEATURE (7)..(7): Xaa is Ser or Thr
  MISC_FEATURE (12)..(12): Xaa is Asn or Ser
  MISC_FEATURE (14)..(14): Xaa is Lys or Asn
  MISC_FEATURE (16)..(16): Xaa is Lys or Thr
AXYXGXXDTSYXQXFXG

SEQ ID NO: 166 - protein, 10 aa, Artificial Sequence
  Synthetic peptide
  MISC_FEATURE (1)..(1): Xaa is Arg or Gly
XDYDGTPFDY

SEQ ID NO: 167 - protein, 16 aa, Artificial Sequence
  Synthetic peptide
  MISC_FEATURE (1)..(1): Xaa is Lys or Arg
  MISC_FEATURE (3)..(3): Xaa is Ser or Gly
  MISC_FEATURE (12)..(12): Xaa is Glu or Lys
  MISC_FEATURE (16)..(16): Xaa is Ser or Asn
XSXQSLLDSDGXTYLX

SEQ ID NO: 168 - protein, 7 aa, Artificial Sequence
  Synthetic peptide
  MISC_FEATURE (2)..(2): Xaa is Val or Ala
  MISC_FEATURE (6)..(6): Xaa is Asp or His
LXSKLXS

SEQ ID NO: 169 - protein, 17 aa, Artificial Sequence
  Synthetic peptide
  MISC_FEATURE (8)..(8): Xaa is Ala or Asn
WIDPENGXTDYAPKFQG

SEQ ID NO: 170 - protein, 13 aa, Artificial Sequence
  Synthetic peptide
  MISC_FEATURE (7)..(7): Xaa is Ala or Thr
GYYDYDXDSAMDY

SEQ ID NO: 171 - protein, 9 aa, Artificial Sequence
  Synthetic peptide
  MISC_FEATURE (4)..(4): Xaa is Ala or Thr
SQTXHVPPT

SEQ ID NO: 172 - DNA, 357 bp, Artificial Sequence
  Synthetic polynucleotide
gaggttcaactccagcagtctgggactgtgctggcaaggcctggggcttccgtgaagatgtcctgcaaggcttctggctacacctttaccaactactggatgcactgggtaaaacagaggcctggacagggtctagaatggattggtgctatttatcctggaaatagtgatactagctacaaccagaagttcaagggcaaggccaaactgactgcagtcacatccgccagcactgcctacatggagctcagcagcctgacaaatgaggactctgcggtctatttctgtacaagggctgattacgacgggaccccctttgactactggggccaaggcaccactctcacagtctcctca

SEQ ID NO: 173 - DNA, 357 bp, Artificial Sequence
  Synthetic polynucleotide
gaggttcagctccagcagtctgggactgtgctggcaaggcctggggcttccgtgaggatgtcctgcagggcttctggctacagctttaacagctactggatgcactgggtaaaacagaggcctggacagggtctagaatggattggtgctatttatcctggaagtagtgatactagctacagccagaagttcaagggcaaggccaaactgactgcagtcacatccgccaacactgcctacatggagctcagcagcctgacaaatgaggactctgcggtctattactgtacaaggggtgattacgacgggaccccctttgactactggggccaaggcaccactctcacagtctcctca

SEQ ID NO: 174 - DNA, 357 bp, Artificial Sequence
  Synthetic polynucleotide
gaggttcaactccagcagtctgggactgtgctggcaaggcctgggacttccgtgaagatgtcctgcaaggcttctggctacacctttaccagctactggatgcactgggtaaaacagaggcctggacagggtctagaatggattggtgccatttatcttggaaatactgatactagctacaaccagaagttcaagggcaaggccaaactgactgcagtcacatccgccagcagtgcctacatggagctcagcagcctgacaaatgaggactctgcggtctattattgtacaagggctgattacgacgggaccccctttgactactggggccaaggcaccactctcacagtctcctca

SEQ ID NO: 175 - DNA, 357 bp, Artificial Sequence
  Synthetic polynucleotide
gaggttcaactccagcagtctgggactgtgctggcaaggcctggggcttccgtgaagatgtcctgcaggccttctggctacacctttaccagctactggatgcactgggtaaaacagaggcctggacaggatctagaatggattggtgctatttatcctggaaatagtgatactagctacaaccagaagttcaagggcaaggccaaactgactgcagtcacatccgccagcactgcctacatggagctcagcagcctgacaaatgaggactctgcggtctattactgtacaagggctgattacgacgggaccccctttgactactggggccaaggcaccactctcacagtctcctca

SEQ ID NO: 176 - DNA, 357 bp, Artificial Sequence; identical to SEQ ID NO: 173
  Synthetic polynucleotide

SEQ ID NO: 177 - DNA, 366 bp, Artificial Sequence
  Synthetic polynucleotide
gaggtgcagctggaggagtctggggcagagcttgtgaggtcaggggcctcagtcaagttgtcctgcacagcttctggcttcaacattaaagactactatttacactgggtgaagcagaggcctgaacagggcctggagtggattggatggattgatcctgagaatggtgccactgattatgccccgaagttccagggcaaggcctctatgactgcagacacatcctccaacacagcctacctgcagctcagcagcctgacatttgaggacactgccgtctattattgtaatgaggggtattatgattacgacgcggactctgctatggactactggggtcaaggaacctcagtcaccgtctcctca

SEQ ID NO: 178 - DNA, 357 bp, Artificial Sequence
  Synthetic polynucleotide
gaggttcaactccagcagtctgggactgtgctggcaaggcctggggcttccgtgaagatgtcctgccaggcttctggctacacctttaccaactactggatgcactgggtaaaacagaggcctggacagggtctagaatggattggtgctatctatcctggaaatagtgatactagctataaccagaatttcaagggcaaggccgaactgactgcagtcacatccgccaccactgcctacatggaactcagcagcctgacagatgaagactctgcggtctattactgtacaagggctgattacgacgggaccccctttgactactggggccaaggcaccactctcacagtctcctca

SEQ ID NO: 179 - DNA, 357 bp, Artificial Sequence
  Synthetic polynucleotide
gaggttcaactccagcagtctgggactgtgctggcaaggcctggggcttccgtgaagatgtcctgcaaggcttctggctacacctttaccaactactggatgcactgggtaaaacagaggcctggacagggtctagaatggattggtgctgtttatcctggaaacagtgatactagttacagccagaagttcacgggcaaggccaaactgactgcagtcacatccgccagcactgcctacatggacctcagcagcctgacaaatgaggactctgcggtctattactgtacaagggctgattacgacgggaccccctttgactactggggccaaggcactactctcacagtctcctca

SEQ ID NO: 180 - DNA, 357 bp, Artificial Sequence
  Synthetic polynucleotide
gaggttcaactccagcagtctgggactgtgctggcaaggcctggggcttccgtgaagatgtcctgcaaggcttctggctacacctttaccagctactggatgcactgggtaaaacagaggcctggacagggtctagaatggattggtgctatttatcctggaaatagtgatactagctacaaccagaagttcaagggcaaggccagactgactgcagtcacatccgccagcactgcctacatggagctcagcagcctgacaaatgaggactctgcggtctattattgtacaagggctgattacgacgggaccccctttgactactggggccaaggcaccactctcacagtctcctca

SEQ ID NO: 181 - DNA, 357 bp, Artificial Sequence
  Synthetic polynucleotide
gaggttcagctccagcagtctgggactgtgctggcaaggcctggggcttccgtgaagatgtcctgcaaggcttctggctacagctttaccagctactggatgcactgggtaaaacagaggcctggacagggtctagaatggattggtgctatttatcctggaaatagtgatactagctacaaccagaagttcaagggcaaggccaaactgactgcagtcacatccgccagcactgcctacatggagctcagcagcctgacaaatgaggactctgcggtctattactgtacaaggggtgattacgacgggaccccctttgactactggggccaaggcaccactctcacagtctcctca

SEQ ID NO: 182 - DNA, 339 bp, Artificial Sequence
  Synthetic polynucleotide
gacattctgatgacccagtctccactcactttgtcggttaccattggacacccagcctccatctcttgcaagtcaagtcagagcctcttagatagtgatggagagacatatttgagttggttgttacagaggccaggccagtctccagagcgcctaatctatctggtgtctaaactggactctggagtccctgacaggttcactggcagtggatcagggacagatttcacactgaaaatcagcagagtggaggctgaggatttgggagtttattattgttggcaaggtacacattttccgctcacgttcggtgctgggaccaagctggagctgaaacgg

SEQ ID NO: 183 - DNA, 339 bp, Artificial Sequence
  Synthetic polynucleotide
gacattctgatgacccagtctccactcactttgtcggttaccattggacaaccagcctccatctcttgcaagtcaggtcagagcctcttagatagtgatggaaagacatatttgaattggttgttacagaggccaggccagtctccaaagcgcctaatctatctggtgtctaaactgcactctggagtccctgacaggttcactggcagtggatcagggacagatttcacactgaaaatcagcagagtggaggctgaggatttgggagtttattattgctggcaaggtacacattttccgctcacgttcggtgctgggaccaagctggagctgaaacgg

SEQ ID NO: 184 - DNA, 339 bp, Artificial Sequence
  Synthetic polynucleotide
gatgttgtgatgacccagaatgcactcactttgtcggttaccatcggacacccagcctccatctcttgtaggtcaagtcagagcctcttagatagtgatggagagacatatttgagttggttgttacagaggccaggccagtctccaaagcgcctaatctatctggtgtctaaattggactctggagtccctgacaggttcactggcagtggatcagggacagatttcacactgaaaatcagcagagtggaggctgaggatttgggagtttattattgttggcaaggcacacattttccgctcacgttcggtgctgggaccaagctggagctgaaacgg

SEQ ID NO: 185 - DNA, 339 bp, Artificial Sequence
  Synthetic polynucleotide
gacattgtgatgacccagtctccactcactttgtcggttaccattggacacccagcctccatctcttgcaagtcaagtcagagcctcttagatagtgatggagagacatatttgagttggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgttggcaaggtacacattttccgctcacgttcggtgctgggaccaagctggagctgaaacgg

SEQ ID NO: 186 - DNA, 339 bp, Artificial Sequence
  Synthetic polynucleotide
gacattgtgatgacccagtctccactcactttgtcggttaccattggacaaccagcctccatctcttgcaagtcaggtcagagcctcttagatagtgatggaaagacatatttgaattggttgttacagaggccaggccagtctccaaagcgcctaatctatctggtgtctaaactgcactctggagtccctgacaggttcactggcagtggatcagggacagatttcacactgaaaatcagcagagtggaggctgaggatttgggagtttattattgctggcaaggtacacattttccgctcacgttcggtgctgggaccaagctggagctgaaacgg

SEQ ID NO: 187 - DNA, 339 bp, Artificial Sequence
  Synthetic polynucleotide
gatgttttgatgacccaaactccactctccctgcctgtcagtcttggagatcaagcctccatctcttgcagatctagtcagagccttgtacacagtgatggaatcacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtgcacgtgttccgtggacattcggtggaggcaccaagctggaaatcaaacgg

SEQ ID NO: 188 - DNA, 339 bp, Artificial Sequence
  Synthetic polynucleotide
gacattgtgatgacccagtctccactcactttgtcggttaccattggacacccagcctccatctcttgcaagtcaagtcagagcctcttagatagtgatggagagacatatttgagttggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgttggcaaggtacacattttccgctcacgttcggtgctgggaccaaggtggagctgaaacgg

SEQ ID NO: 189 - DNA, 339 bp, Artificial Sequence
  Synthetic polynucleotide
gatgttttgatgacccaaactccactcactttgtcggttatcattggacagccagcctccatctcttgcaagtcaagtcagagcctcttagatagtgatggagagacatatttgagttggttgttacagaggccaggccagtctccaaagcgcctaatctatctggtgtctaaactggactctggagtccctgaccggttcactggcagtggatcagggacagatttcacactgaaaatcagcagagtggaggctgaggatttgggagtttattattgttggcaaggtacacattttccgctcacgttcggtgctgggaccaagctggagctgaaacgg

SEQ ID NO: 190 - DNA, 339 bp, Artificial Sequence; identical to SEQ ID NO: 185
  Synthetic polynucleotide

SEQ ID NO: 191 - DNA, 339 bp, Artificial Sequence
  Synthetic polynucleotide
gatgttttgatgacccaaactccactcactttgtcggttaccattggacaaccagcctccatctcttgcaagtcaagtcagagcctcttagatagtgatggagagacatatttgaattggttgttacagaggccaggccagtctccaaagcgcctaatctatctggcgtctaaactggactctggagtccctgacaggttcactggcagtggatcagggacagatttcacactgaaaatcagcagagtggaggctgaggatttgggaatttattattgctggcaaggtacacattttccgctcacgttcggtgctgggaccaagctggagctgaaacgg

(19) United States
(12) Patent Application Publication
(10) Pub. No.: US 2022/0265821 A1
(43) Pub. Date: Aug. 25, 2022

(54) COMPOSITION AND METHODS OF TARGETING THE PRE-B CELL RECEPTOR FOR THE TREATMENT OF LEUKEMIAS AND LYMPHOMAS

(71) Applicant: PASCAL BIOSCIENCES INC., Vancouver (CA)

(72) Inventors: Patrick W. GRAY, SEATTLE, WA (US); Larry W. TJOELKER, SEATTLE, WA (US); Christi L. WOOD, SEATTLE, WA (US)

(21) Appl. No.: 17/627,997

(22) PCT Filed: Jul. 17, 2020
(86) PCT No.: PCT/US20/42529

§ 371 Date: Jan. 18, 2022

Related U.S. Application Data
(60) Provisional application No. 62/876,435, filed on Jul. 19, 2019.

(51) Int. Cl. A61K 39/395; C07K 16/28; A61K 45/06; A61P 35/02

(52) U.S. Cl. CPC A61K 39/3955; C07K 16/2803; A61K 45/06; A61P 35/02; C07K 2317/565; C07K 2317/31; C07K 2317/515

(57) ABSTRACT

The present invention relates to antibodies that bind the pre-B cell receptor components VpreB and lambda-5, and compositions comprising such antibodies for use in diagnosing and eliminating pre-BCR-expressing leukemia and lymphoma cells. In one aspect, the present invention provides isolated antibodies or an antigen-binding fragment thereof capable of specifically binding to a SLC. The SLC is composed of two noncovalently-linked polypeptides, VpreB and lambda-5.

### CROSS REFERENCE TO RELATED APPLICATIONS

This application claims priority to U.S. Provisional Patent Application No. 62/876,435, filed Jul. 19, 2019, the contents of which are herein incorporated by reference in its entirety.

### FIELD OF INVENTION

The present invention relates to the identification of antibodies specific for the pre-B cell receptor (pre-BCR) and to methods of use of pre-BCR antibodies in the treatment of disease. In particular, the pre-BCR antibodies and the methods described herein are useful for the treatment of B cell precursor acute lymphoblastic leukemia (BCP-ALL), other leukemias, and lymphomas.

### INCORPORATION BY REFERENCE OF SEQUENCE LISTING SUBMITTED ELECTRONICALLY

Incorporated by reference in its entirety is a computer-readable nucleotide/amino acid sequence listing submitted concurrently herewith and identified as follows: ASCII text file named “20003_SeqListing_ST25.txt,” 100,136 bytes, created 17 Jul. 2020.

### BACKGROUND OF THE INVENTION

B cells develop from hematopoietic precursor cells of the bone marrow into a plasma cell in an ordered maturation and selection process through the stages of pro-B cell, pre-B cell, immature B cell, and mature B cell. The pre-BCR is assembled from immunoglobulin (Ig) heavy (IgM) and surrogate light chains (SLC) together with the signaling molecules Ig alpha and Ig beta, necessary for pre-BCR mediated signaling (Monroe, Nat. Rev. Immunol. 6:283, 2006). The SLC is composed of two noncovalently-linked polypeptides, VpreB and lambda-5. SLC expression has been found only in pro-B and pre-B cells but not in more mature, IgM surface positive B cells (Rolink et al., Cell 66:1081, 1991). In the more mature stages of B cell development, a B cell receptor composed of Ig lambda or Ig kappa light chain bound to Ig heavy chain is expressed as an antigen-recognizing receptor; these cells no longer express the SLC components. The functional cell surface assembly of the pre-BCR signals the successful recombination of the heavy chain, initiates a burst of pre-B cell proliferation, and sets the stage for cell cycle arrest and the recombination of Ig light chain-encoding genes (S. Herzog et al., Nat. Rev. Immunol. 9:195, 2009). In mouse mutants unable to express a pre-BCR, B-cell development is blocked at the pro-B cell stage of differentiation (Kitamura et al., Cell 69:823, 1992). Without a productively assembled pre-BCR, developing B cells are programmed to die.

B cell malignancies arise from arrested progression at various stages of B cell development (Sanchez-Beato et al., Blood 101:1220, 2009). B cell development arrested at the pre-B cell stage results in leukemia cells that express the pre-BCR (Kohrer et al., Leukemia 30:1246, 2016). Called BCP-ALL, this malignancy predominantly affects children but also occurs in adults. BCP-ALL is highly curable with 5-year survival rates approaching 90% in children (Hunger et al., N. Engl. J. Med. 373:1541, 2015), and 75-85% in adolescents and young adults. Survival of older adults is less successful, with overall survival rates of 35-55% in middle aged adults and under 30% in those over age 60 (Mohseni et al., Am J Blood Res 8:29, 2018). The standard frontline treatment for BCP-ALL consists of a regimen of traditional chemotherapy agents and may be followed by bone marrow transplant in high risk groups. Although the overall survival is high in children, the drawbacks of frontline treatment include severe toxicities that interrupt normal growth and development, cause cognitive deficits, and often precipitate secondary malignancies later in life (Nguyen et al., Leukemia 22:2142, 2008). Recent advances using target-specific therapies have changed the treatment paradigm for BCP-ALL patients not cured by frontline therapy. These include small molecule inhibitors of intracellular kinases such as BCR-ABL1, antibodies targeting cell surface antigens such as CD19, CD20, or CD22, and T cell-recruiting bispecific antibodies and chimeric antigen receptor (CAR)-T-cells (Rafei et al., Leuk Lymphoma 16:1, 2019). These treatments are often not curative and often have severe side effects. The potential for therapeutic efficacy of antibodies to pre-BCR has not been assessed in patients with BCP-ALL. Dedera et al. (US 2003/0215453) noted VpreB mRNA expression in various cell lines and cells.

Non-Hodgkin lymphomas (NHLs) can occur at any age and are often marked by lymph nodes that are larger than normal, fever, and weight loss. The many different types of NHL can be characterized as either aggressive (fast-growing) or indolent (slow-growing), and they can be formed from either B cell or T cell lineages. B cell NHLs include Burkitt lymphoma, chronic lymphocytic leukemia/small lymphocytic lymphoma, diffuse large B-cell lymphoma, follicular lymphoma, immunoblastic large cell lymphoma, precursor B-lymphoblastic lymphoma, and mantle cell lymphoma (Armitage et al., Lancet 390:298, 2017).

Thymoma is a rare neoplasm originating from thymic tissue and developing in the anterior mediastinal compartment. Early-stage thymoma treatment is surgery. However, radiation and chemotherapy are also widely applied as adjuvant and palliative treatment (Tomaszek et al., Ann. Thorac. Surg. 87:1973, 2009). For advanced thymoma, optimal treatment strategies have yet to be determined. There is an unmet medical need for new potent agents for the treatment of thymoma.

T cell acute lymphoblastic leukemia (T-ALL) arises from T lymphocyte-producing stem cells and is very aggressive. T-ALL accounts for approximately 20% of all cases of ALL and is somewhat more common in adults than children (Marks et al., Blood 114:5136, 2009). Although the overall survival for T-ALL has improved during the past 20 years, T-ALL remains the most difficult form of childhood ALL to treat (Goldberg et al., J. Clin. Oncol. 21:3616, 2003). Thus, there is a need for the development of improved therapies for treatment of T-ALL.

Acute myeloid leukemia (AML) is an aggressive hematological malignancy characterized by the accumulation of immature myeloid precursors. AML originates in the bone marrow and spreads to the bloodstream (Kavanagh et al., JCI Insight 2:1, 2017). AML is the most common acute leukemia in adults, with an incidence of over 20,000 cases per year in the United States alone (Siegel et al., CA Cancer J. Clin. 65:5, 2015). Despite recent progress, current treatment of AML remains unsatisfactory, with high rates of relapse after intensive therapy. Therefore, there remains a need for effective treatments for AML. Emerging immunological therapies for AML include monoclonal antibodies targeting CD33, CD123, and CD47, all of which are being evaluated in clinical trials (Kavanagh et al., JCI Insight 2:1, 2017). However, no antibody targeting VpreB or lambda-5 for AML has been disclosed in the literature.

Antibodies that target the VpreB and lambda-5 components of the pre-BCR have been previously generated (Lassoued et al., Cell 73:73, 1993; Geulpa-Fonlupt et al., Eur. J. Immunol. 24:257, 1994; Meffre et al., Eur. J. Immunol. 26:2172, 1996; Sanz et al., J. Exp. Med. 183:2693, 1996). Similarly, commercial antibodies for research purposes targeting either VpreB or lamba-5 of mouse and human are also readily available (e.g. Biolegend, VpreB Catalog #347404; Biolegend, lambda-5 Catalog #349803). Previous studies suggest that antibodies which bind pre-BCR on human BCP-ALL may be used to diagnose BCP-ALL patients (Tsuganezawa et al. U.S. Pat. No. 6,335,175 B1). The potential therapeutic utility of pre-BCR antibodies for BCP-ALL has been suggested (Tsuganezawa et al. U.S. Pat. No. 6,335,175 B1; van der Veer et al., Blood Cancer J. 4:181, 2014; Wilson et al., WO216127043 A1; Erasmus et al., Sci. Signal. 9:1, 2016) as has interfering with intracellular pre-BCR signaling using a kinase inhibitor (van der Veer et al., Blood Cancer J. 4:181, 2014). Inhibitors of BCR signaling have been introduced into patient care for various subtypes of mature B-cell lymphoma (e.g. ibrutinib, idelalisib; Muschen, Blood 125:3688, 2015).

However, potential clinical utility of VpreB and lambda-5 antibodies for the treatment of AML, T-ALL, thymoma, and B and T cell lymphomas has yet to be addressed. There is a need in the art for further therapeutic agents to treat such cancers such as antibodies that can target the pre-BCR in cancerous cells.

### BRIEF SUMMARY OF INVENTION

The present invention concerns antibodies specific for the pre-BCR and their uses.

In one aspect, the present invention provides isolated antibodies or an antigen-binding fragment thereof capable of specifically binding to a SLC. The SLC is composed of two noncovalently-linked polypeptides, VpreB and lambda-5. In one aspect, the present invention provides an isolated monoclonal antibody or antigen-binding fragment thereof that specifically binds to human VpreB (SEQ ID NO:1). In another aspect, the present invention provides an isolated monoclonal antibody or antigen-binding fragment thereof that specifically binds to human lambda-5 (SEQ ID NO:3).

In one aspect, the present invention provides an isolated monoclonal antibody or antigen-binding fragment thereof that specifically binds to mouse VpreB1 (SEQ ID NO:2).

Sequences
human VpreB sequence (with leader sequence
underlined)
SEQ ID NO: 1
MSWAPVLLML FVYCTGCGPQ PVLHQPPAMS SALGTTIRLT
CTLRNDHDIG VYSVYWYQQR PGHPPRFLLR YFSQSDKSQG
PQVPPRFSGS KDVARNRGYL SISELQPEDE AMYYCAMGAR
SSEKEERERE WEEEMEPTAA RTRVP
mouse VpreB1 sequence (with leader sequence
underlined)
SEQ ID NO: 2
MAWTSVLLML LAYLTGCGPQ PMVHQPPLAS SSLGATIRLS
CTLSNDHNIG IYSIYWYQQR PGHPPRFLLR YFSHSDKHQG
PDIPPRFSGS KDTTRNLGYL SISELQPEDE AVYYCAVGLR
SQEKKRMERE WEGEKSYTDL GS
human lambda-5 sequence (with leader sequence
underlined)
SEQ ID NO: 3
MRPGTGQGGL EAPGEPGPNL RQRWPLLLLG LAVVTHGLLR
PTAASQSRAL GPGAPGGSSR SSLRSRWGRF LLQRGSWTGP
RCWPRGFQSK HNSVTHVFGS GTQLTVLSQP KATPSVTLFP
PSSEELQANK ATLVCLMNDF YPGILTVTWK ADGTPITQGV
EMTTPSKQSN NKYAASSYLS LTPEQWRSRR SYSCQVMHEG
STVEKTVAPA ECS
human immunoglobulin G1 constant region
sequence
SEQ ID NO: 4
ASTKGPSVFP LAPSSKSTSG GTAALGCLVK DYFPEPVTVS
WNSGALTSGV HTFPAVLQSS GLYSLSSVVT VPSSSLGTQT
YICNVNHKPS NTKVDKKVEP KSCDKTHTCP PCPAPELLGG
PSVFLFPPKP KDTLMISRTP EVTCVVVDVS HEDPEVKFNW
YVDGVEVHNA KTKPREEQYN STYRVVSVLT VLHQDWLNGK
EYKCKVSNKA LPAPIEKTIS KAKGQPREPQ VYTLPPSRDE
LTKNQVSLTC LVKGFYPSDI AVEWESNGQP ENNYKTTPPV
LDSDGSFFLY SKLTVDKSRW QQGNVFSCSV MHEALHNHYT
QKSLSLSPG
human immunoglobulin kappa constant region
sequence
SEQ ID NO: 5
TVAAPSVFIF PPSDEQLKSG TASVVCLLNN FYPREAKVQW
KVDNALQSGN SQESVTEQDS KDSTYSLSST LTLSKADYEK
HKVYACEVTH QGLSSPVTKS FNRGEC

In another aspect, the present invention provides an isolated antibody, or antigen-binding fragment thereof, that binds to VpreB comprising: (a) a heavy chain (HC) variable region (VH) comprising a HC complementarity-determining region (CDR) 1 set forth as SEQ ID NO:19 (SYWMQ); a HC CDR2 SEQ ID NO:21 (EINPSNGRINYNEKFKS); and a HC CDR3 SEQ ID NO:23 (SGLLDY); and/or (b) a light chain (LC) variable region (VL) comprising a LC CDR1 set forth as SEQ ID NO:42 (RSSQSLIHSNGNTYLH); a LC CDR2 set forth as SEQ ID NO:44 (KVSNRFS); and a LC CDR3 set forth as SEQ ID NO:46 (SQSTYVPLT). [mAb 5-2D7]

In another aspect, the present invention provides an isolated antibody, or antigen-binding fragment thereof, that binds to VpreB comprising: (a) a VH comprising a HC CDR1 set forth as SEQ ID NO:19 (SYWMQ); a HC CDR2 SEQ ID NO:26 (EINPSNGRNNYNEKFKR); and a HC CDR3 SEQ ID NO:23 (SGLLDY); and/or (b) a VL comprising a LC CDR1 set forth as SEQ ID NO:48 (RSSQSLVHSNGNTYLH); a LC CDR2 set forth as SEQ ID NO:44 (KVSNRFS); and a LC CDR3 set forth as SEQ ID NO:46 (SQSTYVPLT). [mAb5-4A9]

In another aspect, the present invention provides an isolated antibody, or antigen-binding fragment thereof, that binds to VpreB comprising: (a) a VH comprising a HC CDR1 set forth as SEQ ID NO:30 (SDYWT); a HC CDR2 SEQ ID NO:32 (YISYSGRTYYNPSLKS); and a HC CDR3 SEQ ID NO:34 (ERYYYGSLDY); and/or (b) a VL comprising a LC CDR1 set forth as SEQ ID NO:48 (RSSQSLVHSNGNTYLH); a LC CDR2 set forth as SEQ ID NO:44 (KVSNRFS); and a LC CDR3 set forth as SEQ ID NO:53 (SQTTHVPPT). [mAb 5-9B12]

In another aspect, the present invention provides an isolated antibody, or antigen-binding fragment thereof, that binds to VpreB comprising: (a) a VH comprising a HC CDR1 set forth as SEQ ID NO:30 (SDYWT); a HC CDR2 SEQ ID NO:32 (YISYSGRTYYNPSLKS); and a HC CDR3 SEQ ID NO:34 (ERYYYGSLDY); and/or (b) a VL comprising a LC CDR1 set forth as SEQ ID NO:48 (RSSQSLVHSNGNTYLH); a LC CDR2 set forth as SEQ ID NO:44 (KVSNRFS); and a LC CDR3 set forth as SEQ ID NO:53 (SQTTHVPPT). [mAb 5-11D1]

In another aspect, the present invention provides an isolated antibody, or antigen-binding fragment thereof, that binds to VpreB comprising: (a) a VH comprising a HC CDR1 set forth as SEQ ID NO:30 (SDYWT); a HC CDR2 SEQ ID NO:37 (YISSSGRIYYNPSLKS); and a HC CDR3 SEQ ID NO:34 (ERYYYGSLDY); and/or (b) a VL comprising a LC CDR1 set forth as SEQ ID NO:55 (RSSQGLVHSNGNTYLH); a LC CDR2 set forth as SEQ ID NO:44 (KVSNRFS); and a LC CDR3 set forth as SEQ ID NO:53 (SQTTHVPPT). [mAb 5-14A8]

In another aspect, the present invention provides an isolated antibody, or antigen-binding fragment thereof, that binds to VpreB comprising: (a) a VH comprising a HC CDR1 set forth as SEQ ID NO:39 (SNWMN); a HC CDR2 SEQ ID NO:21 (EINPSNGRINYNEKFKS); and a HC CDR3 SEQ ID NO:23 (SGLLDY); and/or (b) a VL comprising a LC CDR1 set forth as SEQ ID NO:48 (RSSQSLVHSNGNTYLH); a LC CDR2 set forth as SEQ ID NO:44 (KVSNRFS); and a LC CDR3 set forth as SEQ ID NO:56 (SQSTYLPLT). [mAb5-14H5]

In another aspect, the present invention provides an isolated antibody, or antigen-binding fragment thereof, that binds to lambda-5 comprising (a) a VH comprising a HC CDR1 set forth as SEQ ID NO:79 (DYYLH); a HC CDR2 SEQ ID NO:81 (WIDPENGNTDYAPKFQG); and a HC CDR3 SEQ ID NO:83 (GYYDYDTDSAMDY); and/or (b) a VL comprising a LC CDR1 set forth as SEQ ID NO:86 (RSSQSLVHSDGITYLH); a LC CDR2 set forth as SEQ ID NO:88 (KVSNRFS); and a LC CDR3 set forth as SEQ ID NO:90 (SQSTRVPWT). [mAb 4-15E6]

In another aspect, the present invention provides an isolated antibody, or antigen-binding fragment thereof, that binds to lambda-5 comprising (a) a VH comprising a HC CDR1 set forth as SEQ ID NO:115 (NYWMH); a HC CDR2 SEQ ID NO:123 (AIYPGNSDTSYNQKFKG); and a HC CDR3 SEQ ID NO:131 (ADYDGTPFDY); and/or (b) a VL comprising a LC CDR1 set forth as SEQ ID NO:143 (KSSQSLLDSDGETYLS); a LC CDR2 set forth as SEQ ID NO:154 (LVSKLDS); and a LC CDR3 set forth as SEQ ID NO:159 (WQGTHFPLT). [mAb 4-6D12]

In another aspect, the present invention provides an isolated antibody, or antigen-binding fragment thereof, that binds to lambda-5 comprising (a) a VH comprising a HC CDR1 set forth as SEQ ID NO:117 (SYWMH); a HC CDR2 SEQ ID NO:124 (AIYPGSSDTSYSQKFKG); and a HC CDR3 SEQ ID NO:133 (GDYDGTPFDY); and/or (b) a VL comprising a LC CDR1 set forth as SEQ ID NO:145 (KSGQSLLDSDGKTYLN); a LC CDR2 set forth as SEQ ID NO:156 (LVSKLHS); and a LC CDR3 set forth as SEQ ID NO:159 (WQGTHFPLT). [mAb 4-5G11]

In another aspect, the present invention provides an isolated antibody, or antigen-binding fragment thereof, that binds to lambda-5 comprising (a) a VH comprising a HC CDR1 set forth as SEQ ID NO:117 (SYWMH); a HC CDR2 SEQ ID NO:125 (AIYLGNTDTSYNQKFKG); and a HC CDR3 SEQ ID NO:131 (ADYDGTPFDY); and/or (b) a VL comprising a LC CDR1 set forth as SEQ ID NO:147 (RSSQSLLDSDGETYLS); a LC CDR2 set forth as SEQ ID NO:154 (LVSKLDS); and a LC CDR3 set forth as SEQ ID NO:159 (WQGTHFPLT). [mAb 4-7A6]

In another aspect, the present invention provides an isolated antibody, or antigen-binding fragment thereof, that binds to lambda-5 comprising (a) a VH comprising a HC CDR1 set forth as SEQ ID NO:117 (SYWMH); a HC CDR2 SEQ ID NO:123 (AIYPGNSDTSYNQKFKG); and a HC CDR3 SEQ ID NO:131 (ADYDGTPFDY); and/or (b) a VL comprising a LC CDR1 set forth as SEQ ID NO:143 (KSSQSLLDSDGETYLS); a LC CDR2 set forth as SEQ ID NO:154 (LVSKLDS); and a LC CDR3 set forth as SEQ ID NO:159 (WQGTHFPLT). [mAb 4-7C1]

In another aspect, the present invention provides an isolated antibody, or antigen-binding fragment thereof, that binds to lambda-5 comprising (a) a VH comprising a HC CDR1 set forth as SEQ ID NO:117 (SYWMH); a HC CDR2 SEQ ID NO:124 (AIYPGSSDTSYSQKFKG); and a HC CDR3 SEQ ID NO:133 (GDYDGTPFDY); and/or (b) a VL comprising a LC CDR1 set forth as SEQ ID NO:145 (KSGQSLLDSDGKTYLN); a LC CDR2 set forth as SEQ ID NO:156 (LVSKLHS); and a LC CDR3 set forth as SEQ ID NO:159 (WQGTHFPLT). [mAb 4-9H8]

In another aspect, the present invention provides an isolated antibody, or antigen-binding fragment thereof, that binds to lambda-5 comprising (a) a VH comprising a HC CDR1 set forth as SEQ ID NO:79 (DYYLH); a HC CDR2 SEQ ID NO:127 (WIDPENGATDYAPKFQG); and a HC CDR3 SEQ ID NO:137 (GYYDYDADSAMDY); and/or (b) a VL comprising a LC CDR1 set forth as SEQ ID NO:86 (RSSQSLVHSDGITYLH); a LC CDR2 set forth as SEQ ID NO:88 (KVSNRFS); and a LC CDR3 set forth as SEQ ID NO:160 (SQSARVPWT). [mAb 4-12G1]

In another aspect, the present invention provides an isolated antibody, or antigen-binding fragment thereof, that binds to lambda-5 comprising (a) a VH comprising a HC CDR1 set forth as SEQ ID NO:115 (NYWMH); a HC CDR2 SEQ ID NO:128 (AIYPGNSDTSYNQNFKG); and a HC CDR3 SEQ ID NO:131 (ADYDGTPFDY); and/or (b) a VL comprising a LC CDR1 set forth as SEQ ID NO:143 (KSSQSLLDSDGETYLS); a LC CDR2 set forth as SEQ ID NO:154 (LVSKLDS); and a LC CDR3 set forth as SEQ ID NO:159 (WQGTHFPLT). [mAb 4-17G9]

In another aspect, the present invention provides an isolated antibody, or antigen-binding fragment thereof, that binds to lambda-5 comprising (a) a VH comprising a HC CDR1 set forth as SEQ ID NO:115 (NYWMH); a HC CDR2 SEQ ID NO:129 (AVYPGNSDTSYSQKFTG); and a HC CDR3 SEQ ID NO:131 (ADYDGTPFDY); and/or (b) a VL comprising a LC CDR1 set forth as SEQ ID NO:143 (KSSQSLLDSDGETYLS); a LC CDR2 set forth as SEQ ID NO:154 (LVSKLDS); and a LC CDR3 set forth as SEQ ID NO:159 (WQGTHFPLT). [mAb 4-18G6]

In another aspect, the present invention provides an isolated antibody, or antigen-binding fragment thereof, that binds to lambda-5 comprising (a) a VH comprising a HC CDR1 set forth as SEQ ID NO:117 (SYWMH); a HC CDR2 SEQ ID NO:123 (AIYPGNSDTSYNQKFKG); and a HC CDR3 SEQ ID NO:131 (ADYDGTPFDY); and/or (b) a VL comprising a LC CDR1 set forth as SEQ ID NO:143 (KSSQSLLDSDGETYLS); a LC CDR2 set forth as SEQ ID NO:154 (LVSKLDS); and a LC CDR3 set forth as SEQ ID NO:159 (WQGTHFPLT). [mAb 4-19A9]

In another aspect, the present invention provides an isolated antibody, or antigen-binding fragment thereof, that binds to lambda-5 comprising (a) a VH comprising a HC CDR1 set forth as SEQ ID NO:117 (SYWMH); a HC CDR2 SEQ ID NO:123 (AIYPGNSDTSYNQKFKG); and a HC CDR3 SEQ ID NO:133 (GDYDGTPFDY); and/or (b) a VL comprising a LC CDR1 set forth as SEQ ID NO:152 (KSSQSLLDSDGETYLN); a LC CDR2 set forth as SEQ ID NO:157 (LASKLDS); and a LC CDR3 set forth as SEQ ID NO:159 (WQGTHFPLT). [mAb 4-20D2]

In yet another aspect, the CDRs disclosed herein include consensus sequences derived from groups of related monoclonal antibodies. As described herein, a “consensus sequence” refers to amino acid sequences having common conserved amino acids and one or more variable amino acids are specified. The CDR consensus sequences provided include CDRs corresponding to each of CDRH1, CDRH2, CDRH3, CDRL1, CDRL2 and CDRL3. Each of the consensus CDR sequences can be combined with any representative of the other five types of CDRs described herein.

Thus, the present invention also provides an isolated antibody, or antigen-binding fragment thereof, that binds to VpreB comprising (a) a VH comprising HC CDR1 of SEQ ID NO: 58, HC CDR 2 of SEQ ID NO: 59, and CDR3 of SEQ ID NO: 23 as described herein, and/or (b) a VL comprising LC CDR1 of SEQ ID NO: 60, CDR2 of SEQ ID NO: 44, and LC CDR3 of SEQ ID NO: 61 as described herein. [VpreB group IA]

The present invention also provides an isolated antibody, or antigen-binding fragment thereof, that binds to VpreB comprising (a) VH comprising HC CDR1 of SEQ ID NO: 30, HC CDR2 of SEQ ID NO: 62, and HC CDR3 of SEQ ID NO: 34 as described herein, and/or (b) a VL comprising LC CDR1 of SEQ ID NO: 63, CDR2 of SEQ ID NO: 44, LC CDR3 of SEQ ID NO: 53 as described herein. [VpreB group TB]

The present invention also provides an isolated antibody, or antigen-binding fragment thereof, that binds to lambda-5 comprising (a) a VH comprising HC CDR1 of SEQ ID NO: 164, HC CDR2 of SEQ ID NO: 165, and HC CDR3 of SEQ ID NO: 166 as described herein, and/or (b) a VL comprising LC CDR1 of SEQ ID NO: 167, LC CDR2 of SEQ ID NO: 168, and LC CDR3 of SEQ ID NO: 159 as described herein. [Lambda-5 group IA]

The present invention also provides an isolated antibody, or antigen-binding fragment thereof, that binds to lambda-5 comprising (a) a VH comprising HC CDR1 of SEQ ID NO: 79, HC CDR2 of SEQ ID NO: 169, and HC CDR3 of SEQ ID NO: 170 described herein, and/or (b) a VL comprising LC CDR1 of SEQ ID NO: 86, LC CDR2 of SEQ ID NO: 88, and LC CDR3 of SEQ ID NO: 171 as described herein. [Lambda-5 group TB]

Any of the preceding antibodies, or antigen-binding fragments thereof, may comprise both the VL and the VH recited. Example antibodies include chimeric, human or humanized antibodies, including IgG, or IgG1, IgG2, IgG3 or IgG4. Example antigen-binding fragments include VL, VH, Fab, Fab′, F(ab′)2, scFv, or (scFv)2 fragment.

This invention also contemplates the use of conjugates comprising (1) a cell-binding agent, any of the antibodies or antibody fragments described herein, that recognizes and binds VpreB or lamba-5, and (2) a cytotoxic agent (antibody-drug conjugate (ADC)). In the cytotoxic conjugates, the cell binding agent (antibody or antibody fragment) has a high affinity for VpreB or lamba-5 and the cytotoxic agent has a high degree of cytotoxicity for cells expressing VpreB or lamba-5, such that the cytotoxic conjugates of the present invention form effective killing agents. For example, the antibody or antigen-binding fragment thereof has an affinity for VpreB of about 10⁻⁷M or less, or about 10⁻⁸M or less, or about 10⁻⁹M or less, or about 10⁻¹⁰ M or less, or about 10⁻¹¹ M or less, or about 10⁻¹²M or less. For example, the antibody or antigen-binding fragment thereof has an affinity for lambda-5 of about 10⁻⁷M or less, or about 10⁻⁸M or less, or about 10⁻⁹M or less, or about 10⁻¹⁰ M or less, or about 10⁻¹¹ M or less, or about 10⁻¹²M or less. In any of these aspects, the antibody or antigen-binding fragment may bind to and promote internalization of pre-BCR.

Antibodies or antibody fragments that bind to the same epitope as, or that cross-compete with, any of the antibodies disclosed herein are also provided.

The term “binding agent” is used interchangeably with antibody or antibody fragment.

In some aspects, the VpreB-binding agent (antibody or antibody fragment) has a cytotoxic, cytostatic and/or immunomodulatory effect on VpreB-expressing cells. Such an effect can be mediated, for example, by the depletion or inhibition of the proliferation or differentiation of VpreB-expressing cells. In some embodiments, the VpreB-binding agent can mediate effector function. In some embodiments, the VpreB-binding agent is conjugated to a therapeutic agent (e.g., an ADC). In other embodiments, the VpreB-binding agent is unconjugated, for example, not conjugated to a therapeutic agent (e.g., a VpreB naked antibody). In other embodiments, the VpreB-binding agent is a bispecific antibody, or a multispecific antibody.

In some aspects, the lambda-5-binding agent (antibody or antibody fragment) has a cytotoxic, cytostatic and/or immunomodulatory effect on lambda-5-expressing cells. Such an effect can be mediated, for example, by the depletion or inhibition of the proliferation or differentiation of lambda-5-expressing cells. In some embodiments, the lambda-5-binding agent can mediate effector function. In some embodiments, the lambda-5-binding agent is conjugated to a therapeutic agent (e.g., an ADC). In other embodiments, the lambda-5-binding agent is unconjugated, for example, not conjugated to a therapeutic agent (e.g. a lambda-5 naked antibody). In other embodiments, the lamba-5-binding agent is a bispecific antibody, or a multispecific antibody.

Another aspect of the present invention contemplates making a CAR-T comprising transducing a T-cell or NK cell with the polynucleotide encoding any of the antibodies or antibody fragments described herein under suitable conditions, or substituting any of the CDRs described herein into a T cell receptor.

The present invention also contemplates nucleic acids encoding the antibodies, or antigen-binding fragments thereof, or VH, or VL, of the invention, vectors comprising such nucleic acids, preferably operably linked to a heterologous expression control sequence, host cells comprising such nucleic acids or vectors, and methods of producing antibodies, or antigen-binding fragments thereof. Such methods include culturing the host cell in culture medium under conditions and for a time period suitable for expressing the antibody or antigen-binding fragment thereof, and recovering the antibody or antigen-binding fragment from the host cell or culture medium. Immune cells, including T cells or NK cells, that comprise the nucleic acids and express the antibody or antigen-binding fragment on their surface, are also contemplated.

In another aspect, the present invention describes a pharmaceutical composition that includes any embodiment of the composition summarized above and a pharmaceutically acceptable carrier.

In a further embodiment, the present invention comprises pharmaceutical compositions comprising an antibody, epitope-binding fragment thereof, or immunoconjugate of the present invention, either alone or in combination with a drug or prodrug or other therapeutic agent, in the presence of one or more pharmaceutically acceptable agent.

Also provided by the present invention is the use of a VpreB- or lambda-5-binding agent in the manufacture of a medicament for the killing or inhibition of the proliferation or differentiation of VpreB- or lambda-5-expressing cells. In some embodiments, a VpreB or lambda-5 full length antibody or antigen-binding fragment thereof or derivative thereof that is not conjugated to a cytotoxic, cytostatic and/or therapeutic agent will be used. In some other embodiments, a ligand-drug conjugate (e.g., a VpreB- or lambda-5-binding agent such as a full length antibody or antigen-binding fragment thereof or derivative thereof conjugated to a cytotoxic, cytostatic and/or therapeutic agent) will be used.

In another aspect, this disclosure describes a method of treating cancer. Generally, the method includes administering to a subject having cancer any embodiment of the pharmaceutical compositions summarized above in an amount effective to ameliorate at least one symptom or clinical sign of cancer. In some embodiments, the cancer is a hematologic cancer. In some embodiments, the cancer is leukemia, lymphoma, or myeloma.

In a further embodiment, the invention provides methods for the treatment of a subject having a disease wherein VpreB is expressed comprising administering an antibody, an epitope-binding fragment thereof, or immunoconjugate of the present invention, either alone or in combination with another drug or prodrug or another therapeutic agent, further alone or in the presence of one or more pharmaceutically acceptable agents. The disease may be one or more of, for example, B cell lineage malignancies such as, for example, B cell lymphomas or B cell leukemias, including, but not limited to, NHL, and acute lymphoblastic leukemia (ALL). NHL and other cancers may include AML, T-ALL, thymoma, lymphoma, mantel cell lymphoma (MCL), marginal zone lymphoma (MZL), diffuse large B cell lymphoma (DLBCL), follicular lymphoma (FL), Waldenstrom macroglobulinemia (WM), multiple myeloma (MM), or other diseases yet to be determined in which VpreB is expressed. Other diseases may include autoimmune or immune-mediated inflammatory disease (especially inflammatory bowel disease, Crohn's disease, ulcerative colitis, multiple sclerosis, psoriasis, rheumatoid arthritis, systemic lupus erythematosus, type 1 diabetes, vasculitis, asthma, eczema and atopic dermatitis, fibrosis, graft rejection, and graft-versus-host-disease).

In a further embodiment, the invention provides methods for the treatment of a subject having a disease wherein lambda-5 is expressed comprising administering an antibody, an epitope-binding fragment thereof, or immunoconjugate of the present invention, either alone or in combination with another drug or prodrug or another therapeutic agent, further alone or in the presence of one or more pharmaceutically acceptable agents. The disease may be one or more of, for example, B cell lineage malignancies such as, for example, B cell lymphomas or B cell leukemias, including, but not limited to, NHL, and ALL. NHL and other cancers may include AML, T-ALL, thymoma, lymphoma, MCL, MZL, DLBCL, FL, WM, and MM or other diseases yet to be determined in which lambda-5 is expressed. Other diseases may include autoimmune or immune-mediated inflammatory disease (especially inflammatory bowel disease, ulcerative colitis, Crohn's disease, multiple sclerosis, psoriasis, rheumatoid arthritis, systemic lupus erythematosus, type 1 diabetes, vasculitis, asthma, eczema and atopic dermatitis, fibrosis, graft rejection, and graft-versus-host-disease).

In another aspect, the invention also includes diagnostic use of a pre-BCR antibody. For example, the pre-BCR antibody can be used as a diagnostic imaging agent alone and/or in combination with other diagnostic imaging agents and/or in conjunction with therapeutic applications. The diagnostic agent can be used in vivo in human patients known to have or have had a pre-BCR-associated disorder.

In another aspect, the diagnostic test can be used to identify patients with a pre-BCR-associated disorder, or to determine the extent of such a disorder in a particular patient, or to monitor the course of a disorder over time, or the effect of a chosen treatment on a disorder.

In another aspect, the present invention provides an isolated monoclonal antibody or antigen-binding fragment thereof that specifically binds to the pre-BCR of companion animals, such as dogs and cats.

In another aspect, the present invention provides a method of treating a lymphoma in a dog comprising administering to a dog or cat in need of such treatment a therapeutically effective amount of antibody, an epitope-binding fragment thereof or immunoconjugate of the present invention, either alone or in combination with another drug or prodrug or another therapeutic agent, further alone or in the presence of one or more pharmaceutically acceptable agents.

The antibody compounds of the present disclosure can be used as medicaments in human and veterinary medicine. Veterinary applications include the treatment of companion/pet animals, such as cats and dogs; working animals, such as guide or service dogs, and horses; sport animals, such as horses and dogs; zoo animals, such as primates, cats such as lions and tigers, bears, etc.; and other valuable animals kept in captivity.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The foregoing aspects and many of the attendant advantages of this invention will become more readily appreciated as the same become better understood by reference to the following detailed description, when taken in conjunction with the accompanying drawings, wherein:

FIG. 1A illustrates the expression of human VPREB1 (VpreB) mRNA in multiple karyotypically distinct BCP-ALL specimens. Chronic lymphocytic leukemia (CLL) and healthy bone marrow represent non-BCP-ALL control examples.

FIG. 1B illustrates the expression of human IGLL1 (lambda-5) mRNA in multiple karyotypically distinct BCP-ALL specimens. CLL and healthy bone marrow represent non-BCP-ALL control examples.

FIG. 2A illustrates the expression of human VPREB1 (VpreB) in multiple types of cancer.

FIG. 2B illustrates the expression of human IGLL1 (lambda-5) mRNA in multiple types of cancer.

FIG. 3A illustrates the expression of human VPREB1 (VpreB) mRNA in multiple karyotypically distinct AML specimens. Healthy hematopoietic cell types (HSC: hematopoietic stem cell; CMP: common myeloid progenitor cell; PMN: polymorphonuclear cells; Mono: monocytes) and a single BCP-ALL subtype represent non-AML control examples.

FIG. 3B illustrates the expression of human IGLL1 (lambda-5) mRNA in multiple karyotypically distinct AML specimens. Healthy hematopoietic cell types (HSC, CMP, PMN, Mono) and a single BCP-ALL subtype represent non-AML control examples.

FIG. 4A depicts human VPREB1 (VpreB) mRNA expression in T-ALL compared to two karyotypically distinct BCP-ALL subtypes, with CLL and healthy bone marrow included as non-ALL control examples.

FIG. 4B depicts human IGLL1 (lambda-5) mRNA expression in T-ALL compared to two karyotypically distinct BCP-ALL subtypes, with CLL and healthy bone marrow included as non-ALL control examples.

FIG. 5A illustrates human VPREB1 (VpreB) mRNA expression in cancer cell lines representing certain leukemias, lymphomas, and solid cancers.

FIG. 5B illustrates human IGLL1 (lambda-5) mRNA expression in cancer cell lines representing certain leukemias, lymphomas, and solid cancers.

FIG. 6A is an amino acid sequence alignment of the VH regions of the VpreB antibodies 5-2D7 (SEQ ID NO:6), 5-4A9 (SEQ ID NO:7), 5-9B12 (SEQ ID NO:8), 5-11D1 (SEQ ID NO:9), 5-14A8 (SEQ ID NO:10), and 5-14H5 (SEQ ID NO:11). Kabat CDRs are underlined.

FIG. 6B is an amino acid sequence alignment of the VL regions of the VpreB antibodies 5-2D7 (SEQ ID NO:12), 5-4A9 (SEQ ID NO:13), 5-9B12 (SEQ ID NO:14), 5-11D1 (SEQ ID NO:15), 5-14A8 (SEQ ID NO:16), and 5-14H5 (SEQ ID NO:17). Kabat CDRs are underlined.

FIG. 7 schematically depicts the degree of amino acid identity between the Ig lambda-like region of human VpreB and a consensus human Ig lambda V region sequence, and the degree of amino acid identity between the Ig lambda-like region of human lambda-5 and a consensus human Ig lambda constant region sequence. Unique region (UR) sequences bear no resemblance to Ig lambda.

FIG. 8A shows the flow cytometry results of VpreB antibodies 5-2D7, 5-4A9, 5-9B12, 5-11D1, 5-14A8, and 5-14H5 binding to the human SLC-expressing pre-B cell line, NALM-6, as described in Example 4.

FIG. 8B shows the flow cytometry results of VpreB antibodies 5-2D7, 5-4A9, 5-9B12, 5-11D1, 5-14A8, and 5-14H5 binding to the human Ig lambda-expressing B cell line, Ramos, as described in Example 4.

FIG. 8C shows the flow cytometry results of VpreB antibodies 5-2D7, 5-4A9, 5-9B12, 5-11D1, 5-14A8, and 5-14H5 binding to the human Ig kappa-expressing B cell line, Raji, as described in Example 4.

FIG. 8D shows the flow cytometry results of VpreB antibodies 5-4A9, 5-11D1, 5-14A8, and 5-14H5 binding to the mouse pre-B cell line, L1.2, as described in Example 4.

FIG. 9 depicts an alignment between human VpreB (SEQ ID NO:1) and mouse VpreB1 (SEQ ID NO:2) protein sequences.

FIG. 10 shows the flow cytometry results of VpreB antibodies 5-2D7, 5-4A9, 5-9B12, 5-11D1, 5-14A8, and 5-14H5 binding to the human colorectal cancer cell line, COLO 205, as described in Example 4.

FIG. 11 shows the flow cytometry results of VpreB antibodies 5-2D7, 5-4A9, 5-9B12, 5-11D1, 5-14A8, and 5-14H5 binding to the human immortalized T cell line, Jurkat, as described in Example 4.

FIG. 12 shows the flow cytometry results of the lambda-5 antibody 4-15E6 binding to the human pre-B cell line NALM6, the human B cell lines Ramos and Raji, the human T cell line Jurkat, the human erythroleukemia cell line K562, and the human colorectal cancer cell line COLO 205 as described in Example 5.

FIG. 13 graphically illustrates saturation binding kinetics analysis of VpreB antibodies 5-4A9, 5-11D1, 5-14A8, and 5-14H5 on NALM-6 cells as described in Example 4. MFI: median fluorescence intensity.

FIG. 14A is an amino acid sequence alignment of the VH regions of the lambda-5 antibodies 4-6D12 (SEQ ID NO:94), 4-15E6 (SEQ ID NO:76), 4-5G11 (SEQ ID NO:95), 4-7A6 (SEQ ID NO:96), 4-7C1 (SEQ ID NO:97), 4-9H8 (SEQ ID NO:98), 4-12G1 (SEQ ID NO:99), 4-17G9 (SEQ ID NO:100), 4-18G6 (SEQ ID NO:101), 4-19A9 (SEQ ID NO:102), and 4-20D2 (SEQ ID NO:103). Kabat CDRs are underlined.

FIG. 14B is an amino acid sequence alignment of the VL regions of the lambda-5 antibodies 4-6D12 (SEQ ID NO:104), 4-15E6 (SEQ ID NO:77), 4-5G11 (SEQ ID NO:105), 4-7A6 (SEQ ID NO:106), 4-7C1 (SEQ ID NO:107), 4-9H8 (SEQ ID NO:108), 4-12G1 (SEQ ID NO:109), 4-17G9 (SEQ ID NO:110), 4-18G6 (SEQ ID NO:111), 4-19A9 (SEQ ID NO:112), and 4-20D2 (SEQ ID NO:113). Kabat CDRs are underlined.

FIG. 15 graphically illustrates saturation binding kinetics analysis of VpreB antibodies 5-2D7 and 5-9B21 on NALM-6 cells as described in Example 4. MFI: median fluorescence intensity.

FIG. 16 graphically illustrates saturation binding kinetics analysis of lambda-5 antibodies 4-6D12, 4-5G11, 4-7A6, 4-7C1, 4-9H8, 4-12G1, 4-15E6, 4-17G9, 4-18G6, 4- 19A9, and 4-20D2 on NALM-6 cells as described in Example 5. MFI: median fluorescence intensity.

FIG. 17 shows that the VpreB mAbs 5-4A9, 5-2D7, 5-9B12, 5-11D1, 5-14H5 and 5-14A8 compete for binding to NALM-6 cells. Antibody designated above each graph indicates the biotinylated mAb incubated with each of the nonbiotinylated mAbs represented by the titration curves graphically displayed. MFI: median fluorescence intensity.

FIG. 18 shows the flow cytometry results of the lambda-5 antibody 4-6D12 binding to the human pre-B cell line NALM6, the human B cell lines Ramos and Raji, the human T cell line Jurkat, the human erythroleukemia cell line K562, the human colorectal cancer cell line COLO 205, and the human embryonic kidney cell line tsA201 as described in Example 7.

FIG. 19 shows the flow cytometry results of the lambda-5 antibody 4-5G11 binding to the human pre-B cell line NALM6, the human B cell lines Ramos and Raji, the human T cell line Jurkat, the human erythroleukemia cell line K562, the human colorectal cancer cell line COLO 205, and the human embryonic kidney cell line tsA201 as described in Example 7.

FIG. 20 shows the flow cytometry results of the lambda-5 antibody 4-7A6 binding to the human pre-B cell line NALM6, the human B cell lines Ramos and Raji, the human T cell line Jurkat, the human erythroleukemia cell line K562, the human colorectal cancer cell line COLO 205, and the human embryonic kidney cell line tsA201 as described in Example 7.

FIG. 21 shows the flow cytometry results of the lambda-5 antibody 4-7C1 binding to the human pre-B cell line NALM6, the human B cell lines Ramos and Raji, the human T cell line Jurkat, the human erythroleukemia cell line K562, the human colorectal cancer cell line COLO 205, and the human embryonic kidney cell line tsA201 as described in Example 7.

FIG. 22 shows the flow cytometry results of the lambda-5 antibody 4-9H8 binding to the human pre-B cell line NALM6, the human B cell lines Ramos and Raji, the human T cell line Jurkat, the human erythroleukemia cell line K562, the human colorectal cancer cell line COLO 205, and the human embryonic kidney cell line tsA201 as described in Example 7.

FIG. 23 shows the flow cytometry results of the lambda-5 antibody 4-12G1 binding to the human pre-B cell line NALM6, the human B cell lines Ramos and Raji, the human T cell line Jurkat, the human erythroleukemia cell line K562, the human colorectal cancer cell line COLO 205, and the human embryonic kidney cell line tsA201 as described in Example 7.

FIG. 24 shows the flow cytometry results of the lambda-5 antibody 4-15E8 binding to the human embryonic kidney cell line tsA201 as described in Example 7.

FIG. 25 shows the flow cytometry results of the lambda-5 antibody 4-17G9 binding to the human pre-B cell line NALM6, the human B cell lines Ramos and Raji, the human T cell line Jurkat, the human erythroleukemia cell line K562, the human colorectal cancer cell line COLO 205, and the human embryonic kidney cell line tsA201 as described in Example 7.

FIG. 26 shows the flow cytometry results of the lambda-5 antibody 4-18G6 binding to the human pre-B cell line NALM6, the human B cell lines Ramos and Raji, the human T cell line Jurkat, the human erythroleukemia cell line K562, the human colorectal cancer cell line COLO 205, and the human embryonic kidney cell line tsA201 as described in Example 7.

FIG. 27 shows the flow cytometry results of the lambda-5 antibody 4-19A9 binding to the human pre-B cell line NALM6, the human B cell lines Ramos and Raji, the human T cell line Jurkat, the human erythroleukemia cell line K562, the human colorectal cancer cell line COLO 205, and the human embryonic kidney cell line tsA201 as described in Example 7.

FIG. 28 shows the flow cytometry results of the lambda-5 antibody 4-20D2 binding to the human pre-B cell line NALM6, the human B cell lines Ramos and Raji, the human T cell line Jurkat, the human erythroleukemia cell line K562, the human colorectal cancer cell line COLO 205, and the human embryonic kidney cell line tsA201 as described in Example 7.

FIG. 29 graphically illustrates the internalization of the VpreB mAbs 5-4A9, 5-9B12, 5-14A8, 5-2D7, 5-11D1, and 5-14H5 into NALM-6 cells over time.

### DETAILED DESCRIPTION

The invention described herein capitalizes on the restricted expression of the pre-BCR in the earliest stages of B cell development. Any agent targeting a pre-BCR-expressing malignancy might also impact normal pre-B cells but would not bind to more mature B cells, thus sparing the humoral branch of the immune system. In contrast, immunotherapies currently approved for B cell malignancies target CD19, CD20, and CD22, all of which are expressed on early stage as well as more mature B lineage cells. Humoral immunity is severely compromised in patients treated with these agents (Huguet and Tavitian, Exp. Opin. Emerging Drugs, 22:107, 2017).

Furthermore, this invention discloses VpreB and lambda-5 antibodies engineered to bind the pre-BCR, block signaling, recruit effector T cells as a bispecific antibody, or deliver a payload via an antibody drug conjugate.

Antibodies

As used herein, the term “antibody” refers to any portion of an immunoglobulin capable of specifically binding to a particular target. Thus, in some embodiments, the antibody can be an antibody fragment such as, for example, a monovalent form of the antibody (Fab-Fc), multispecific antibodies (e.g. bispecific antibodies) or an intact antibody conjugated to a toxin so long as they exhibit the desired biological activity. Antibodies may be murine, human, humanized, chimeric, camelid, or derived from other species. Once an antibody is identified, the antibody may be produced by any suitable means including, for example, recombinant techniques, synthetic techniques, expression from a hybridoma, and/or chemical modification of a monoclonal antibody produced by a hybridoma.

The antibodies can be immunoglobulins of any isotype (e.g., IgG, IgE, IgM, IgD, IgA and IgY), class (e.g., IgG1, IgG2, IgG3, IgG4, IgA1 and IgA2) or subclass. Each heavy chain comprises a variable region (VH) and at least a portion of a constant region. In immunoglobulins, the CH comprises three domains, CH1, CH2 and CH3. Each light chain comprises a variable region (VL) and at least a portion of a constant region (CL). The VH and VL regions can be further subdivided into regions of hypervariability, called complementarity determining regions (CDR), which are flanked by more conserved regions, called framework regions (FR).

Each VH and VL is composed of three CDRs and four FR's arranged from amino-terminus to carboxy-terminus in the following order: FR1, CDR1, FR2, CDR2, FR3, CDR3, and FR4. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

The variable regions of the heavy and light chains interact with antigen. An “epitope” of an antigen is any three-dimensional region or linear sequence of the antigen which is specifically recognized by an antibody or antibody fragment. Epitope mapping techniques are well known in the art and include hydrogen/deuterium exchange, x-ray crystallography and two-dimensional nuclear magnetic resonance.

The constant regions of the antibodies may mediate the binding of the immunoglobulin to host tissues or factors, including FcγR-expressing cells, resulting in phagocytosis or antibody-dependent cell-mediated cytotoxicity (ADCC), and binding to the first component (Clq) of the classical complement system, resulting in complement activation. The Fc-regions also contain a binding epitope for the neonatal Fc receptor (FcRn), responsible for the extended half-life, placental transport, and bidirectional transport of IgG to mucosal surfaces. Different subclasses have different effector functions; for example, IgG1 and IgG3 can mediate ADCC.

The term “monoclonal antibody” as used herein refers to a preparation of antibody molecules of substantially a single molecular composition.

The term “antibody fragment” or “antigen-binding fragment” of an antibody, as used herein, refers to one or more portions of an antibody that retain the ability to specifically bind an epitope of an antigen. Examples of binding fragments include, but are not limited to, a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a single chain Fv (ScFv) in which the VL and VH are joined by a linker; a dAb fragment (Ward et al., (1989) Nature 341:544-546), which consists of a VH domain; and an isolated complementarity determining region (CDR). Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) or a T-cell receptor (TCR). Antibody fragments can be incorporated into single chain molecules comprising a pair of tandem Fv segments such as VH-CH1-VH-CH1. which, together with complementary light chain polypeptides, form a pair of antigen-binding sites (Zapata et al., (1995) Protein Eng. 8:1057-1062.

Chimeric, CDR-grafted, and humanized antibodies based upon the CDRs described herein can readily be generated. A “CDR-grafted” antibody comprises one or more CDRs from one species (e.g., a rodent antibody) and frameworks from another species (e.g., a human framework, including a human consensus framework). See, e.g., Jones et al., 1986, Nature 321:522-525; Riechmann et al., 1988, Nature 332:323-27; Verhoeyen et al., 1988, Science 239:1534-1536.

A “humanized antibody” or “humanized antibody fragment,” as used herein, is an antibody molecule in which the six CDR sequences are primarily derived from a non-human species, while the remaining framework, and optionally the constant antibody regions, are primarily derived from sequences of human origin. In one example, humanization of a rodent antibody involves substitution of the rodent CDR sequences into a human framework, and sometimes modification of 1, 2, 3 or more residues of the framework to be more homologous to the original rodent framework. Sometimes modifications of 1, 2, 3 or more residues of the CDRs is necessary to increase affinity to the desired affinity.

A “human antibody” or “human antibody fragment”, as used herein, includes antibodies and antibody fragments having variable regions in which both the framework and CDR regions are derived from sequences of human origin. Furthermore, if the antibody contains a constant region, the constant region is preferably derived from human sequences. Human origin includes, e.g., human germline sequences in rodents, mutated versions of human germline sequences or antibody containing consensus framework sequences derived from human framework sequences analysis, and antibodies selected via phage display from libraries of human antibodies. Introduction of human immunoglobulin (Ig) loci into mice in which the endogenous Ig genes have been inactivated is one means of producing fully human monoclonal antibodies. The transgenic mice are immunized with antigen, and conventional hybridoma technology may be used to prepare human monoclonal antibodies with the desired specificity. Fully human antibodies can also be derived from phage-display libraries (as disclosed in Hoogenboom et al., (1991) J. Mol. Biol. 227:381; and Marks et al., (1991) J. Mol. Biol. 222:581).

Nanobodies devoid of light chains based on camelid antibodies are described in, e.g., Chem Biol (2006) 13:1243-4; Coppieters et al., Arthritis Rheum (2006) 54:1856-66.

The term “isolated” refers to a compound, which can be e.g. an antibody or antibody fragment, that is substantially free of other antibodies or antibody fragments having different antigenic specificities. Moreover, an isolated antibody or antibody fragment may be substantially free of other cellular material and/or chemicals. Thus, in some aspects, antibodies provided are isolated antibodies which have been separated from antibodies with a different specificity. An isolated antibody may be a monoclonal antibody. An isolated antibody may be a recombinant monoclonal antibody. The isolated antibody may be substantially pure, or at least 80%, 85%, 90%, 95%, or 99% pure.

An isolated antibody that “specifically binds” to an epitope, isoform or variant of a target has its highest affinity or avidity for the target, but may have some lesser cross-reactivity to other related antigens, e.g., from other species (e.g., species homologs). It is thus capable of recognizing the target antigen in the presence of a heterogeneous population of molecules.

The term “K_{D}”, as used herein, refers to the dissociation constant, which is the ratio of K_{d} to K_{d}. The smaller the number of K_{D}, the higher the affinity. K_{D} can be determined using methods well known in the art, including enzyme-linked immunosorbent assays (ELISA), surface surface plasmon resonance using a biosensor system such as a BIACORE system, or a kinetic exclusion assay such as KINEXA.

Antibodies of the present invention, including pharmaceutical compositions and antibodies for use in the methods described herein, typically have a K_{D} of less than 10⁻⁴M, 10⁻⁵M, 10⁻⁶M, 10⁻⁷M, 10⁻⁸M, 10⁻⁹M, 10⁻¹⁰ M, 10⁻¹¹ M, 10⁻¹²M, 10⁻¹³M, 10⁻¹⁴ M, 10⁻¹⁵ M, or lower.

“Cross competes” means the ability of an antibody or antibody fragment to interfere with the binding of a reference antibody or antibody fragment to a specific antigen, in a competitive binding assay. Cross-competition is present if the antibody or antibody fragment specifically reduces the binding of the reference antibody or antibody fragment by 60% or more, by 70% or more or by 80% or more.

Provided herein are antibodies or antibody fragments that bind to the same epitope as, or that cross-compete with, any of the antibodies disclosed herein. Such antibodies may be determined by methods well known in the art, including enzyme-linked immunosorbent assays (ELISA), surface plasmon resonance using a biosensor system such as a BIACORE system, or a kinetic exclusion assay such as KINEXA. For example, mAbs 5-4A9, 5-2D7, 5-9B12, 5-11D1, 5-14H5 and 5-14A8 all compete with each other for binding to VpreB on NALM-6 cells, indicating that they all bind to a single epitope or to an overlapping linear or conformational epitope. Epitopes may be determined, for example, by deuterium exchange to identify portions of antigen that are contacted (protected) by the antibody), or by creating linear peptide epitopes and determining binding.

Nucleic acid encoding any of the antibodies or antibody fragments disclosed herein can be cloned and expressed using a suitable vector and host cell. Nucleic acid encoding antibody may include nucleotide sequence encoding leader sequences and/or fusion partner sequences.

A vector may comprise a nucleic acid encoding one or more domains, regions or chains of an antibody or antibody fragment, wherein said nucleic acid is operably linked to an expression control sequence. The term “expression vector” or “expression construct” refers to a vector that is suitable for transformation of a host cell in order to express a desired protein (e.g. antibody or antibody fragment), and which contains a heterologous expression control sequence, preferably a heterologous promoter, operably linked to a nucleotide sequence coding for the desired protein.

The term “expression control sequence” refers to a nucleotide sequence that can regulate the transcription, translation, expression or processing of coding sequences to which it is linked. Example expression control sequences include promoters, recognition sites for transcription factors, transcription enhancer sequences, transcription termination sequence, introns, splicing signals, and polyadenylation signals.

The term “host cell” includes progeny of the parent cell, whether or not the progeny is identical in morphology or in genetic make-up to the original parent cell, so long as the gene or vector of interest is present.

Expression of antibodies from host cells may be accomplished with standard techniques. For example, expression vector(s) comprising nucleic acid sequence(s) encoding the heavy and light chains, operably linked to a heterologous expression control sequence, are introduced into a host cell. The host cells are cultured in culture medium for a period of time sufficient to allow for expression of the antibody in the host cells or secretion of the antibody into the culture medium. Antibodies can be recovered from the host cells or culture medium using standard protein purification methods. Suitable mammalian host cells for expressing the recombinant antibodies of the invention include Chinese Hamster Ovary (CHO cells) (including dhfr-CHO cells, described in Urlaub and Chasin, (1980) Proc. Natl. Acad. Sci. USA 77:4216-4220), NSO myeloma cells, COS cells, HEK293 cells, and SP2 cells.

Host cells can also be used to produce functional antibody fragments, such as Fab fragments or scFv molecules. For example, a vector encoding functional fragments of either the light chain and/or the heavy chain of an antibody is introduced into a host cell. Standard molecular biology techniques are used to prepare the recombinant expression vector(s), transfect the host cells, select for transformants, culture the host cells and recover the antibody from the culture medium.

Variants of antibodies are contemplated herein, including antibodies or antibody fragments comprising a VH comprising HC CDR1, CDR2 and CDR3 sequences having at least 60%, 65%, 70%, 75%, 80%, 85%, 90%, 95%, 96%, 97%, 98%, 99% or 100% identity to any of the respective HC CDRs set forth herein; and a VL comprising LC CDR1, CDR2 and CDR3 sequences having at least 60%, 65%, 70%, 75%, 80%, 85%, 90%, 95%, 96%, 97%, 98%, 99% or 100% identity to any of the respective LC CDRs set forth herein. Variations within the CDRs include mutations (insertions, deletions, substitutions) of 1, 2, or 3 amino acids among any of the six CDRs of an antibody, preferably conservative substitutions. For example, there may be 1 or 2 mutations within any of HC CDR1, HC CDR2, HC CDR3, LC CDR1, LC CDR2, or LC CDR3. As one example, one or more CDR amino acids is substituted with histidine to create a pH-responsive antibody. This enhances degradation of the antigen and recycling of the antibody. For example, 1, 2, 3, 4, 5 or 6 amino acids among the six CDRs of an antibody are substituted with histidine. Other examples include reducing asparagine deamidation or aspartate isomerization by substitution with another amino acid. In some embodiments, the total number of CDR mutations within the antibody is minimized, e.g. no more than 6, 5, 4, 3, 2 or 1 mutations total for all six CDRs.

The term “percent identity” or “homology” means the percent of identical residues between the amino acids or nucleotides in the compared molecules and is calculated based on the size of the smallest of the molecules being compared. For these calculations, gaps in alignments (if any) are addressed by a particular mathematical model or computer algorithm. Alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; Pearson and Lipman, Proc. Natl. Acad. Sci. U.S.A. 85:2444, 1988; Altschul et al., Nature Genet. 6:119, 1994. The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet.

Modifications to the antibody constant region may increase or decrease effector function, including ADCC and/or complement dependent cytotoxicity (CDC) activity; decrease antibody-antigen aggregate formation; or reduce the formation of half-antibodies. Reduced fucosylation of the constant region, e.g. at Asn297, can increase ADCC (Shields et al., J. Biol. Chem. 277(30) 2002: 26733-26740).

Antibodies and antibody fragments may also comprise heterologous moieties. Antibody and antibody fragments may be linked, directly or indirectly, to detectable markers, including fluorophores, chemiluminescent agents, enzymatic linkages, radioactive isotopes and heavy metals or magnetic agents or paramagnetic particles such as superparamagnetic iron oxide.

Antibody Drug Conjugates

Antibody-drug conjugates comprise an antibody (or antigen-binding fragment of an antibody) and a drug, such as a cytotoxic agent or toxin.

Monoclonal antibody therapy has been established for the targeted treatment of patients with cancer, inflammatory, immunological, and angiogenic disorders. For cancer, one effective approach for enhancing the anti-tumor potency of antibodies involves linking cytotoxic drugs or toxins to monoclonal antibodies that are capable of being internalized by a target cell. These linked complexes are termed antibody-drug conjugates (ADC). Upon administration to a patient, ADCs bind to target cells via their antibody component and become internalized, allowing the toxin or cytotoxic drug to exert its effect (see, e.g., U.S. Patent Appl. Publ. Nos. US2005/0180972 and US2005/0123536). Table 1 lists exemplary ADCs currently in clinical development or FDA-approved, along with representative citations.

TABLE 1
Representative Clinical or Commercial Stage Antibody-drug Conjugates

| ADC | Target | Citation |
|---|---|---|
| enapotamab vedotin | AXL | WO2017009258A1 |
| belantamab mafodotin | BCMA | Trudel et al., Blood Cancer J. 9: 37, 2019 |
| XmAb5574 | CD19 | Woyach et al., Blood 124: 3553, 2014 |
| MT-3724 | CD20 | Huang et al., Blood Cancer J. 8: 33, 2018 |
| moxetumomab | CD22 | Dhillon, Drugs 78: 1763, 2018 |
| pasudotox | | |
| camidanlumab tesirine | CD25 | Flynn et al., Mol. Cancer. Ther. 15: 2709, 2016 |
| gemtuzumab ozogamicin | CD33 | Godwin et al., Leukemia 31: 1855, 2017 |
| AGS67E | CD37 | Pereira et al., Mol. Cancer Ther. 14: 1650, 2015 |
| polatuzumab vedotin | CD79b | Tilly et al., Lancet Oncol. 20: 998, 2019 |
| IMGN632 | CD123 | Angelova et al., Haematologica. 104: 749, 2019 |
| indatuximab ravtansine | CD138 | Jagannath et al., Clin. Lymphoma |
| | | Myeloma Leuk. 19: 372, 2019 |
| MEN1309 | CD205 | Merlino et al., Mol. Cancer Ther. Jun 21, 2019 |
| SAR408701 | CEACAM5 | Bouillon-Pichault et al., J. Clin. |
| | | Pharmacol. 57: 865, 2017 |
| rovalpituzumab tesirine | DLL3 | Lashari et al., Drugs R D. 18: 255, 2018 |
| depatuxizumab | EGFR | Lassman et al., Neuro. Oncol. 21: 106, 2019 |
| mafodotin | | |
| AGS62P1 | FLT3 | Snyder et al., Mol. Pharm. 15: 2384, 2018 |
| mirvetuximab | FOLR1 | Moore et al., Future Oncol. 14: 1669, 2018 |
| soravtansine | | |
| T-DM1 | HER2 | Okines, Rev Recent Clin Trials. 12: 216, 2017 |
| U3-1402 | HER3 | Yonesaka et al., Oncogene 38: 1398, 2019 |
| ladiratuzumab vedotin | LIV-1 | Sussman et al., Mol. Cancer Ther. 13: 2991, 2014 |
| anetumab ravtansine | mesothelin | Quanz et al., Oncotarget 9: 34103, 2018 |
| telisotuzumab vedotin | MET | Strickler et al., J. Clin. Oncol. 36: 3298, 2018 |
| enfortumab vedotin | Nectin-4 | Challita-Eid et al., Cancer Res. 76: 3003, 2016 |
| PF-06647020 | PTK7 | Damelin et al., Sci. Transl. Med. 9: eaag2611, 2017 |
| tisotumab vedotin | TF | De Bono et al., Lancet Oncol. 20: 383, 2019 |
| sacituzumab govitecan | TROP2 | Gray et al., Clin. Cancer Res. 23: 5711, 2017 |

As with a mature BCR, cross-linking of the pre-BCR results in its internalization. This can be accomplished experimentally with an IgM-specific antibody (Salamero et al., Eur. J. Immunol. 25:2757, 1995). Alternatively, auto-crosslinking of the pre-BCR via the unique region of lambda-5 component of the SLC results in internalization and attenuation of pre-BCR signaling (Ohnishi and Melchers, Nat. Immunol. 4:849, 2003; Knoll et al., J. Immunol. 188:6010, 2012). These observations suggest that a VpreB or lambda-5 antibody may cross-link the pre-BCR and provoke its internalization, enabling its use as a vehicle to deliver an ADC to the interior of the cancer cell.

Drugs may be linked to the antibody or antibody fragment directly or indirectly, and reversibly or irreversibly. Antibodies may be linked to nanoparticles, including nanospheres, nanocapsules, liposomes, dendrimers, polymeric micelles, niosomes, and polymeric nanoparticles (Fay and Scott, Immunotherapy 3(3):381-394, 2011).

A variety of dipeptide-based cleavable linkers useful for linking drugs to antibodies have been described. See Dubowchik et al., 1998, J. Org. Chem. 67:1866-1872; Dubowchik et al., 1998, Bioorg. Med. Chem. Lett. 8:3341-3346; Walker et al., 2002, Bioorg. Med. Chem. Lett. 12:217-219; Walker et al., 2004, Bioorg. Med. Chem. Lett. 14:4323-4327; and Francisco et al., 2003, Blood 102:1458-1465. Dipeptide linkers include Val-Cit and Phe-Lys. Other linkers include the bifunctional para-aminobenzyl alcohol group, which is linked to the peptide through the amino group, forming an amide bond, while amine containing drugs may be attached through carbamate functionalities to the benzylic hydroxyl group of the linker (to give a p-amidobenzylcarbamate, PABC). The resulting prodrugs are activated upon protease-mediated cleavage. A variety of cleavable-glucuronic acid-based linkers useful for linking drugs such as auristatins, camptothecin and doxorubicin analogues, CBI minor-groove binders, and psymberin to antibodies have been described (see, Jeffrey et al., 2006, Bioconjug. Chem. 17:831-840; Jeffrey et al., 2007, Bioorg. Med. Chem. Lett. 17:2278-2280; and Jiang et al., 2005, J. Am. Chem. Soc. 127:11254-11255. Cleavable linkers may also include a disulfide group. Disulfides are thermodynamically stable at physiological pH and are designed to release the drug upon internalization inside cells, wherein the cytosol provides a significantly more reducing environment compared to the extracellular environment. Other hydrolytically degradable linkages include, but are not limited to, carbonate linkages; imine linkages resulting from reaction of an amine and an aldehyde; phosphate ester linkages formed by reacting an alcohol with a phosphate group; acetal linkages that are the reaction product of an aldehyde and an alcohol; orthoester linkages that are the reaction product of a formate and an alcohol.

The antibody may be linked to a drug such as a cross-linking agent, an anti-microtubule agent and/or anti-mitotic agent, or any cytotoxic agent suitable for mediating killing of tumor cells. Exemplary cytotoxic agents include, but are not limited to, doxorubicin, mitomycin, camptothecin, tallysomycin and auristatin or auristatin family members, a pyrrolobenzodiazepine (PDB), anthramycin, a maytansinoid, dolastatin, calicheamicin, nemorubicin and its derivatives, PNU-159682, anthracycline, vinca alkaloid, taxane, trichothecene, CC1065, camptothecin, elinafide, a combretastain, a dolastatin, a duocarmycin, an enediyne, a geldanamycin, an indolino-benzodiazepine dimer, a puromycin, a tubulysin, a hemiasterlin, a spliceostatin, or a pladienolide, as well as stereoisomers, isosteres, analogs, and derivatives thereof that have cytotoxic activity. Exemplary dolastatins and auristatins include, but are not limited to, dolastatin 10, auristatin E, auristatin F, auristatin EB (AEB), auristatin EFP (AEFP), MMAD (Monomethyl Auristatin D or monomethyl dolastatin 10), MMAF (Monomethyl Auristatin F or N-methylvaline-valine-dolaisoleuine-dolaproine-phenylalanine), MMAE (Monomethyl Auristatin E or N-methylvaline-valine-dolaisoleuine-dolaproine-norephedrine), 5-benzoylvaleric acid-AE ester (AEVB), and other auristatins (see, for example, U.S. Publication No. 2013/0129753). The calicheamicin family of antibiotics, and analogues thereof, are capable of producing double-stranded DNA breaks at sub-picomolar concentrations (Hinman et al., Cancer Res 53:3336-3342, 1993; Lode et al., Cancer Res 58:2925-2928, 1998). Exemplary anthracyclines include doxorubicin, epirubicin, idarubicin, daunomycin, daunorubicin, doxorubicin, epirubicin, nemorubicin, valrubicin and mitoxantrone, and derivatives thereof. For example, PNU-159682 is a potent metabolite (or derivative) of nemorubicin (Quintieri et al., Clin Cancer Res 11(4):1608-1617, 2005). Nemorubicin is a semisynthetic analog of doxorubicin with a 2-methoxymorpholino group on the glycoside amino of doxorubicin (Grandi et al., Cancer Treat Rev 17:133, 1990; Ripamonti et al., Br J Cancer 65:703-707, 1992).

Bispecific or Multispecific Antibodies

Multi-specific antibodies are recombinant proteins or immunoglobulins comprised of antigen-binding fragments of two or more different monoclonal antibodies. For example, bispecific antibodies are comprised of antigen-binding fragments of two different monoclonal antibodies. Thus, bispecific antibodies bind two different antigens and trispecific antibodies bind three different antigens. Multi-specific antibodies can be used for cancer immunotherapy by simultaneously targeting, for example, both CTLs (such as a CTL receptor component such as CD3) or effector natural killer (NK) cells, and at least one tumor antigen. Bispecific antibodies may be heterodimeric, comprising a second different types of VH region and optionally a second different type of VL region, and thus bind two different antigens. Stability of heterodimeric pairing can be improved through means known in the art, e.g. through knobs-in-holes mutations. Ridgway et al., Protein Eng (1996) 9:617-21; Zhu et al. (1997) Protein Sci 6:781-788. Alternatively, different types of VH regions (and/or VL regions) can be chemically crosslinked.

Bispecific or multispecific antibodies include bivalent bispecific T cell engagers (BITE) or tetravalent bispecific antibodies (TandAb). Several bispecific antibody formats have been developed. The BiTE (bispecific T cell engager) molecules have been very well characterized (reviewed in Nagorsen and Bauerle, Exp Cell Res 317, 1255-1260 (2011)). BiTEs are tandem scFv molecules wherein two scFv molecules are fused by a flexible linker. Further bispecific formats being evaluated for T cell engagement include diabodies (Holliger et al., Prot Eng 9, 299-305 (1996)) and derivatives thereof, such as tandem diabodies (Kipriyanov et al., J Mol Biol 293, 41-66 (1999)). A more recent development are the so-called DART (dual affinity retargeting) molecules, which are based on the diabody format but feature a C-terminal disulfide bridge for additional stabilization (Moore et al., Blood 117, 4542-51 (2011)).

Therapeutic antibodies may be engineered to bind two distinct antigens, such as a tumor cell target and CD3 on effector T cells to bring the two cell types into close proximity and provoke activation of the T cell and destruction of the tumor cell. Such antibodies, termed bispecifics, have been shown to inhibit B cell and B cell cancer proliferation and to promote cell death by targeting several cell surface receptors, including CD21, CD81, and CD19 (Hatterer et al., MAbs 11:322, 2019). Recent clinical results with CD19/CD3 bispecific antibodies attest to the therapeutic potential of this approach across multiple B cell malignancies (Hammer 0, MAbs 4:571, 2012). These data suggest that a VpreB or lambda-5 bispecific antibody may also be used as a therapeutic option when designed to minimize internalization. Table 2 lists exemplary bispecific therapies currently in clinical development or FDA-approved, along with representative citations.

TABLE 2
Representative Clinical or Commercial Stage Bispecific Antibodies

| Antibody | Targets | Citation |
|---|---|---|
| faricimab | ANG2 X VEGF | Sahni et al., Ophthalmol. Mar 21. 2019 |
| MGD009 | B7H3 X CD3 | US9441049B2 |
| REGN5458 | BCMA X CD3 | Dilillo et al., Blood 132: 1944, 2018 |
| blinatumomab | CD19 X CD3 | Wilke and Gökbuget, Expert Opin. |
| | | Drug Saf. 16: 1191, 2019 |
| REGN1979 | CD20 X CD3 | Smith et al., Sci Rep. 5: 17943, 2015 |
| AFM13 | CD30 X CD16A | Rothe et al., Blood. 125: 4024, 2015 |
| AMG 330 | CD33 X CD3 | Friedrich et al., Mol. Cancer Ther. 13: 1549, 2014 |
| JNJ-63709178 | CD123 X CD3 | Gaudet et al., Blood 128: 2824, 2016 |
| CEA-TCB | CEA X CD3 | Bacac et al., Clin. Cancer Res. 22: 3286, 2016 |
| MCLA-117 | CLEC12A X CD3 | Van Loo et al., Expert Opin. Biol. Ther. 19: 721, 2019 |
| OMP-305B83 | DLL4 X VEGF | Jimeno et al., Invest. New Drugs. 37(3): 461, 2019 |
| AMG 596 | EGFRvIII X CD3 | Rosenthal et al., J. Clin. Oncol. 37(15): suppl., 2019 |
| emicizumab | Factor IXa X | Rodriguez-Merchan and Valentino, |
| | Factor X | Haemophil. 25: 11, 2019 |
| MGD007 | gpA33 X CD3 | Moore et al., Mol. Cancer Ther. 17: 1761, 2018 |
| ZW25 | Her2 X Her2 | Cancer Discov. 9: 8, 2019 |
| GBR 1302 | Her2 X CD3 | Wermke et al., J. Clin. Oncol. 35(1): suppl |
| MCLA-128 | Her2 X Her3 | De Vries Schultink et al., Invest. |
| | | New Drugs. 36: 1006, 2018 |
| FS118 | LAG3 X PD-L1 | Kraman et al., Cancer Res. Proceedings, |
| | | Abstr. 2719, 2018 |
| JNJ-61186372 | MET X EGFR | Moores et al., Cancer Res. 76: 3942, 2016 |
| ES414 | PSMA X CD3 | Hernandez-Hoyos et al., Mol. |
| | | Cancer Ther. 15: 2155, 2016 |
| XmAb18087 | SSTR2 X CD3 | Lee et al., Cancer Res. Proceedings, Abstr. 3633, 2017 |

### Effector T Cells

T cells may be engineered to express recombinant receptors that bind to VpreB or lambda-5 that comprise one or more of the CDRs described herein, preferably all six CDRs of each of the antibodies or disclosed herein. For example, a T cell may express a chimeric antigen receptor (CAR) that includes an antigen-binding portion (such as a single domain antibody or scFv) and a signaling domain, such as a signaling domain from a T cell receptor (e.g. CD3zeta). Typically, CARs are comprised of an antigen-binding moiety, optionally an extracellular hinge and spacer element, a transmembrane domain and an endodomain that performs signaling functions. The spacer/hinge region typically includes sequences from IgG subclasses, such as IgG1, IgG4, IgD and CD8 domains. The transmembrane domain can be derived from a variety of different T cell proteins, such as CD3zeta, CD4, CD8 or CD28. Several different endodomains have been used to generate CARs. The endodomain typically includes a signaling chain having an immunoreceptor tyrosine-based activation motif (ITAM), such as CD3zeta or Fc-εRIγ. In some instances, the endodomain further includes the intracellular portion of at least one additional co-stimulatory domain, such as CD28, 4-1BB (CD137), ICOS, OX40 (CD134), CD27 and/or DAP10.

T cells can also be engineered to replace the natural T cell receptor (TCR) hypervariable domains with one or more of the CDRs described herein, (e.g. HC CDR1, CDR2, and CDR3 and/or LC CDR1, CDR2 and CDR3), and thereby alter the antigen-specificity of the TCR (Sharpe et al., Dis Model Mech. 2015 April; 8(4): 337-350).

In addition to monoclonal antibodies, ADCs, and bispecific constructs, CAR-T cells represent another strategy to exploit an antibody's specificity to introduce an effector T cell to a tumor cell. CAR-T cells are genetically engineered to express a chimeric receptor with intracellular domains that initiate an activating cascade when the extracellular, antibody V region binds its cognate antigen. This strategy has demonstrated striking success in controlling hematologic malignancies, but toxicity remains an issue due to both on-target/off-tumor interactions and systemic toxicity associated with massive immune activation and cytokine release (Martinez and Moon, Front. Immunol. 10:1, 2019). Given the restricted expression of VpreB and lambda-5 in B cell development, the pre-BCR is a unique target with limited potential for inducing CAR-T-associated toxicity (Wilson et al., WO216127043 A1). Table 3 lists exemplary CAR-T therapies currently in clinical development or FDA-approved, along with representative citations.

TABLE 3
Representative Clinical or Commercial Stage CAR-T Cell Therapies

| CAR-T | Target | Citation |
|---|---|---|
| bb2121 | BCMA | Raje et al., N. Engl. J. Med. 380: 1726, 2019 |
| axicabtagene | CD19 | Roberts et al., Leuk. Lymphoma. 59: 1785, 2018 |
| ciloleucel | | |
| tisagenlecleucel- | CD19 | Thomas and Paubelle, Expert Opin. Biol. Ther. |
| T | | 18: 1095, 2018 |
| UCART123 | CD123 | Cai et al., Cancer Res. Abstr. 2560, 2018 |
| JCAR023 | CD171 | Kunkele et al., Clin. Cancer Res. 23: 466, 2017 |
| CART- | EGFRvIII | O’Rourke et al., Sci. Transl. Med. 9: eaaa0984, 2017 |
| EGFRvIII | | |
| MB-101 | IL13Rα2 | Brown et al., N. Engl. J. Med. 375: 2561, 2016 |
| CART-meso | mesothelin | Beatty et al., Gastroenterol. 155: 29, 2018 |
| JCAR020 | MUC-16 | Koneru et al., J. Transl. Med. 13: 102, 2015 |
| CYAD-01 | NKG2D | Lonez et al., Curr. Res. Transl. Med. 66: 53, 2018 |
| BPX-601 | PCSA | Becerra et al., J. Clin. Oncol. 37(15)suppl: 2536, 2019 |
| JCAR024 | ROR1 | Berger et al., Cancer Immunol. Res. 3: 206, 2015 |

### Methods of Treatment

Compositions of the present disclosure may be used for therapeutic or prophylactic treatment of subjects. “Subject” includes mammals and humans. The terms “human” and “subject” are used interchangeably herein. Similarly, “patient” is also a subject.

An “effective amount” refers to the amount of a compound that, when administered to a subject for treating a disease, or at least one of the clinical symptoms of a disease or disorder, is sufficient to affect such treatment for the disease, disorder, or symptom. Generally, an effective amount is sufficient to reduce the severity and/or frequency of signs or symptoms, or eliminate the signs or symptoms, or prevent the occurrence of symptoms and/or improve or remediate the damage resulting from the disease. An “effective amount” is a therapeutically effective amount or a prophylactically effective amount. As those skilled in the art will recognize. this amount is typically not limited to a single dose but may comprise multiple dosages over a significant period of time as required to bring about a therapeutic or prophylactic response in the subject. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation. A typical dosage may range from about 0.1 mg/kg to up to about 100 mg/kg or more, depending on the factors mentioned above. In other embodiments, the dosage may range from 1 mg/kg up to about 100 mg/kg or 5 mg/kg up to about 100 mg/kg.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, or inhibiting at least one physical parameter which may not be discernible to the subject. Further, “treating” or “treatment” refers to delaying the onset of the disease or disorder or at least symptoms thereof in a subject which may be exposed to or predisposed to a disease or disorder even though that subject does not yet experience or display symptoms of the disease or disorder.

In various embodiments, single or multiple administrations of the pharmaceutical compositions are administered depending on the dosage and frequency as required and tolerated by the subject. In any event, the composition should provide a sufficient quantity of at least one of the compounds disclosed herein to effectively treat the subject. The dosage can be administered once but may be applied periodically until either a therapeutic result is achieved or until side effects warrant discontinuation of therapy.

Pharmaceutical compositions can be delivered by routes including intravenous, intramuscular, intraperitoneal, oral (including sublingual), intranasal, aerosol (for intrapulmonary administration), parenteral, subcutaneous, transdermal, mucosal, topical, intra-tumoral, and also ex vivo, for example, by treating tumors or dendritic cells to express the antibody or antibody fragment and then reintroducing them into the patient.

The dosing frequency of the administration of the pharmaceutical composition depends on the nature of the therapy and the particular disease being treated. Treatment of a subject with a therapeutically effective amount of a compound, of the invention can include a single treatment or, preferably, can include a series of treatments. In a preferred example, a subject is treated with compound daily, one time per week or biweekly.

Cancers that may be treated with the antibodies, antibody fragments, bispecific antibodies, ADCs, or T cells described herein include, but are not limited to include, bladder cancer, brain cancer, breast cancer, bone marrow cancer, cervical cancer, chronic lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small cell lung cancer, chronic lymphocytic leukemia, myeloma, prostate cancer, or spleen cancer.

The methods of the invention are suitable for treating hematologic malignancies, such as, BCP-ALL, lymphomas, thymomas, T cell acute lymphoblastic leukemia (T-ALL), and acute myelogenous leukemia (AML), diffuse large B cell lymphoma, follicular lymphoma, marginal zone B cell lymphoma, T cell lymphoma, Non-Hodgkin's lymphoma (NHL). B cell NHLs include Burkitt lymphoma, chronic lymphocytic leukemia/small lymphocytic lymphoma, diffuse large B-cell lymphoma, follicular lymphoma, immunoblastic large cell lymphoma, precursor B-lymphoblastic lymphoma, and mantle cell lymphoma.

Preferred diseases for treatment according to the methods described herein include acute lymphoblastic leukemia (ALL), acute myelogenous leukemia (AML), T-cell acute lymphoblastic leukemia (T-ALL), chronic lymphocytic leukemia (CLL), thymoma, lymphoma, mantel cell lymphoma (MCL), marginal zone lymphoma (MZL), diffuse large B cell lymphoma (DLBCL), follicular lymphoma (FL), Waldenstrom macroglobulinemia (WM), and multiple myeloma (MM).

The methods of treatment are also useful for autoimmune or immune-mediated inflammatory disease, including inflammatory bowel disease, ulcerative colitis, Crohn's disease, multiple sclerosis, psoriasis, rheumatoid arthritis, systemic lupus erythematosus, type 1 diabetes, vasculitis, asthma, eczema and atopic dermatitis, fibrosis, graft rejection, and graft-versus-host-disease.

The methods of treatment include co-administration of a second therapeutic agent, such as other cancer therapeutic agents, or agents that activate the immune response such as checkpoint inhibitors, co-activating receptor agonists, and cancer or pathogen-focused vaccines. When administered as a combination, the therapeutic agents can be formulated as separate compositions that are administered at the same time or sequentially at different times, or the therapeutic agents can be given as a single composition. The phrase “co-therapy” (or “combination-therapy”) is intended to embrace administration of each agent in a sequential manner in a regimen that will provide beneficial effects of the drug combination, and is intended as well to embrace co-administration of these agents in a substantially simultaneous manner.

Additional cancer therapeutic agents include antineoplastic agents, anti-angiogenic agents, chemotherapeutic agents and peptidyl cancer therapy agents, in yet another embodiment, the antineoplastic agents are selected from antibiotic-type agents, alkylating agents, antimetabolite agents, hormonal agents, immunological agents, interferon-type agents, kinase inhibitors, miscellaneous agents and combinations thereof. It is noted that the additional therapeutic agents may be traditional small organic chemical molecules or can he be macromolecules such as a proteins, antibodies, peptibodies, DNA, RNA or fragments of such macromolecules.

Examples of specific therapeutic agents that can be used in combination with one or more antibodies or antibody fragments of the present invention include: atezolizumab, pembrolizumab, ipilimumab, methotrexate, tamoxifen, fluorouracil, 5-fluorouracil, hydroxyurea, mercaptopurine, cisplatin, carboplatin, daunorubicin, doxorubicin, etoposide, vinblastine, vincristine, paclitaxel, thioguanine, idarubicin, dactinomycin, imatinib, gemcitabine, altretamine, asparaginase, bleomycin, capecitabine, carmustine, cyclophosphamide, cytarabine, docetaxel, idarubicin, ifosfamide, irinotecan, fludarabine, mitosmycin, mitoxantrone, topotecan, vinorelbine, adriamycin, mithramycin, imiquimod, alemtuzmab, exemestane, bevacizumab, cetuximab, azacitidine, clofarabine, decitabine, desatinib, dexrazoxane, docetaxel, epirubicin, oxaliplatin, erlotinib, raloxifene, fulvestrant, letrozole, gefitinib, gemtuzumab, trastuzumab, gefitinib, ixabepilone, lapatinib, lenalidomide, aminolevulinic acid, temozolomide, nelarabine, sorafenib, nilotinib, pegaspargase, pemetrexed, rituximab, dasatinib, thalidomide, bexarotene, temsirolimus, bortezomib, vorinostat, capecitabine, zoledronic acid, anastrozole, sunitinib, aprepitant and nelarabine, or pharmaceutically acceptable salts thereof.

The methods of treatment include concurrent administration of surgery, radiation, or conventional chemotherapy. Radiosensitizers are known to increase the sensitivity of cancerous cells to the toxic effects of electromagnetic radiation.

The methods of treatment also include co-administration of a checkpoint inhibitor, for example, a PD-1 antagonist (e.g., anti-PD-1 antibody or anti-PD-L1 antibody), a CTLA-4 antagonist (e.g. anti-CTLA-4 antibody), LAG-3 antagonist, a CD80 antagonist, a CD86 antagonist, a Tim-3 antagonist, a TIGIT antagonist, a CD20 antagonist, a CD96 antagonist, an IDOl antagonist, a STING antagonist, a GARP antagonist, a CD40 antagonist, an A2aR antagonist, a CEACAM1 (CD66a) antagonist, a CEA antagonist, a CD47 antagonist, a PVRIG antagonist, a TDO antagonist, a VISTA antagonist, or a KIR antagonist. Antagonists include antibodies and small molecule inhibitors.

### Pharmaceutical Compositions

Pharmaceutical compositions of the present disclosure may comprise any of the antibodies or antibody fragments described herein and a pharmaceutically suitable carrier, excipient or diluent.

In some embodiments, the pharmaceutical composition comprises any of the antibodies or antibody fragments disclosed herein at a purity level suitable for administration to a patient. In some embodiments, the analog has a purity level of at least about 90%, preferably above about 95%, more preferably above about 99%, and a pharmaceutically acceptable diluent, carrier or excipient.

The pharmaceutical compositions may be formulated to achieve a physiologically compatible pH. For example, buffers are used to maintain the composition at physiological pH or at slightly lower pH, typically within a pH range of from about 5 to about 8, e.g. about 6 to about 8 or about 7.

The primary vehicle or carrier in a pharmaceutical composition may be either aqueous or non-aqueous in nature. Additional excipients may be included, including buffers (e.g., phosphate, acetate, and histidine), tonicity agents/stabilizers (sugars such as sucrose, polyols such as sorbitol), bulking agents (lyoprotectants such as mannitol), surfactants (e.g., polysorbates or poloxamers), antioxidants (e.g., methionine), optionally metal ions/chelating agents (e.g., ethylenediaminetetraacetic acid, EDTA), or preservatives (e.g., benzyl alcohol, benzylalkonium).

For example, a suitable vehicle or carrier may be water for injection, physiological saline solution, Hanks' solution, Ringer's solution, or physiologically buffered saline. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5. The composition may further comprise disaccharide sugars (e.g., sucrose, trehalose, maltose, and lactose) or polyols (e.g., mannitol, sorbitol, and glycerol).

Aqueous injection suspensions may contain substances that increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

When parenteral administration is contemplated, the therapeutic compositions for use in this invention may be in the form of a pyrogen-free, parenterally acceptable aqueous solution comprising pharmaceutically acceptable excipients, preferably isotonic, and optionally comprising preservatives. Alternatively, formulations may include injectable microspheres, bio-erodible particles, polymeric compounds (polylactic acid, polyglycolic acid), beads, or liposomes that provides for the controlled or sustained release of the product. Further, the antibody or antibody fragment may be formulated as a lyophilizate using appropriate lyoprotectants.

### DESCRIPTION OF SEQUENCE LISTING

SEQ ID NO:1 human VpreB sequence (with leader)
SEQ ID NO:2 mouse VpreB1 sequence (with leader)
SEQ ID NO:3 human lambda-5 sequence (with leader)
SEQ ID NO:4 human IgG1 constant region sequence
SEQ ID NO:5 human Ig kappa constant region sequence
SEQ ID NO:6-11: VpreB antibody VH (mouse parental)
SEQ ID NO:6 5-2D7 VH
SEQ ID NO:7 5-4A9 VH
SEQ ID NO:8 5-9B12 VH
SEQ ID NO:9 5-11D1 VH
SEQ ID NO:10 5-14A8 VH
SEQ ID NO:11 5-14H5 VH
SEQ ID NO:12-17: VpreB antibody VL (mouse parental)
SEQ ID NO:12 5-2D7 VL
SEQ ID NO:13 5-4A9 VL
SEQ ID NO:14 5-9B12 VL
SEQ ID NO:15 5-11D1 VL
SEQ ID NO:16 5-14A8 VL
SEQ ID NO:17 5-14H5 VL
SEQ ID NO:18-40: HC framework regions (FRs) and complementarity-determining regions (CDRs) from mouse parental VpreB mAbs
SEQ ID NO:41-57: LC FRs and CDRs from mouse parental VpreB mAbs
SEQ ID NO:58-63: VpreB antibody CDR consensus sequences
SEQ ID NO:64-69: DNA encoding VpreB antibody VH (mouse parental)
SEQ ID NO:70-75: DNA encoding VpreB antibody VL (mouse parental)
SEQ ID NO:76: lambda-5 antibody 4-15E6 VH (mouse parental)
SEQ ID NO:77: lambda-5 antibody 4-15E6 VL (mouse parental)
SEQ ID NO:78-84: HC FRs and CDRs from mouse parental lambda-5 mAbs
SEQ ID NO:85-91: LC FRs and CDRs from mouse parental lambda-5 mAbs
SEQ ID NO:92: DNA encoding lambda-5 antibody 4-15E6 VH (mouse parental)
SEQ ID NO:93: DNA encoding lambda-5 antibody 4-15E6 VL (mouse parental)
SEQ ID NO:94-103: additional lambda-5 antibody VH (mouse parental)
SEQ ID NO:94 4-6D12 VH
SEQ ID NO:95 4-5G11 VH
SEQ ID NO:96 4-7A6 VH
SEQ ID NO:97 4-7C1 VH
SEQ ID NO:98 4-9H8 VH
SEQ ID NO:99 4-12G1 VH
SEQ ID NO:100 4-17G9 VH
SEQ ID NO:101 4-18G6 VH
SEQ ID NO:102 4-19A9 VH
SEQ ID NO:103 4-20D2 VH
SEQ ID NO:104-113: additional lambda-5 antibody VL (mouse parental)
SEQ ID NO:104 4-6D12 VL
SEQ ID NO:105 4-5G11 VL
SEQ ID NO:106 4-7A6 VL
SEQ ID NO:107 4-7C1 VL
SEQ ID NO:108 4-9H8 VL
SEQ ID NO:109 4-12G1 VL
SEQ ID NO:110 4-17G9 VL
SEQ ID NO:111 4-18G6 VL
SEQ ID NO:112 4-19A9 VL
SEQ ID NO:113 4-20D2 VL
SEQ ID NO:114-141: HC FRs and CDRs from additional mouse parental lambda-5 mAbs
SEQ ID NO:142-163: LC FRs and CDRs from additional mouse parental lambda-5 mAbs
SEQ ID NO:164-171: Lambda-5 antibody CDR consensus sequences
SEQ ID NO:172-181: DNA encoding additional lambda-5 antibody VH (mouse parental)
SEQ ID NO:182-191: DNA encoding additional lambda-5 antibody VL (mouse parental)

### EXAMPLES

### Example 1. The Pre-BCR is Expressed by Leukemia and Lymphoma Cells

This example sets forth evidence for SLC component expression in hematologic malignancies in addition to BCP-ALL, such as lymphomas, thymoma, T cell acute lymphoblastic leukemia, and acute myelogenous leukemia.

BCP-ALL arises from B-lymphogenesis arrested at the pre-B cell stage of differentiation. Pre-B cells express a pre-BCR comprising the mu HC and the SLC. It has been reported, however, that the majority of BCP-ALL cells lack a functional pre-BCR (Eswaran et al., Leukemia 29:1623, 2015; Muschen, Blood 125:3688, 2015). Indeed, Geng et al. (Cancer Cell 27:409, 2015) found evidence for tonic pre-BCR signaling in only 13.5% of BCP-ALL cases, although cell surface expression of the receptor was found on 39% of patient-derived BCP-ALL xenograft samples or cell lines. Cytoplasmic staining for VpreB and lambda-5, however, found 80%-90% of primary BCP-ALL cases were pre-BCR positive (Tsuganezawa et al., Blood 92:4317, 1998). This apparent lack of consensus on pre-BCR expression in BCP-ALL likely arises from limited sample sizes and karyotypic variability amongst cases.

Careful review and analysis of expression data indicate that the pre-BCR is more extensively expressed than much of the literature implies, not only in BCP-ALL but also in subsets of other leukemias and lymphomas.

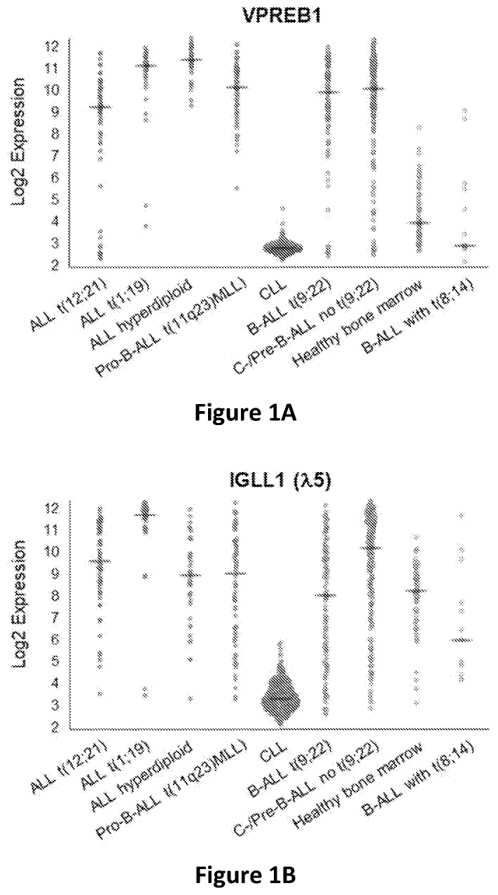

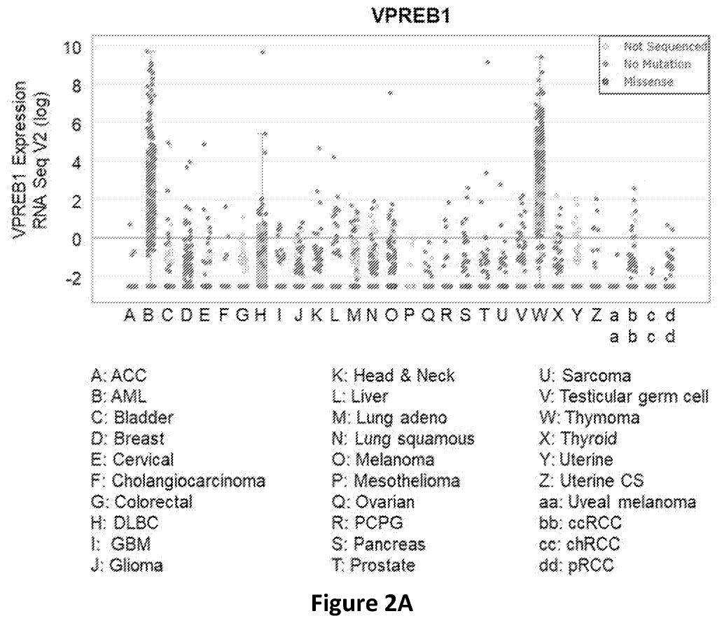

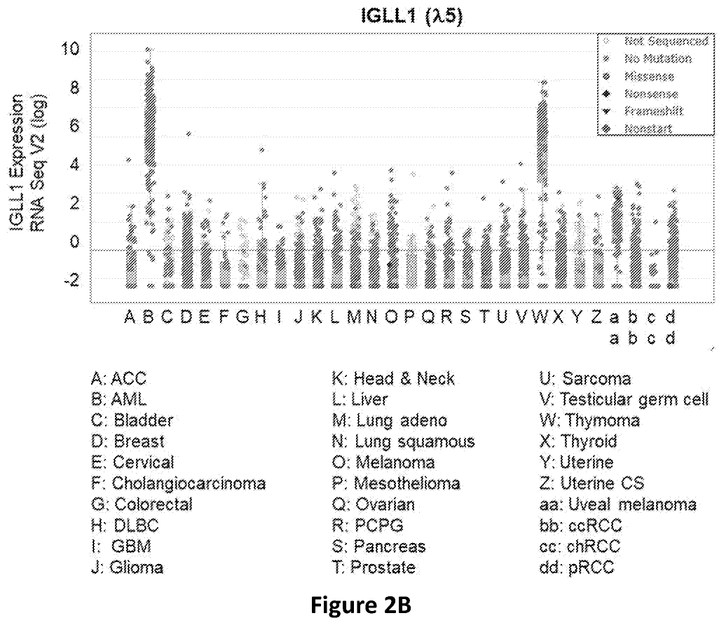

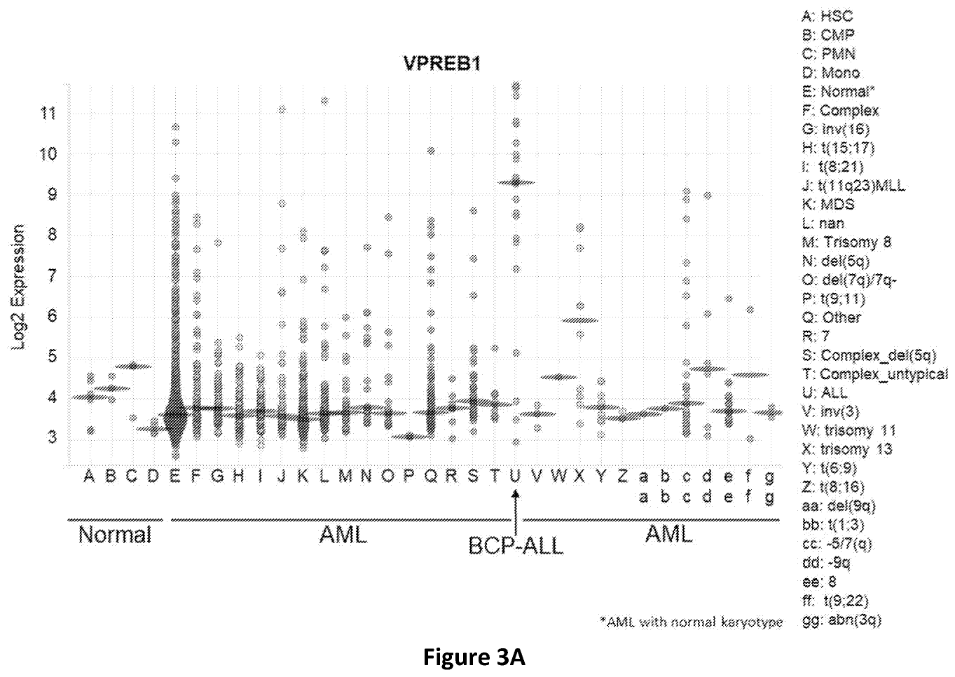

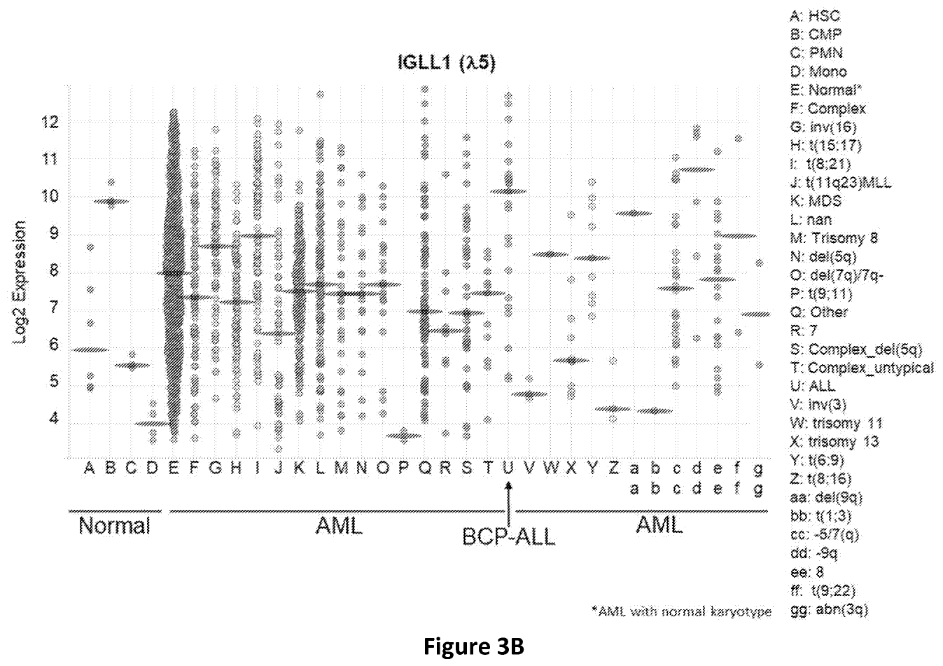

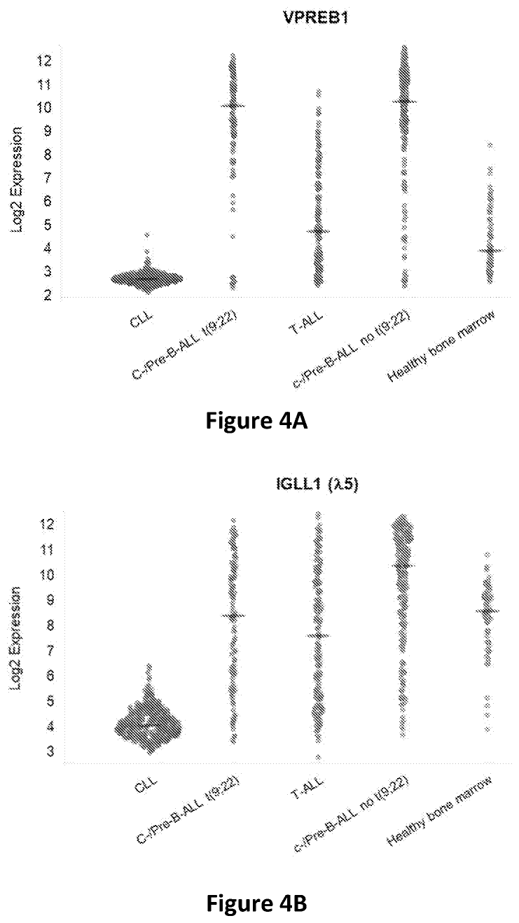

Both VpreB and lambda-5 mRNA are expressed in >90% of BCP-ALL patient samples, suggesting that most patients could benefit from a pre-BCR-targeted therapy (FIGS. 1A and 1B; http://servers.binf.ku.dk/bloodspot/php/help.php; Bagger et al., Nucl. Acids Res. 44:D917, 2016; Haferlach et al., J. Clin. Oncol. 28:2529, 2008). Surprisingly, there is also evidence that the pre-BCR is expressed by a substantial number of AML and thymoma patient samples (FIGS. 2A and 2B; https://www.cbioportal.org/; Gao et al. Sci. Signal. 6:p 11, 2013; Cerami et al. Cancer Discov. 2:401, 2012). Expression of the pre-BCR in a subset of AML patients is further supported by the BloodSpot resource (FIGS. 3A and 3B; http://servers.binf.ku.dk/bloodspot/php/help.php; Bagger et al., Nucl. Acids Res. 44:D917, 2016; Haferlach et al., J. Clin. Oncol. 28:2529, 2008). A similar degree of pre-BCR expression is observed in T-ALL (FIGS. 4A and 4B; http://servers.binf.ku.dk/bloodspot/php/help.php; Bagger et al., Nucl. Acids Res. 44:D917, 2016; Haferlach et al., J. Clin. Oncol. 28:2529, 2008).

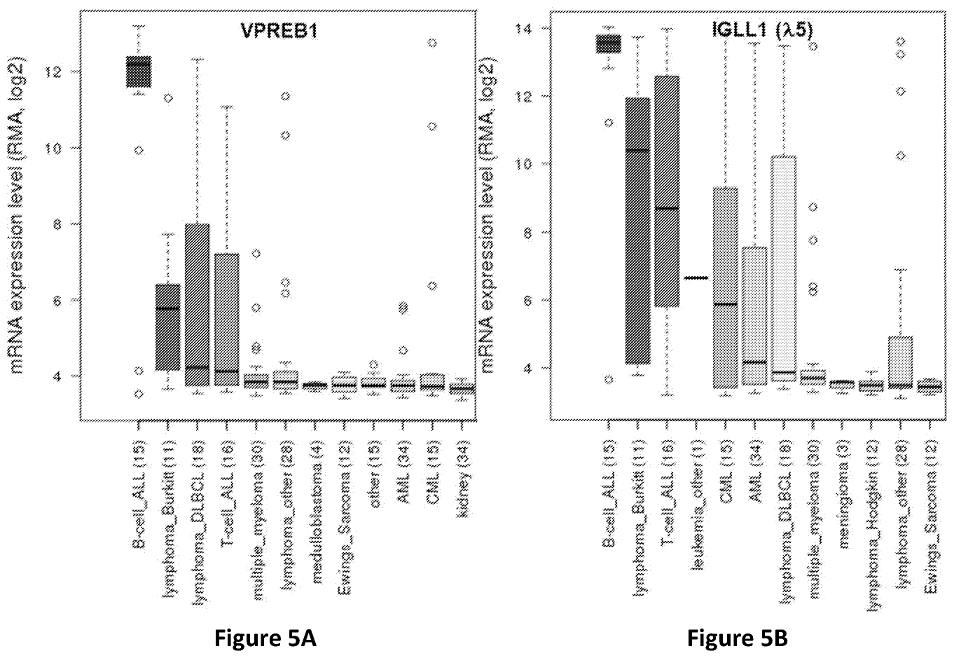

Pre-BCR expression in cancer cell lines is consistent with the profiles observed in patient samples; high mRNA expression is found in nearly all BCP-ALL cell lines and in subsets of T-ALL and AML cell lines (FIGS. 5A and 5B, https://portals.broadinstitute.org/ccle). Surprisingly, the cell line analysis also suggests that the pre-BCR is also expressed by some lymphoma cell lines, specifically Burkitt lymphoma and DLBCL (FIGS. 5A and 5B). Data suggest that patients spanning a spectrum of leukemia and lymphoma indications may benefit from pre-BCR-targeting therapy.

### Example 2. Generation of High Avidity Antibodies Against the Human Pre-BCR

Seven- to fourteen-week old female BALB/c mice were immunized with either the human VpreB polypeptide (amino acid residues 20-145 of SEQ ID NO:1) including a His-SUMO epitope tag on the N-terminus (Enquire BioReagents), or the human lambda-5 polypeptide (amino acid residues 45-213 of SEQ ID NO:3) including an N-terminal six-histidine epitope tag (ATGen). The mice were injected subcutaneously with 50 μg of immunogen in Freund's Complete Adjuvant per mouse. The immunized mice were boosted 14 days later with additional immunogen in Freund's Incomplete Adjuvant. Thereafter, for several weeks, the mice were boosted every 14 to 21 days with immunogen. Serum samples from the mice were periodically prepared from tail bleeds and tested by ELISA for the presence of antigen-specific antibodies. Mice with a significant antibody titer received a pre-fusion immunogen boost in phosphate-buffered saline four days prior to splenic fusion. The mice were sacrificed and the spleen cells were harvested and fused to a selected murine myeloma cell line P3/NSI/1-AG4-1 (NS-1) (ATCC No. TIB18) using 50% polyethylene glycol (Hybri-Max, Sigma). The fusions generated hybridoma cells which were plated in 96 well tissue culture plates containing HAT (hypoxanthine, aminopterin and thymidine) medium to inhibit proliferation of non-fused cells, myeloma hybrids and spleen hybrids. After hybridoma selection, the culture supernatants were assayed for binding to the human pre-B cell line, NALM-6, which expresses the pre-BCR.

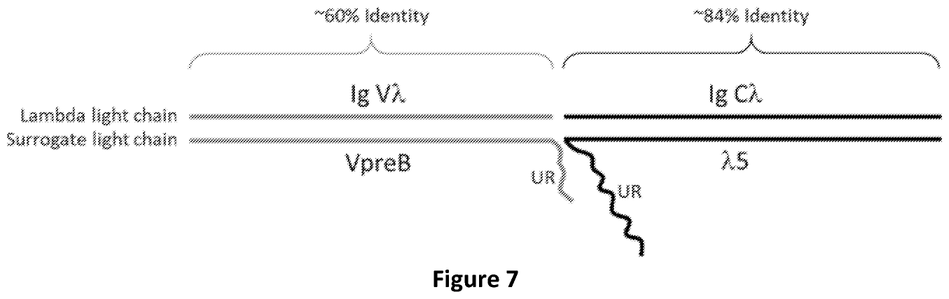

Hybridomas from three VpreB-immunized mice and one lambda-5-immunized mouse were screened. The supernatants of seven VpreB and 11 lambda-5 hybridoma cultures were found to contain antibodies that bind to NALM-6 cells. The conserved segments of the human SLC share significant homology with the Ig lambda light chain. VpreB is approximately 60% identical to lambda VL and lambda-5 bears approximately 84% identity to the lambda LC constant regions (FIG. 7). Therefore, to identify antibodies that bind the pre-BCR but do not cross-react with the Ig lambda light chain, the hybridoma supernatants were counter-screened on the human B cell lines Ramos, which expresses an Ig lambda light chain, and Raji, which expresses an Ig kappa light chain. All hybridomas expressing antibodies found to bind NALM-6 but not Ramos or Raji were subcloned by serial dilution methods.

### Example 3. Cloning and Sequence Analysis of High Affinity Antibodies Against Human Pre-BCR

The VH and VL were cloned from the pre-BCR-specific hybridomas described in Example 2 using RT-PCR and were sequenced. Mouse-human chimeric mAbs consisting of the mouse mAb VH and VL fused to the human IgG1 HC (SEQ ID NO:4) and kappa LC (SEQ ID NO:5) constant regions were produced as recombinant proteins in Expi293F cells. Transient transfection of Expi293F cells was conducted according to the manufacturer's protocol (Thermo Fisher Scientific) and recombinant antibodies were purified from 4-day culture supernatants using Protein A (GE Healthcare Life Sciences).

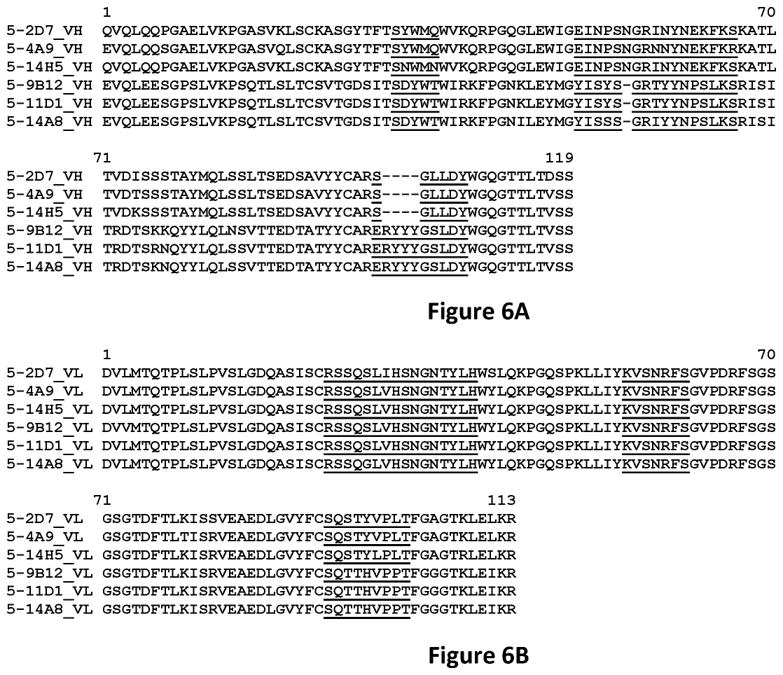

The sequences of the anti-VpreB VH and VL are shown in FIGS. 6A and 6B, respectively, and are included below. The CDRs and FRs of each variable region are provided in TABLES 4 and 5 below. The consensus CDR sequences are provided in TABLES 6-9 below.

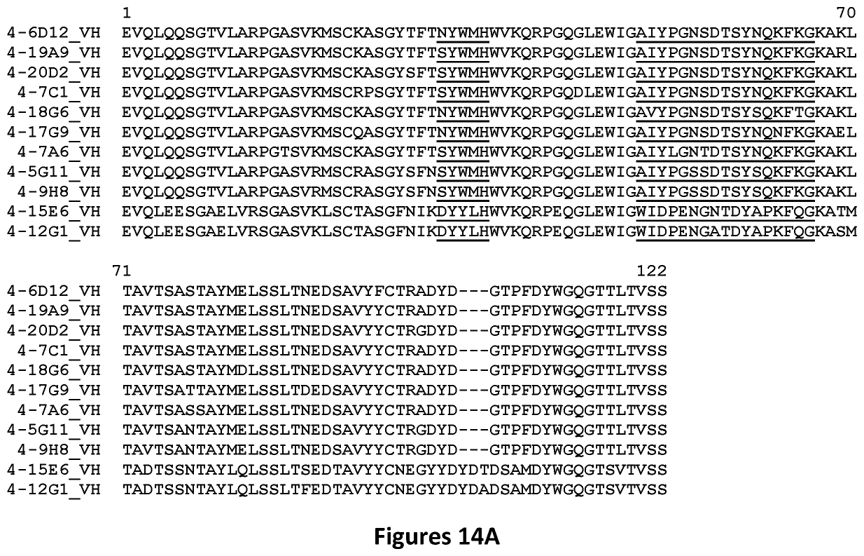

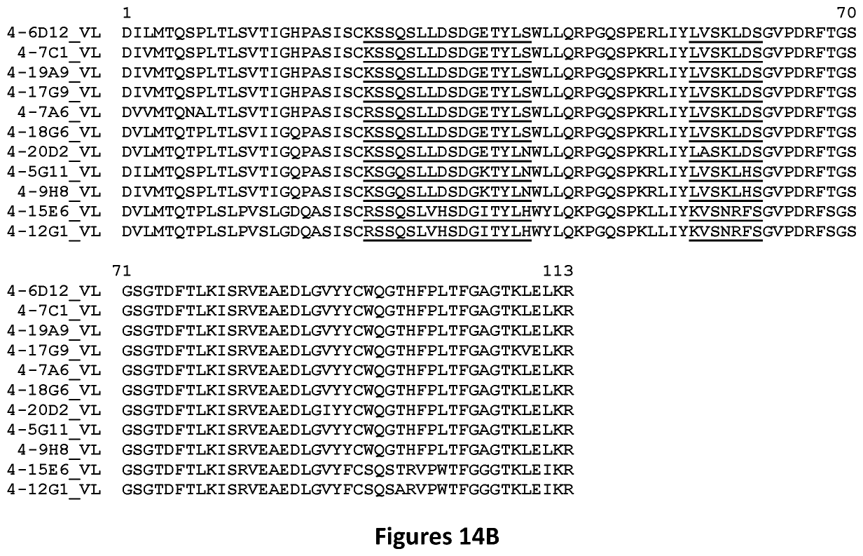

The sequences of the anti-lambda-5 VH and VL are shown in FIGS. 14A and 14B, respectively, and are included below. The CDRs and FRs of each variable region are provided in TABLES 10 and 11 below. The consensus CDR sequences are provided in TABLES 12-15 below.

Presented below is the VH sequence for each VpreB antibody. The Kabat CDRs are underlined.

VpreB Antibody Heavy Chain Variable Regions:
5-2D7 VH:
SEQ ID NO: 6
QVQLQQPGAELVKPGASVKLSCKASGYTFTSYWMQWVKQRPGQGLEWIG
EINPSNGRINYNEKFKSKATLTVDISSSTAYMQLSSLTSEDSAVYYCAR
SGLLDYWGQGTTLTDSS
5-4A9 VH:
SEQ ID NO: 7
EVQLQQSGAELVKPGASVQLSCKASGYTFTSYWMQWVKQRPGQGLEWIG
EINPSNGRNNYNEKFKRKATLTVDTSSSTAYMQLSSLTSEDSAVYYCAR
SGLLDYWGQGTTLTVSS
5-9B12 VH:
SEQ ID NO: 8
EVQLEESGPSLVKPSQTLSLTCSVTGDSITSDYWTWIRKFPGNKLEYMG
YISYSGRTYYNPSLKSRISITRDTSKKQYYLQLNSVTTEDTATYYCARE
RYYYGSLDYWGQGTTLTVSS
5-11D1 VH:
SEQ ID NO: 9
EVQLEESGPSLVKPSQTLSLTCSVTGDSITSDYWTWIRKFPGNKLEYMG
YISYSGRTYYNPSLKSRISITRDTSRNQYYLQLSSVTTEDTATYYCARE
RYYYGSLDYWGQGTTLTVSS
5-14A8 VH:
SEQ ID NO: 10
EVQLEESGPSLVKPSQTLSLTCSVTGDSITSDYWTWIRKFPGNILEYMG
YISSSGRIYYNPSLKSRISITRDTSKNQYYLQLSSVTTEDTATYYCARE
RYYYGSLDYWGQGTTLTVSS
5-14H5 VH:
SEQ ID NO: 11
QVQLQQPGAELVKPGASVKLSCKASGYTFTSNWMNWVKQRPGQGLEWIG
EINPSNGRINYNEKFKSKATLTVDKSSSTAYMQLSSLTSEDSAVYYCAR
SGLLDYWGQGTTLTVSS

TABLE 4
VpreB Antibody VH sequences (FR and CDR regions; Kabat)

| Antibody | HC FR1 | HC CDR1 |
|---|---|---|
| 5-2D7 | QVQLQQPGAELVKPGASVKLSCKASGYTFT | SYWMQ |
| | (SEQ ID NO: 18) | (SEQ ID NO: 19) |
| 5-4A9 | EVQLQQSGAELVKPGASVQLSCKASGYTFT | SYWMQ |
| | (SEQ ID NO: 25) | (SEQ ID NO: 19) |
| 5-9B12 | EVQLEESGPSLVKPSQTLSLTCSVTGDSIT | SDYWT |
| | (SEQ ID NO: 29) | (SEQ ID NO: 30) |
| 5-11D1 | EVQLEESGPSLVKPSQTLSLTCSVTGDSIT | SDYWT |
| | (SEQ ID NO: 29) | (SEQ ID NO: 30) |
| 5-14A8 | EVQLEESGPSLVKPSQTLSLTCSVTGDSIT | SDYWT |
| | (SEQ ID NO: 29) | (SEQ ID NO: 30) |
| 5-14H5 | QVQLQQPGAELVKPGASVKLSCKASGYTFT | SNWMN |
| | (SEQ ID NO: 18) | (SEQ ID NO: 39) |
| Antibody | HC FR2 | HC CDR2 |
| 5-2D7 | WVKQRPGQGLEWIG | EINPSNGRINYNEKFKS |
| | (SEQ ID NO: 20) | (SEQ ID NO: 21) |
| 5-4A9 | WVKQRPGQGLEWIG | EINPSNGRNNYNEKFKR |
| | (SEQ ID NO: 20) | (SEQ ID NO: 26) |
| 5-9B12 | WIRKFPGNKLEYMG | YISYSGRTYYNPSLKS |
| | (SEQ ID NO: 31) | (SEQ ID NO: 32) |
| 5-11D1 | WIRKFPGNKLEYMG | YISYSGRTYYNPSLKS |
| | (SEQ ID NO: 31) | (SEQ ID NO: 32) |
| 5-14A8 | WIRKFPGNILEYMG | YISSSGRIYYNPSLKS |
| | (SEQ ID NO: 36) | (SEQ ID NO: 37) |
| 5-14H5 | WVKQRPGQGLEWIG | EINPSNGRINYNEKFKS |
| | (SEQ ID NO: 20) | (SEQ ID NO: 21) |
| Antibody | HC FR3 | HC CDR3 |
| 5-2D7 | KATLTVDISSSTAYMQLSSLTSEDSAVYYCAR | SGLLDY |
| | (SEQ ID NO: 22) | (SEQ ID NO: 23) |
| 5-4A9 | KATLTVDTSSSTAYMQLSSLTSEDSAVYYCAR | SGLLDY |
| | (SEQ ID NO: 27) | (SEQ ID NO: 23) |
| 5-9B12 | RISITRDTSKKQYYLQLNSVTTEDTATYYCAR | ERYYYGSLDY |
| | (SEQ ID NO: 33) | (SEQ ID NO: 34) |
| 5-11D1 | RISITRDTSRNQYYLQLSSVTTEDTATYYCAR | ERYYYGSLDY |
| | (SEQ ID NO: 35) | (SEQ ID NO: 34) |
| 5-14A8 | RISITRDTSKNQYYLQLSSVTTEDTATYYCAR | ERYYYGSLDY |
| | (SEQ ID NO: 38) | (SEQ ID NO: 34) |
| 5-14H5 | KATLTVDKSSSTAYMQLSSLTSEDSAVYYCAR | SGLLDY |
| | (SEQ ID NO: 40) | (SEQ ID NO: 23) |

| Antibody | HC FR4 |
|---|---|
| 5-2D7 | WGQGTTLTDSS |
| | (SEQ ID NO: 24) |
| 5-4A9 | WGQGTTLTVSS |
| | (SEQ ID NO: 28) |
| 5-9B12 | WGQGTTLTVSS |
| | (SEQ ID NO: 28) |
| 5-11D1 | WGQGTTLTVSS |
| | (SEQ ID NO: 28) |
| 5-14A8 | WGQGTTLTVSS |
| | (SEQ ID NO: 28) |
| 5-14H5 | WGQGTTLTVSS |
| | (SEQ ID NO: 28) |

Presented below is the VL sequence for each VpreB antibody. The Kabat CDRs are underlined.

VpreB Antibody Light Chain Variable Regions:
5-2D7 VL:
SEQ ID NO: 12
DVLMTQTPLSLPVSLGDQASISCRSSQSLIHSNGNTYLHWSLQKPGQSP
KLLIYKVSNRFSGVPDRFSGSGSGTDFTLKISSVEAEDLGVYFC
SQSTYVPLTFGAGTKLELKR
5-4A9 VL:
SEQ ID NO: 13
DVLMTQTPLSLPVSLGDQASISCRSSQSLVHSNGNTYLHWYLQKPGQSP
KLLIYKVSNRFSGVPDRFSGSGSGTDFTLTISRVEAEDLGVYFC
SQSTYVPLTFGAGTKLELKR
5-9B12 VL:
SEQ ID NO: 14
DVVMTQTPLSLPVSLGDQASISCRSSQSLVHSNGNTYLHWYLQKPGQSP
KLLIYKVSNRFSGVPDRFSGSGSGTDFTLKISRVEAEDLGVYFC
SQTTHVPPTFGGGTKLEIKR
5-11D1 VL:
SEQ ID NO: 15
DVLMTQTPLSLPVSLGDQASISCRSSQSLVHSNGNTYLHWYLQKPGQSP
KLLIYKVSNRFSGVPDRFSGSGSGTDFTLKISRVEAEDLGVYFC
SQTTHVPPTFGGGTKLEIKR
5-14A8 VL:
SEQ ID NO: 16
DVLMTQTPLSLPVSLGDQASISCRSSQGLVHSNGNTYLHWYLQKPGQSP
KLLIYKVSNRFSGVPDRFSGSGSGTDFTLKISRVEAEDLGVYFC
SQTTHVPPTFGGGTKLEIKR
5-14H5 VL:
SEQ ID NO: 17
DVLMTQTPLSLPVSLGDQASISCRSSQSLVHSNGNTYLHWYLQKPGQSP
KLLIYKVSNRFSGVPDRFSGSGSGTDFTLKISRVEAEDLGVYFC
SQSTYLPLTFGAGTRLELKR

TABLE 5
VpreB Antibody VL sequences
(FR and CDR regions; Kabat)

| Antibody | LC FR1 | LC CDR1 |
|---|---|---|
| 5-2D7 | DVLMTQTPLSLPVSL | RSSQSLIHSNGNTYLH |
| | GDQASISC | (SEQ ID NO: 42) |
| | (SEQ ID NO: 41) | |
| 5-4A9 | DVLMTQTPLSLPVSL | RSSQSLVHSNGNTYLH |
| | GDQASISC | (SEQ ID NO: 48) |
| | (SEQ ID NO: 41) | |
| 5-9B12 | DVVMTQTPLSLPVSL | RSSQSLVHSNGNTYLH |
| | GDQASISC | (SEQ ID NO: 48) |
| | (SEQ ID NO: 51) | |
| 5-11D1 | DVLMTQTPLSLPVSL | RSSQSLVHSNGNTYLH |
| | GDQASISC | (SEQ IDNO: 48) |
| | (SEQ ID NO: 41) | |
| 5-14A8 | DVLMTQTPLSLPVSL | RS SQGLVHSNGNTYLH |
| | GDQASISC | (SEQ ID NO: 55) |
| | (SEQ ID NO: 41) | |
| 5-14H5 | DVLMTQTPLSLPVSL | RSSQSLVHSNGNTYLH |
| | GDQASISC | (SEQ ID NO: 48) |
| | (SEQ ID NO: 41) | |
| Antibody | LC FR2 | LC CDR2 |
| 5-2D7 | WSLQKPGQSPKLLIY | KVSNRFS |
| | (SEQ ID NO: 43) | (SEQ ID NO: 44) |
| 5-4A9 | WYLQKPGQSPKLLIY | KVSNRFS |
| | (SEQ ID NO: 49) | (SEQ ID NO: 44) |
| 5-9B12 | WYLQKPGQSPKLLIY | KVSNRFS |
| | (SEQ ID NO: 49) | (SEQ ID NO: 44) |
| 5-11D1 | WYLQKPGQSPKLLIY | KVSNRFS |
| | (SEQ ID NO: 49) | (SEQ ID NO: 44) |
| 5-14A8 | WYLQKPGQSPKLLIY | KVSNRFS |
| | (SEQ ID NO: 49) | (SEQ ID NO: 44) |
| 5-14H5 | WYLQKPGQSPKLLIY | KVSNRFS |
| | (SEQ ID NO: 49) | (SEQ ID NO: 44) |
| Antibody | LC FR3 | LC CDR3 |
| 5-2D7 | GVPDRFSGSGSGTDF | SQSTYVPLT |
| | TLKISSVEAEDLGVY | (SEQ ID NO: 46) |
| | FC | |
| | (SEQ ID NO: 45) | |
| 5-4A9 | GVPDRFSGSGSGTDF | SQSTYVPLT |
| | TLTISRVEAEDLGVY | (SEQ ID NO: 46) |
| | FC | |
| | (SEQ ID NO: 50) | |
| 5-9B12 | GVPDRFSGSGSGTDF | SQTTHVPPT |
| | TLKISRVEAEDLGVY | (SEQ ID NO: 53) |
| | FC | |
| | (SEQ ID NO: 52) | |
| 5-11D1 | GVPDRFSGSGSGTDF | SQTTHVPPT |
| | TLKISRVEAEDLGVY | (SEQ ID NO: 53) |
| | FC | |
| | (SEQ ID NO: 52) | |
| 5-14A8 | GVPDRFSGSGSGTDF | SQTTHVPPT |
| | TLKISRVEAEDLGVY | (SEQ ID NO: 53) |
| | FC | |
| | (SEQ ID NO: 52) | |
| 5-14H5 | GVPDRFSGSGSGTDF | SQSTYLPLT |
| | TLKISRVEAEDLGVY | (SEQ ID NO: 56) |
| | FC | |
| | (SEQ ID NO: 52) | |
| Antibody | LC FR4 | |
| 5-2D7 | FGAGTKLELKR | |
| | (SEQ ID NO: 47) | |
| 5-4A9 | FGAGTKLELKR | |
| | (SEQ ID NO: 47) | |
| 5-9B12 | FGGGTKLEIKR | |
| | (SEQ ID NO: 54) | |
| 5-11D1 | FGGGTKLEIKR | |
| | (SEQ ID NO: 54) | |
| 5-14A8 | FGGGTKLEIKR | |
| | (SEQ ID NO: 54) | |
| 5-14H5 | FGAGTRLELKR | |
| | (SEQ ID NO: 57) | |

TABLE 6
Consensus Sequences for Group IA
VpreB Antibody VH CDRs

| Antibody | Region | Sequence |
|---|---|---|
| 5-2D7 | HC-CDR1 | SYWMQ |
| | | (SEQ ID NO: 19) |
| 5-4A9 | HC-CDR1 | SYWMQ |
| | | (SEQ ID NO: 19) |
| 5-14H5 | HC-CDR1 | SNWMN |
| | | (SEQ ID NO: 39) |
| Consensus | HC-CDR1 | SXWMX |
| | | (SEQ ID NO: 58) |
| | | wherein X at |
| | | position 2 |
| | | is Y or N; |
| | | and wherein |
| | | X at position |
| | | 5 is |
| | | Q or N |
| 5-2D7 | HC-CDR2 | EINPSNGRINYNEKFKS |
| | | (SEQ ID NO: 21) |
| 5-4A9 | HC-CDR2 | EINPSNGRNNYNEKFKR |
| | | (SEQ ID NO: 26) |
| 5-14H5 | HC-CDR2 | EINPSNGRINYNEKFKS |
| | | (SEQ ID NO: 21) |
| Consensus | HC-CDR2 | EINPSNGRXNYNEKFKX |
| | | (SEQ ID NO: 59) |
| | | wherein X at |
| | | position 9 |
| | | is I or N; |
| | | and wherein X at |
| | | position 17 is |
| | | S or R |
| 5-2D7 | HC-CDR3 | SGLLDY |
| | | (SEQ ID NO: 23) |
| 5-4A9 | HC-CDR3 | SGLLDY |
| | | (SEQ ID NO: 23) |
| 5-14H5 | HC-CDR3 | SGLLDY |
| | | (SEQ ID NO: 23) |
| Consensus | HC-CDR3 | SGLLDY |
| | | (SEQ ID NO: 23) |

TABLE 7
Consensus Sequences for Group IA
VpreB Antibody VL CDRs

| Antibody | Region | Sequence |
|---|---|---|
| 5-2D7 | LC-CDR1 | RSSQSLIHSNGNTYLH |
| | | (SEQ ID NO: 42) |
| 5-4A9 | LC-CDR1 | RSSQSLVHSNGNTYLH |
| | | (SEQ ID NO: 48) |
| 5-14H5 | LC-CDR1 | RSSQSLVHSNGNTYLH |
| | | (SEQ ID NO: 48) |
| Consensus | LC-CDR1 | RSSQSLXHSNGNTYLH |
| | | (SEQ ID NO: 60) |
| | | wherein X at |
| | | position 7 |
| | | is I or V |
| 5-2D7 | LC-CDR2 | KVSNRFS |
| | | (SEQ ID NO: 44) |
| 5-4A9 | LC-CDR2 | KVSNRFS |
| | | (SEQ ID NO: 44) |
| 5-14H5 | LC-CDR2 | KVSNRFS |
| | | (SEQ ID NO: 44) |
| Consensus | LC-CDR2 | KVSNRFS |
| | | (SEQ ID NO: 44) |
| 5-2D7 | LC-CDR3 | SQSTYVPLT |
| | | (SEQ ID NO: 46) |
| 5-4A9 | LC-CDR3 | SQSTYVPLT |
| | | (SEQ ID NO: 46) |
| 5-14H5 | LC-CDR3 | SQSTYLPLT |
| | | (SEQIDNO: 53) |
| Consensus | LC-CDR3 | SQSTYXPLT |
| | | (SEQ ID NO: 61) |
| | | wherein X at |
| | | position 6 is |
| | | V or L |

TABLE 8
Consensus Sequences for Group IB
VpreB Antibody VH CDRs

| Antibody | Region | Sequence |
|---|---|---|
| 5-9B12 | HC-CDR1 | SDYWT |
| | | (SEQ ID NO: 30) |
| 5-11D1 | HC-CDR1 | SDYWT |
| | | (SEQ ID NO: 30) |
| 5-14A8 | HC-CDR1 | SDYWT |
| | | (SEQ ID NO: 30) |
| Consensus | HC-CDR1 | SDYWT |
| | | (SEQ ID NO: 30) |
| 5-9B12 | HC-CDR2 | YISYSGRTYYNPSLKS |
| | | (SEQ ID NO: 32) |
| 5-11D1 | HC-CDR2 | YISYSGRTYYNPSLKS |
| | | (SEQ ID NO: 32) |
| 5-14A8 | HC-CDR2 | YISSSGRIYYNPSLKS |
| | | (SEQ ID NO: 37) |
| Consensus | HC-CDR2 | YISXSGRXYYNPSLKS |
| | | (SEQ ID NO: 62) |
| | | wherein X at |
| | | position 4 is |
| | | Y or S; and wherein |
| | | X at position 8 |
| | | is T or I |
| 5-9B12 | HC-CDR3 | ERYYYGSLDY |
| | | (SEQ ID NO: 34) |
| 5-11D1 | HC-CDR3 | ERYYYGSLDY |
| | | (SEQ ID NO: 34) |
| 5-14A8 | HC-CDR3 | ERYYYGSLDY |
| | | (SEQ ID NO: 34) |
| Consensus | HC-CDR3 | ERYYYGSLDY |
| | | (SEQ ID NO: 34) |

TABLE 9
Consensus Sequences for Group IB
VpreB Antibody VL CDRs

| Antibody | Region | Sequence |
|---|---|---|
| 5-9B12 | LC-CDR1 | RSSQSLVHSNGNTYLH |
| | | (SEQ ID NO: 48) |
| 5-11D1 | LC-CDR1 | RSSQSLVHSNGNTYLH |
| | | (SEQ ID NO: 48) |
| 5-14A8 | LC-CDR1 | RSSQGLVHSNGNTYLH |
| | | (SEQ ID NO: 55) |
| Consensus | LC-CDR1 | RSSQXLVHSNGNTYLH |
| | | (SEQ ID NO: 63) |
| | | wherein X at |
| | | position 5 is |
| | | S or G |
| 5-9B12 | LC-CDR2 | KVSNRFS |
| | | (SEQ ID NO: 44) |
| 5-11D1 | LC-CDR2 | KVSNRFS |
| | | (SEQ ID NO: 44) |
| 5-14A8 | LC-CDR2 | KVSNRFS |
| | | (SEQ ID NO: 44) |
| Consensus | LC-CDR2 | KVSNRFS |
| | | (SEQ ID NO: 44) |
| 5-9B12 | LC-CDR3 | SQTTHVPPT |
| | | (SEQ ID NO: 53) |
| 5-11D1 | LC-CDR3 | SQTTHVPPT |
| | | (SEQ ID NO: 53) |
| 5-14A8 | LC-CDR3 | SQTTHVPPT |
| | | (SEQ ID NO: 53) |
| Consensus | LC-CDR3 | SQTTHVPPT |
| | | (SEQ ID NO: 53) |

DNA Encoding Mouse VpreB mAb Heavy and Light
Chain Variable Regions:
SEQ ID NO: 64: DNA encoding 5-2D7 VH
CAGGTCCAACTGCAGCAGCCTGGGGCTGAACTGGT
GAAGCCTGGGGCTTCAGTGAAGCTGTCCTGCAAGG
CTTCTGGCTACACCTTCACCAGCTACTGGATGCAG
TGGGTGAAGCAGAGGCCTGGACAAGGCCTTGAGTG
GATTGGAGAGATTAATCCTAGCAACGGTCGTATTA
ACTACAATGAGAAGTTCAAGAGCAAGGCCACACTT
ACTGTAGACATATCGTCCAGCACAGCCTACATGCA
ACTCAGCAGTCTGACATCTGAGGACTCTGCGGTCT
ATTACTGTGCAAGATCGGGGCTCCTTGACTACTGG
GGCCAAGGCACCACTCTCACAGACTCCTCA
SEQ ID NO: 65: DNA encoding 5-4A9 VH
GAGGTCCAGCTGCAACAGTCTGGGGCTGAACTGGT
GAAGCCTGGGGCTTCAGTGCAGCTGTCCTGCAAGG
CTTCTGGCTACACCTTCACCAGCTACTGGATGCAG
TGGGTGAAACAGAGGCCTGGACAAGGCCTTGAGTG
GATTGGAGAGATTAATCCTAGCAACGGTCGCAATA
ATTACAATGAGAAGTTCAAGAGAAAGGCCACACTT
ACTGTTGACACATCCTCCAGCACAGCCTACATGCA
ACTCAGCAGCCTGACATCTGAGGACTCTGCGGTCT
ATTACTGTGCAAGATCGGGGCTCCTTGACTACTGG
GGCCAAGGCACCACTCTCACAGTCTCCTCA
SEQ ID NO: 66: DNA encoding 5-9B12 VH
GAGGTGCAGCTGGAGGAGTCAGGACCTAGCCTCGT
GAAACCTTCTCAGACTCTGTCCCTCACCTGTTCTG
TCACTGGCGACTCCATCACCAGTGATTACTGGACC
TGGATCCGGAAATTCCCAGGGAATAAACTTGAGTA
CATGGGGTACATAAGCTACAGTGGTAGAACTTACT
ACAATCCATCTCTCAAAAGTCGAATCTCCATCACT
CGAGACACATCCAAGAAGCAGTACTACCTGCAGTT
GAATTCTGTGACAACTGAGGACACAGCCACATATT
ACTGTGCAAGAGAGCGTTATTACTACGGTAGTCTT
GACTACTGGGGCCAAGGCACCACTCTCACAGTCTC
CTCA
SEQ ID NO: 67: DNA encoding 5-11D1 VH
GAGGTGCAGCTGGAGGAGTCAGGACCTAGCCTCGT
GAAACCTTCTCAGACTCTGTCCCTCACCTGTTCTG
TCACTGGCGACTCCATCACCAGTGATTACTGGACC
TGGATCCGGAAATTCCCAGGGAATAAACTTGAGTA
CATGGGGTACATAAGCTACAGTGGTAGAACTTACT
ACAATCCATCTCTCAAAAGTCGAATCTCCATCACT
CGAGACACATCCAGGAACCAGTACTACCTGCAGTT
GAGTTCTGTGACTACTGAGGACACAGCCACATATT
ACTGTGCAAGAGAGCGTTATTACTACGGTAGTCTT
GACTACTGGGGCCAGGGCACCACTCTCACAGTCTC
CTCA
SEQ ID NO: 68: DNA encoding 5-14A8 VH
GAGGTGCAGCTGGAGGAGTCAGGACCTAGCCTCGT
GAAACCTTCTCAGACTCTGTCCCTCACCTGTTCTG
TCACTGGCGACTCCATCACCAGTGATTACTGGACC
TGGATCCGGAAATTCCCAGGGAATATACTTGAGTA
CATGGGGTACATAAGCTCCAGTGGTAGGATTTATT
ACAATCCATCTCTCAAAAGTCGAATCTCCATCACT
CGAGACACATCCAAGAACCAGTACTACCTGCAGTT
GAGTTCTGTGACTACTGAGGACACAGCCACATATT
ACTGTGCAAGAGAGCGTTATTACTACGGTAGTCTT
GACTACTGGGGCCAAGGCACCACTCTCACAGTCTC
CTCA
SEQ ID NO: 69: DNA encoding 5-14H5 VH
CAGGTCCAACTGCAGCAGCCTGGGGCTGAACTGGT
GAAGCCTGGGGCTTCAGTGAAGCTGTCCTGCAAGG
CTTCTGGCTACACCTTCACCAGCAACTGGATGAAC
TGGGTGAAGCAGAGGCCTGGACAAGGCCTTGAGTG
GATTGGAGAGATTAATCCTAGCAACGGTCGTATTA
ATTACAATGAGAAGTTCAAGAGCAAGGCCACACTT
ACTGTGGACAAATCCTCCAGCACAGCCTACATGCA
ACTCAGCAGCCTGACATCTGAGGACTCTGCGGTCT
ATTACTGTGCAAGATCGGGGCTCCTTGACTACTGG
GGCCAAGGCACCACTCTCACAGTCTCCTCA
SEQ ID NO: 70: DNA encoding 5-2D7 VL
GATGTTTTGATGACCCAAACTCCACTCTCCCTGCC
TGTCAGTCTTGGAGATCAAGCCTCCATCTCTTGCA
GATCTAGTCAGAGCCTTATACACAGTAATGGAAAC
ACCTATTTACATTGGTCCCTGCAGAAGCCAGGCCA
GTCTCCAAAGCTCCTGATCTACAAAGTTTCCAACC
GATTTTCTGGGGTCCCAGACAGGTTCAGTGGCAGT
GGATCAGGGACAGATTTCACACTCAAGATCAGCAG
CGTGGAGGCTGAGGATCTGGGAGTTTATTTCTGCT
CTCAAAGTACATATGTCCCGCTCACGTTCGGTGCT
GGGACCAAGCTGGAGCTGAAACGG
SEQ ID NO: 71: DNA encoding 5-4A9 VL
GATGTTTTGATGACCCAAACTCCACTCTCCCTGCC
TGTCAGTCTTGGAGATCAAGCCTCCATCTCTTGCA
GATCTAGTCAGAGCCTTGTACACAGTAATGGCAAC
ACCTATTTACATTGGTACCTGCAGAAGCCAGGCCA
GTCTCCAAAGCTCCTGATCTACAAAGTTTCTAACC
GATTTTCTGGGGTCCCAGACAGGTTCAGTGGCAGT
GGATCAGGGACAGATTTCACACTCACGATCAGCAG
AGTGGAGGCTGAGGATCTGGGAGTTTATTTCTGCT
CTCAAAGTACATATGTCCCGCTCACGTTCGGTGCT
GGGACCAAGCTGGAGCTGAAACGG
SEQ ID NO: 72: DNA encoding 5-9B12 VL
GATGTTGTGATGACCCAAACTCCACTCTCCCTGCC
TGTCAGTCTTGGAGATCAAGCCTCCATCTCTTGCA
GATCTAGTCAGAGCCTTGTACACAGTAATGGAAAC
ACCTATTTACATTGGTACCTGCAGAAGCCAGGCCA
GTCTCCAAAGCTCCTGATTTACAAAGTTTCCAACC
GATTTTCTGGGGTCCCAGACAGGTTCAGTGGCAGT
GGATCAGGGACAGATTTCACACTCAAGATCAGCAG
AGTGGAGGCTGAGGATCTGGGAGTTTATTTCTGCT
CTCAAACTACACATGTTCCTCCCACGTTCGGAGGG
GGGACCAAGCTGGAAATAAAACGG
SEQ ID NO: 73: DNA encoding 5-11D1 VL
GATGTTTTGATGACCCAAACTCCACTCTCCCTGCC
TGTCAGTCTTGGAGATCAAGCCTCCATCTCTTGCA
GATCTAGTCAGAGCCTTGTACACAGTAATGGAAAC
ACCTATTTACATTGGTACCTGCAGAAGCCAGGCCA
GTCTCCAAAGCTCCTGATCTACAAAGTTTCCAACC
GATTTTCTGGGGTCCCAGACAGGTTCAGTGGCAGT
GGATCAGGGACAGATTTCACACTCAAGATCAGCAG
AGTGGAGGCTGAGGATCTGGGAGTTTATTTCTGCT
CTCAAACTACACATGTTCCTCCCACGTTCGGAGGG
GGGACCAAGCTGGAAATAAAACGG
SEQ ID NO: 74: DNA encoding 5-14A8 VL
GATGTTTTGATGACCCAAACTCCACTCTCCCTGCC
TGTCAGTCTTGGAGATCAAGCCTCCATCTCTTGCA
GATCTAGTCAGGGCCTTGTACACAGTAATGGAAAC
ACCTATTTACATTGGTACCTGCAGAAGCCAGGCCA
GTCTCCAAAGCTCCTGATCTACAAAGTTTCCAACC
GATTTTCTGGGGTCCCAGACAGGTTCAGTGGCAGT
GGATCAGGGACAGATTTCACACTCAAGATCAGCAG
AGTGGAGGCTGAGGATCTGGGAGTTTATTTCTGCT
CTCAAACTACACATGTTCCTCCCACGTTCGGAGGG
GGGACCAAGCTGGAAATAAAACGG
SEQ ID NO: 75: DNA encoding 5-14H5 VL
GATGTTTTGATGACCCAAACTCCACTCTCCCTGCC
TGTCAGTCTTGGAGATCAAGCCTCCATCTCTTGCA
GATCTAGTCAGAGCCTTGTACACAGTAATGGAAAC
ACCTATTTACATTGGTACCTGCAGAAGCCAGGCCA
GTCTCCAAAGCTCCTGATCTACAAAGTTTCCAACC
GATTTTCTGGGGTCCCAGACAGGTTCAGTGGCAGT
GGATCAGGGACAGATTTCACACTCAAGATCAGCAG
AGTGGAGGCTGAGGATCTGGGAGTTTATTTCTGCT
CTCAAAGTACATATCTTCCGCTCACGTTCGGTGCT
GGGACCAGGCTGGAGCTGAAACGG

Presented below is the VH sequence for each lambda-5 antibody. The Kabat CDRs are underlined.

Lambda-5 Antibody Heavy Chain Variable Region:
4-15E6 VH:
SEQ ID NO: 76
EVQLEESGAELVRSGASVKLSCTASGFNIKDYYLHWVKQRPEQGLE
WIGWIDPENGNTDYAPKFQGKATMTADTSSNTAYLQLSSLTSEDTA
VYYCNEGYYDYDTDSAMDYWGQGTSVTVSS
4-6D12 VH:
SEQ ID NO: 94
EVQLQQSGTVLARPGASVKMSCKASGYTFTNYWMHWVKQRPGQGLE
WIGAIYPGNSDTSYNQKFKGKAKLTAVTSASTAYMELSSLTNEDSA
VYFCTRADYDGTPFDYWGQGTTLTVSS
4-5G11 VH:
SEQ ID NO: 95
EVQLQQSGTVLARPGASVRMSCRASGYSFNSYWMHWVKQRPGQGLE
WIGAIYPGSSDTSYSQKFKGKAKLTAVTSANTAYMELSSLTNEDSA
VYYCTRGDYDGTPFDYWGQGTTLTVSS
4-7A6 VH:
SEQ ID NO: 96
EVQLQQSGTVLARPGTSVKMSCKASGYTFTSYWMHWVKQRPGQGLE
WIGAIYLGNTDTSYNQKFKGKAKLTAVTSASSAYMELSSLTNEDSA
VYYCTRADYDGTPFDYWGQGTTLTVSS
4-7C1 VH:
SEQ ID NO: 97
EVQLQQSGTVLARPGASVKMSCRPSGYTFTSYWMHWVKQRPGQDLE
WIGAIYPGNSDTSYNQKFKGKAKLTAVTSASTAYMELSSLTNEDSA
VYYCTRADYDGTPFDYWGQGTTLTVSS
4-9H8 VH:
SEQ ID NO: 98
EVQLQQSGTVLARPGASVRMSCRASGYSFNSYWMHWVKQRPGQGLE
WIGAIYPGSSDTSYSQKFKGKAKLTAVTSANTAYMELSSLTNEDSA
VYYCTRGDYDGTPFDYWGQGTTLTVSS
4-12G1 VH:
SEQ ID NO: 99
EVQLEESGAELVRSGASVKLSCTASGFNIKDYYLHWVKQRPEQGLE
WIGWIDPENGATDYAPKFQGKASMTADTSSNTAYLQLSSLTFEDTA
VYYCNEGYYDYDADSAMDYWGQGTSVTVSS
4-17G9 VH:
SEQ ID NO: 100
EVQLQQSGTVLARPGASVKMSCQASGYTFTNYWMHWVKQRPGQGLE
WIGAIYPGNSDTSYNQNFKGKAELTAVTSATTAYMELSSLTDEDSA
VYYCTRADYDGTPFDYWGQGTTLTVSS
4-18G6 VH:
SEQ ID NO: 101
EVQLQQSGTVLARPGASVKMSCKASGYTFTNYWMHWVKQRPGQGLE
WIGAVYPGNSDTSYSQKFTGKAKLTAVTSASTAYMDLSSLTNEDSA
VYYCTRADYDGTPFDYWGQGTTLTVSS
4-19A9 VH:
SEQ ID NO: 102
EVQLQQSGTVLARPGASVKMSCKASGYTFTSYWMHWVKQRPGQGLE
WIGAIYPGNSDTSYNQKFKGKARLTAVTSASTAYMELSSLTNEDSA
VYYCTRADYDGTPFDYWGQGTTLTVSS
4-20D2 VH:
SEQ ID NO: 103
EVQLQQSGTVLARPGASVKMSCKASGYSFTSYWMHWVKQRPGQGLE
WIGAIYPGNSDTSYNQKFKGKAKLTAVTSASTAYMELSSLTNEDSA
VYYCTRGDYDGTPFDYWGQGTTLTVSS

TABLE 10
Lambda-5 Antibody VH Sequences
(FR and CDR regions; Kabat)

| Antibody | HC FR1 | HC CDR1 |
|---|---|---|
| 4-15E6 | EVQLEESGAELVRSG | DYYLH |
| | ASVKLSCTASGFNIK | (SEQ ID NO: 79) |
| | (SEQ ID NO: 78) | |
| 4-6D12 | EVQLQQSGTVLARPG | NYWMH |
| | ASVKMSCKASGYTFT | (SEQ ID NO: 115) |
| | (SEQ ID NO: 114) | |
| 4-5G11 | EVQLQQSGTVLARPG | SYWMH |
| | ASVRMSCRASGYSFN | (SEQ ID NO: 117) |
| | (SEQ ID NO: 116) | |
| 4-7A6 | EVQLQOSGTVLARPG | SYWMH |
| | TSVKMSCKASGYTFT | (SEQ ID NO: 117) |
| | (SEQ ID NO: 118) | |
| 4-7C1 | EVQLQQSGTVLARPG | SYWMH |
| | ASVKMSCRPSGYTFT | (SEQ ID NO: 117) |
| | (SEQ ID NO: 119) | |
| 4-9H8 | EVQLQQSGTVLARPG | SYWMH |
| | ASVRMSCRASGYSFN | (SEQ ID NO: 117) |
| | (SEQ ID NO: 116) | |
| 4-12G1 | EVQLEESGAELVRSG | DYYLH |
| | ASVKLSCTASGFNIK | (SEQ ID N0 79) |
| | (SEQ ID NO: 78) | |
| 4-17G9 | EVQLQQSGTVLARPG | NYWMH |
| | ASVKMSCQASGYTFT | (SEQ ID NO: 115) |
| | (SEQ ID NO: 120) | |
| 4-18G6 | EVQLQQSGTVLARPG | NYWMH |
| | ASVKMSCKASGYTFT | (SEQ ID NO: 115) |
| | (SEQ ID NO: 114) | |
| 4-19A9 | EVQLQQSGTVLARPG | SYWMH |
| | ASVKMSCKASGYTFT | (SEQ ID NO: 117) |
| | (SEQ ID NO: 114) | |
| 4-20D2 | EVQLQQSGTVLARPG | SYWMH |
| | ASVKMSCKASGYSFT | (SEQ ID NO: 117) |
| | (SEQ ID NO: 121) | |
| Antibody | HC FR2 | HC CDR2 |
| 4-15E6 | WVKQRPEQGLEWIG | WIDPENGNTDYAPKFQG |
| | (SEQ ID NO: 80) | (SEQ ID NO: 81) |
| 4-6D12 | WVKQRPGQGLEWIG | AIYPGNSDTSYNQKFKG |
| | (SEQ ID NO: 122) | (SEQ ID NO: 123) |
| 4-5G11 | WVKQRPGQGLEWIG | AIYPGSSDTSY SQKFKG |
| | (SEQ ID NO: 122) | (SEQ ID NO: 124) |
| 4-7A6 | WVKQRPGQGLEWIG | AIYLGNTDTSYNQKFKG |
| | (SEQ ID NO: 122) | (SEQ ID NO: 125) |
| 4-7C1 | WVKQRPGQDLEWIG | AIYPGNSDTSYNQKFKG |
| | (SEQ ID NO: 126) | (SEQ ID NO: 123) |
| 4-9H8 | WVKQRPGQGLEWIG | AIYPGSSDTSYSQKFKG |
| | (SEQ ID NO: 122) | (SEQ ID NO: 124) |
| 4-12G1 | WVKQRPEQGLEWIG | WIDPENGATDYAPKFQG |
| | (SEQ ID NO: 80) | (SEQ ID NO: 127) |
| 4-17G9 | WVKQRPGQGLEWIG | AIYPGNSDTSYNQNFKG |
| | (SEQ ID NO: 122) | (SEQ ID NO: 128) |
| 4-18G6 | WVKQRPGQGLEWIG | AVYPGNSDTSYSQKFTG |
| | (SEQ ID NO: 122) | (SEQ ID NO: 129) |
| 4-19A9 | WVKQRPGQGLEWIG | AIYPGNSDTSYNQKFKG |
| | (SEQ ID NO: 122) | (SEQ ID NO: 123) |
| 4-20D2 | WVKQRPGQGLEWIG | AIYPGNSDTSYNQKFKG |
| | (SEQ ID NO: 122) | (SEQ ID NO: 123) |
| Antibody | HC FR3 | HC CDR3 |
| 4-15E6 | KATMTADTSSNTAYL | GYYDYDTDSAMDY |
| | QLSSLTSEDTAVYYC | (SEQ ID NO: 83) |
| | NE | |
| | (SEQ ID NO: 82) | |
| 4-6D12 | KAKLTAVTSASTAYM | ADYDGTPFDY |
| | ELSSLTNEDSAVYFC | (SEQ ID NO: 131) |
| | TR | |
| | (SEQ ID NO: 130) | |
| 4-5G11 | KAKLTAVTSANTAYM | GDYDGTPFDY |
| | ELSSLTNEDSAVYYC | (SEQ ID NO: 133) |
| | TR | |
| | (SEQ ID NO: 132) | |
| 4-7A6 | KAKLTAVTSASSAYM | ADYDGTPFDY |
| | ELSSLTNEDSAVYYC | (SEQ ID NO: 131) |
| | TR | |
| | (SEQ ID NO: 134) | |
| 4-7C1 | KAKLTAVTSASTAYM | ADYDGTPFDY |
| | ELSSLTNEDSAVYYC | (SEQ ID NO: 131) |
| | TR | |
| | (SEQ ID NO: 135) | |
| 4-9H8 | KAKLTAVTSANTAYM | GDYDGTPFDY |
| | ELSSLTNEDSAVYYC | (SEQ ID NO: 133) |
| | TR | |
| | (SEQ ID NO: 132) | |
| 4-12G1 | KASMTADTSSNTAYL | GYYDYDADSAMDY |
| | QLSSLTFEDTAVYYC | (SEQ ID NO: 137) |
| | NE | |
| | (SEQ ID NO: 136) | |
| 4-17G9 | KAELTAVTSATTAYM | ADYDGTPFDY |
| | ELSSLTDEDSAVYYC | (SEQ ID NO: 131) |
| | TR | |
| | (SEQ ID NO: 138) | |
| 4-18G6 | KAKLTAVTSASTAYM | ADYDGTPFDY |
| | DLSSLTNEDAVYYCTR | (SEQ ID NO: 131) |
| | (SEQ ID NO: 139) | |
| 4-19A9 | KARLTAVTSASTAYME | ADYDGTPFDY |
| | LSSLTNEDSAVYYCTR | (SEQ ID NO: 131) |
| | (SEQ ID NO: 140) | |
| 4-20D2 | KAKLTAVTSASTAYME | GDYDGTPFDY |
| | LSSLTNEDSAVYYCTR | (SEQ ID NO: 133) |
| | (SEQ ID NO: 135) | |
| Antibody | HC FR4 | |
| 4-15E6 | WGQGTSVTVSS | |
| | (SEQ ID NO: 84) | |
| 4-6D12 | WGQGTTLTVSS | |
| | (SEQ ID NO: 141) | |
| 4-5G11 | WGQGTTLTVSS | |
| | (SEQ ID NO: 141) | |
| 4-7A6 | WGQGTTLTVSS | |
| | (SEQ ID NO: 141) | |
| 4-7C1 | WGQGTTLTVSS | |
| | (SEQ ID NO: 141) | |
| 4-9H8 | WGQGTTLTVSS | |
| | (SEQ ID NO: 141) | |
| 4-12G1 | WGQGTSVTVSS | |
| | (SEQ ID NO: 84) | |
| 4-17G9 | WGQGTTLTVSS | |
| | (SEQ ID NO: 141) | |
| 4-18G6 | WGQGTTLTVSS | |
| | (SEQ ID NO: 141) | |
| 4-19A9 | WGQGTTLTVSS | |
| | (SEQ ID NO: 141) | |
| 4-20D2 | WGQGTTLTVSS | |
| | (SEQ ID NO: 141) | |

Presented below is the VL sequence for each lambda-5 antibody. The Kabat CDRs are underlined.

Lambda-5 Antibody Light Chain Variable Region:
4-15E6 VL:
SEQ ID NO: 77
DVLMTQTPLSLPVSLGDQASISCRSSQSLVHSDGITYLHW
YLQKPGQSPKWYKVSNRFSGVPDRFSGSGSGTDFTLKISR
VEAEDLGVYFCSQSTRVPWTFGGGTKLEIKR
4-6D12 VL:
SEQ ID NO: 104
DILMTQSPLTLSVTIGHPASISCKSSQSLLDSDGETYLSW
LLQRPGQSPERLIYLVSKLDSGVPDRFTGSGSGTDFTLKI
SRVEAEDLGVYYCWQGTHFPLTFGAGTKLELKR
4-5G11 VL:
SEQ ID NO: 105
DILMTQSPLTLSVTIGQPASISCKSGQSLLDSDGKTYLNW
LLQRPGQSPKRLIYLVSKLHSGVPDRFTGSGSGTDFTLKI
SRVEAEDLGVYYCWQGTHFPLTFGAGTKLELKR
4-7A6 VL:
SEQ ID NO: 106
DVVMTQNALTLSVTIGHPASISCRSSQSLLDSDGETYLSW
LLQRPGQSPKRLIYLVSKLDSGVPDRFTGSGSGTDFTLKI
SRVEAEDLGVYYCWQGTHFPLTFGAGTKLELKR
4-7C1 VL:
SEQ ID NO: 107
DIVMTQSPLTLSVTIGHPASISCKSSQSLLDSDGETYLSW
LLQRPGQSPKRLIYLVSKLDSGVPDRFTGSGSGTDFTLKI
SRVEAEDLGVYYCWQGTHFPLTFGAGTKLELKR
4-9H8 VL:
SEQ ID NO: 108
DIVMTQSPLTLSVTIGQPASISCKSGQSLLDSDGKTYLNW
LLQRPGQSPKRLIYLVSKLHSGVPDRFTGSGSGTDFTLKI
SRVEAEDLGVYYCWQGTHFPLTFGAGTKLELKR
4-12G1 VL:
SEQ ID NO: 109
DVLMTQTPLSLPVSLGDQASISCRSSQSLVHSDGITYLHW
YLQKPGQSPKLLIYKVSNRFSGVPDRFSGSGSGTDFTLKI
SRVEAEDLGVYFCSQSARVPWTFGGGTKLEIKR
4-17G9 VL:
SEQ ID NO: 110
DIVMTQSPLTLSVTIGHPASISCKSSQSLLDSDGETYLSW
LLQRPGQSPKRLIYLVSKLDSGVPDRFTGSGSGTDFTLKI
SRVEAEDLGVYYCWQGTHFPLTFGAGTKVELKR
4-18G6 VL:
SEQ ID NO: 111
DVLMTQTPLTLSVIIGQPASISCKSSQSLLDSDGETYLSW
LLQRPGQSPKRLIYLVSKLDSGVPDRFTGSGSGTDFTLKI
SRVEAEDLGVYYCWQGTHFPLTFGAGTKLELKR
4-19A9 VL:
SEQ ID NO: 112
DIVMTQSPLTLSVTIGHPASISCKSSQSLLDSDGETYLSW
LLQRPGQSPKRLIYLVSKLDSGVPDRFTGSGSGTDFTLKI
SRVEAEDLGVYYCWQGTHFPLTFGAGTKLELKR
4-20D2 VL:
SEQ ID NO: 113
DVLMTQTPLTLSVTIGQPASISCKSSQSLLDSDGETYLNW
LLQRPGQSPKRLIYLASKLDSGVPDRFTGSGSGTDFTLKI
SRVEAEDLGIYYCWQGTHFPLTFGAGTKLELKR

TABLE 11
Lambda-5 Antibody VL Sequences
(FR and CDR regions; Kabat)

| Antibody | LC FR1 | LC CDR1 |
|---|---|---|
| 4-15E6 | DVLMTQTPLSLPVS | RSSQSLVHSDGITYLH |
| | LGDQASISC | (SEQ ID N0 86) |
| | (SEQ ID NO: 85) | |
| 4-6D12 | DILMTQSPLTLSVT | KSSQSLLDSDGETYLS |
| | IGHPASISC | (SEQ ID NO: 143) |
| | (SEQ ID NO: 142) | |
| 4-5G11 | DILMTQSPLTLSVT | KSGQSLLDSDGKTYLN |
| | IGQPASISC | (SEQ ID NO: 145) |
| | (SEQ ID NO: 144) | |
| 4-7A6 | DVVMTQNALTLSVT | RSSQSLLDSDGETYLS |
| | IGHPASISC | (SEQ ID NO: 147) |
| | (SEQ ID NO: 146) | |
| 4-7C1 | DIVMTQSPLTLSVT | KSSQSLLDSDGETYLS |
| | IGHPASISC | (SEQ ID NO: 143) |
| | (SEQ ID NO: 148) | |
| 4-9H8 | DIVMTQSPLTLSVT | KSGQSLLDSDGKTYLN |
| | IGQPASISC | (SEQ ID NO: 145) |
| | (SEQ ID NO: 149) | |
| 4-12G1 | DVLMTQTPLSLPVS | RSSQSLVHSDGITYLH |
| | LGDQASISC | (SEQ ID NO: 86) |
| | (SEQ ID NO: 85) | |
| 4-17G9 | DIVMTQSPLTLSVT | KSSQSLLDSDGETYLS |
| | IGHPASISC | (SEQ ID NO: 143) |
| | (SEQ ID NO: 148) | |
| 4-18G6 | DVLMTQTPLTLSVI | KSSQSLLDSDGETYLS |
| | IGQPASISC | (SEQ ID NO: 143) |
| | (SEQ ID NO: 150) | |
| 4-19A9 | DIVMTQSPLTLSVT | KSSQSLLDSDGETYLS |
| | IGHPASISC | (SEQ ID NO: 143) |
| | (SEQ ID NO: 148) | |
| 4-20D2 | DVLMTQTPLTLSVT | KSSQSLLDSDGETYLN |
| | IGQPASISC | (SEQ ID NO: 152) |
| | (SEQ ID NO: 151) | |
| Antibody | LC FR2 | LC CDR2 |
| 4-15E6 | WYLQKPGQSPKLLIY | KVSNRFS |
| | (SEQ ID NO: 87) | (SEQ ID NO: 88) |
| 4-6D12 | WLLQRPGQSPERLIY | LVSKLDS |
| | (SEQ ID NO: 153) | (SEQ ID NO: 154) |
| 4-5G11 | WLLQRPGQSPKRLIY | LVSKLHS |
| | (SEQ ID NO: 155) | (SEQ ID NO: 156) |
| 4-7A6 | WLLQRPGQSPKRLIY | LVSKLDS |
| | (SEQ ID NO: 155) | (SEQ ID NO: 154) |
| 4-7C1 | WLLQRPGQSPKRLIY | LVSKLDS |
| | (SEQ ID NO: 155) | (SEQ ID NO: 154) |
| 4-9H8 | WLLQRPGQSPKRLIY | LVSKLHS |
| | (SEQ ID NO: 155) | (SEQ ID NO: 156) |
| 4-12G1 | WYLQKPGQSPKLLIY | KVSNRFS |
| | (SEQ ID NO: 87) | (SEQ ID NO: 88) |
| 4-17G9 | WLLQRPGQSPKRLIY | LVSKLDS |
| | (SEQ ID NO: 155) | (SEQ ID NO: 154) |
| 4-18G6 | WLLQRPGQSPKRLIY | LVSKLDS |
| | (SEQ ID NO: 155) | (SEQ ID NO: 154) |
| 4-19A9 | WLLQRPGQSPKRLIY | LVSKLDS |
| | (SEQ ID NO: 155) | (SEQ ID NO: 154) |
| 4-20D2 | WLLQRPGQSPKRLIY | LASKLDS |
| | (SEQ ID NO: 155) | (SEQ ID NO: 157) |
| Antibody | LC FR3 | LC CDR3 |
| 4-15E6 | GVPDRFSGSGSGTDF | SQSTRVPWT |
| | TLKISRVEAEDLGVY | (SEQ ID NO: 90) |
| | FC | |
| | (SEQ ID NO: 89) | |
| 4-6D12 | GVPDRFTGSGSGTDF | WQGTHFPLT |
| | TLKISRVEAEDLGVY | (SEQ ID NO: 159) |
| | YC | |
| | (SEQ ID NO: 158) | |
| 4-5G11 | GVPDRFTGSGSGTDF | WQGTHFPLT |
| | TLKISRVEAEDLGVY | (SEQ ID NO: 159) |
| | YC | |
| | (SEQ ID NO: 158) | |
| 4-7A6 | GVPDRFTGSGSGTDF | WQGTHFPLT |
| | TLKISRVEAEDLGVY | (SEQ ID NO: 159) |
| | YC | |
| | (SEQ ID NO: 158) | |
| 4-7C1 | GVPDRFTGSGSGTDF | WQGTHFPLT |
| | TLKISRVEAEDLGVY | (SEQ ID N0159 |
| | YC | |
| | (SEQ ID NO: 158) | |
| 4-9H8 | GVPDRFTGSGSGTDF | WQGTHFPLT |
| | TLKISRVEAEDLGVY | (SEQ ID NO: 159) |
| | YC | |
| | (SEQ ID NO: 158) | |
| 4-12G1 | GVPDRFSGSGSGTDF | SQSARVPWT |
| | TLKISRVEAEDLGVY | (SEQ ID NO: 160) |
| | FC | |
| | (SEQ ID NO: 89 ) | |
| 4-17G9 | GVPDRFTGSGSGTDFT | WQGTHFPLT |
| | LKISRVEAEDLGVYYC | (SEQ ID NO: 159) |
| | (SEQ ID NO: 158) | |
| 4-18G6 | GVPDRFTGSGSGTDFT | WQGTHFPLT |
| | LKISRVEAEDLGVYYC | (SEQ ID NO: 159) |
| | (SEQ ID NO: 158) | |
| 4-19A9 | GVPDRFTGSGSGTDFT | WQGTHFPLT |
| | LKISRVEAEDLGVYYC | (SEQ ID NO: 159) |
| | (SEQ ID NO: 158) | |
| 4-20D2 | GVPDRFTGSGSGTDFT | WQGTHFPLT |
| | LKISRVEAEDLGIYYC | (SEQ ID NO: 159) |
| | (SEQ ID NO: 161) | |
| Antibody | LC FR4 | |
| 4-15E6 | FGGGTKLEIKR | |
| | (SEQ ID NO: 91) | |
| 4-6D12 | FGAGTKLELKR | |
| | (SEQ ID NO: 162) | |
| 4-5G11 | FGAGTKLELKR | |
| | (SEQ ID NO: 162) | |
| 4-7A6 | FGAGTKLELKR | |
| | (SEQ ID NO: 162) | |
| 4-7C1 | FGAGTKLELKR | |
| | (SEQ ID NO: 162) | |
| 4-9H8 | FGAGTKLELKR | |
| | (SEQ ID NO: 162) | |
| 4-12G1 | FGGGTKLEIKR | |
| | (SEQ ID NO: 91) | |
| 4-17G9 | FGAGTKVELKR | |
| | (SEQ ID NO: 163) | |
| 4-18G6 | FGAGTKLELKR | |
| | (SEQ ID NO: 162) | |
| 4-19A9 | FGAGTKLELKR | |
| | (SEQ ID NO: 162) | |
| 4-20D2 | FGAGTKLELKR | |
| | (SEQ ID NO: 162) | |

TABLE 12
Consensus Sequences for Group IA
Lambda-5 Antibody VH CDRs

| Antibody | Region | Sequence |
|---|---|---|
| 4-6D12 | HC-CDR1 | NYWMH |
| | | (SEQ ID NO: 115) |
| 4-5G11 | HC-CDR1 | SYWMH |
| | | (SEQ ID NO: 117) |
| 4-7A6 | HC-CDR1 | SYWMH |
| | | (SEQ ID NO: 117) |
| 4-7C1 | HC-CDR1 | SYWMH |
| | | (SEQ ID NO: 117) |
| 4-9H8 | HC-CDR1 | SYWMH |
| | | (SEQ ID NO: 117) |
| 4-17G9 | HC-CDR1 | NYWMH |
| | | (SEQ ID NO: 115) |
| 4-18G6 | HC-CDR1 | NYWMH |
| | | (SEQ ID NO: 115) |
| 4-19A9 | HC-CDR1 | SYWMH |
| | | (SEQ ID NO: 117) |
| 4-20D2 | HC-CDR1 | SYWMH |
| | | (SEQ ID NO: 117) |
| Consensus | HC-CDR1 | XYWMH |
| | | (SEQ ID NO: 164) |
| | | wherein X at position |
| | | 1 is N or S |
| 4-6D12 | HC-CDR2 | AIYPGNSDTSYNQKFKG |
| | | (SEQ ID NO: 123) |
| 4-5G11 | HC-CDR2 | AIYPGSSDTSYSQKFKG |
| | | (SEQ ID NO: 124) |
| 4-7A6 | HC-CDR2 | AIYLGNTDTSYNQKFKG |
| | | (SEQ ID NO: 125) |
| 4-7C1 | HC-CDR2 | AIYPGNSDTSYNQKFKG |
| | | (SEQ ID NO: 123) |
| 4-9H8 | HC-CDR2 | AIYPGSSDTSYSQKFKG |
| | | (SEQ ID NO: 124) |
| 4-17G9 | HC-CDR2 | AIYPGNSDTSYNQNFKG |
| | | (SEQ ID NO: 128) |
| 4-18G6 | HC-CDR2 | AVYPGNSDTSYSQKFTG |
| | | (SEQ ID N0129) |
| 4-19A9 | HC-CDR2 | AIYPGNSDTSYNQKFKG |
| | | (SEQ ID NO: 123) |
| 4-20D2 | HC-CDR2 | AIYPGNSDTSYNQKFKG |
| | | (SEQ ID NO: 123) |
| Consensus | HC-CDR2 | AXYXGXXDTSYXQXFXG |
| | | (SEQ ID NO: 165) |
| | | wherein X at position |
| | | 2 is I or V; |
| | | and |
| | | wherein X at position |
| | | 4 is P or L; |
| | | and |
| | | wherein X at position |
| | | 6 is N or S; |
| | | and |
| | | wherein X at position |
| | | 7 is S or T; |
| | | and |
| | | wherein X at position |
| | | 12 is N or S; |
| | | and |
| | | wherein X at position |
| | | 14 is K or N; |
| | | and |
| | | wherein X at position |
| | | 16 is K or T |
| 4-6D12 | HC-CDR3 | ADYDGTPFDY |
| | | (SEQ ID NO: 131) |
| 4-5G11 | HC-CDR3 | GDYDGTPFDY |
| | | (SEQ ID NO: 133) |
| 4-7A6 | HC-CDR3 | ADYDGTPFDY |
| | | (SEQ ID NO: 131) |
| 4-7C1 | HC-CDR3 | ADYDGTPFDY |
| | | (SEQ ID NO: 131) |
| 4-9H8 | HC-CDR3 | GDYDGTPFDY |
| | | (SEQ ID NO: 133) |
| 4-17G9 | HC-CDR3 | ADYDGTPFDY |
| | | (SEQ ID NO: 131) |
| 4-18G6 | HC-CDR3 | ADYDGTPFDY |
| | | (SEQ ID NO: 131) |
| 4-19A9 | HC-CDR3 | ADYDGTPFDY |
| | | (SEQ ID NO: 131) |
| 4-20D2 | HC-CDR3 | GDYDGTPFDY |
| | | (SEQ ID NO: 133) |
| Consensus | HC-CDR3 | XDYDGTPFDY |
| | | (SEQ ID NO: 166) |
| | | wherein X at position |
| | | 1 is A or G |

TABLE 13
Consensus Sequences for Group IA
Lambda-5 Antibody VL CDRs

| Antibody | Region | Sequence |
|---|---|---|
| 4-6D12 | LC-CDR1 | KSSQSLLDSDGETYLS |
| | | (SEQ ID NO: 143) |
| 4-5G11 | LC-CDR1 | KSGQSLLDSDGKTYLN |
| | | (SEQ ID NO: 145) |
| 4-7A6 | LC-CDR1 | RSSQSLLDSDGETYLS |
| | | (SEQ ID NO: 147) |
| 4-7C1 | LC-CDR1 | KSSQSLLDSDGETYLS |
| | | (SEQ ID NO: 143) |
| 4-9H8 | LC-CDR1 | KSGQSLLDSDGKTYLN |
| | | (SEQ ID NO: 145) |
| 4-17G9 | LC-CDR1 | KSSQSLLDSDGETYLS |
| | | (SEQ ID NO: 143) |
| 4-18G6 | LC-CDR1 | KSSQSLLDSDGETYLS |
| | | (SEQ ID NO: 143) |
| 4-19A9 | LC-CDR1 | KSSQSLLDSDGETYLS |
| | | (SEQ ID NO: 143) |
| 4-20D2 | LC-CDR1 | KSSQSLLDSDGETYLN |
| | | (SEQ ID NO: 152) |
| Consensus | LC-CDR1 | XSXQSLLDSDGXTYLX |
| | | (SEQ ID NO: 167) |
| | | wherein X at position |
| | | 1 is K or R; |
| | | and |
| | | wherein X at position |
| | | 3 is S or G; |
| | | wherein X at position |
| | | 12 is E or K; |
| | | wherein X at position |
| | | 16 is S or N |
| 4-6D12 | LC-CDR2 | LVSKLDS |
| | | (SEQ ID NO: 154) |
| 4-5G11 | LC-CDR2 | LVSKLHS |
| | | (SEQ ID NO: 156) |
| 4-7A6 | LC-CDR2 | LVSKLDS |
| | | (SEQ ID NO: 154) |
| 4-7C1 | LC-CDR2 | LVSKLDS |
| | | (SEQ ID NO: 154) |
| 4-9H8 | LC-CDR2 | LVSKLHS |
| | | (SEQ ID NO: 156) |
| 4-17G9 | LC-CDR2 | LVSKLDS |
| | | (SEQ ID NO: 154) |
| 4-18G6 | LC-CDR2 | LVSKLDS |
| | | (SEQ ID NO: 154) |
| 4-19A9 | LC-CDR2 | LVSKLDS |
| | | (SEQ ID NO: 154) |
| 4-20D2 | LC-CDR2 | LASKLDS |
| | | (SEQ ID NO: 157) |
| Consensus | LC-CDR2 | LXSKLXS |
| | | (SEQ ID NO: 168) |
| | | wherein X at position |
| | | 2 is V or A; |
| | | and |
| | | wherein X at position |
| | | 6 is D or H |
| 4-6D12 | LC-CDR3 | WQGTHFPLT |
| | | (SEQ ID NO: 159) |
| 4-5G11 | LC-CDR3 | WQGTHFPLT |
| | | (SEQ ID NO: 159) |
| 4-7A6 | LC-CDR3 | WQGTHFPLT |
| | | (SEQ ID NO: 159) |
| 4-7C1 | LC-CDR3 | WQGTHFPLT |
| | | (SEQ ID NO: 159) |
| 4-9H8 | LC-CDR3 | WQGTHFPLT |
| | | (SEQ ID NO: 159) |
| 4-17G9 | LC-CDR3 | WQGTHFPLT |
| | | (SEQ ID NO: 159) |
| 4-18G6 | LC-CDR3 | WQGTHFPLT |
| | | (SEQ ID NO: 159) |
| 4-19A9 | LC-CDR3 | WQGTHFPLT |
| | | (SEQ ID NO: 159) |
| 4-20D2 | LC-CDR3 | WQGTHFPLT |
| | | (SEQ ID NO: 159) |
| Consensus | LC-CDR3 | WQGTHFPLT |
| | | (SEQ ID NO: 159) |

TABLE 14
Consensus Sequences for Group IB
Lambda-5 Antibody VH CDRs

| Antibody | Region | Sequence |
|---|---|---|
| 4-12G1 | HC-CDR1 | DYYLH |
| | | (SEQ ID NO: 79) |
| 4-15E6 | HC-CDR1 | DYYLH |
| | | (SEQ ID NO: 79) |
| Consensus | HC-CDR1 | DYYLH |
| | | (SEQ ID NO: 79) |
| 4-12G1 | HC-CDR2 | WIDPENGATDYAPKFQG |
| | | (SEQ ID NO: 127) |
| 4-15E6 | HC-CDR2 | WIDPENGNTDYAPKFQG |
| | | (SEQ ID NO: 81) |
| Consensus | HC-CDR2 | WIDPENGXTDYAPKFQG |
| | | (SEQ ID NO: 169) |
| | | wherein X at position |
| | | 8 is A or N |
| 4-12G1 | HC-CDR3 | GYYDYDADSAMDY |
| | | (SEQ ID NO: 137) |
| 4-15E6 | HC-CDR3 | GYYDYDTDSAMDY |
| | | (SEQ ID NO: 83) |
| Consensus | HC-CDR3 | GYYDYDXDSAMDY |
| | | (SEQ ID NO: 170) |
| | | wherein X at position |
| | | 7 is A or T |

TABLE 15
Consensus Sequences for Group D3
Lambda-5 Antibody VL CDRs

| Antibody | Region | Sequence |
|---|---|---|
| 4-12G1 | LC-CDR1 | RSSQSLVHSDGITYLH |
| | | (SEQ ID NO: 86) |
| 4-15E6 | LC-CDR1 | RSSQSLVHSDGITYLH |
| | | (SEQ ID NO: 86) |
| Consensus | LC-CDR1 | RSSQSLVHSDGITYLH |
| | | (SEQ ID NO: 86) |
| 4-12G1 | LC-CDR2 | KVSNRFS |
| | | (SEQ ID NO: 88) |
| 4-15E6 | LC-CDR2 | KVSNRFS |
| | | (SEQ ID NO: 88) |
| Consensus | LC-CDR2 | KVSNRFS |
| | | (SEQ ID NO: 88) |
| 4-12G1 | LC-CDR3 | SQSARVPWT |
| | | (SEQ ID NO: 160) |
| 4-15E6 | LC-CDR3 | SQSTRVPWT |
| | | (SEQ ID NO: 90) |
| Consensus | LC-CDR3 | SQTXHVPPT |
| | | (SEQ ID NO: 171) |
| | | wherein X at position |
| | | 4 is A or T |

DNA Encoding Mouse Lambda-5 mAb Heavy and
Light Chain Variable Regions:
SEQ ID NO: 92: DNA encoding 4-15E6 VH
GAGGTGCAGCTGGAGGAGTCTGGGGCAGAGCTTGTGAGGTCAGGG
GCCTCAGTCAAGTTGTCCTGCACAGCTTCTGGCTTCAACATTAAA
GACTACTATTTGCACTGGGTGAAGCAGAGGCCTGAACAGGGCCTG
GAGTGGATTGGATGGATTGATCCTGAGAATGGTAATACTGATTAT
GCCCCGAAGTTCCAGGGCAAGGCCACTATGACTGCAGACACATCC
TCCAACACAGCCTACCTGCAGCTCAGCAGCCTGACATCTGAGGAC
ACTGCCGTCTATTACTGTAATGAGGGGTATTATGATTACGACACA
GACTCTGCTATGGACTACTGGGGTCAAGGAACCTCAGTCACCGTC
TCCTCA
SEQ ID NO: 172: DNA encoding 4-6D12 VH
GAGGTTCAACTCCAGCAGTCTGGGACTGTGCTGGCAAGGCCTGGG
GCTTCCGTGAAGATGTCCTGCAAGGCTTCTGGCTACACCTTTACC
AACTACTGGATGCACTGGGTAAAACAGAGGCCTGGACAGGGTCTA
GAATGGATTGGTGCTATTTATCCTGGAAATAGTGATACTAGCTAC
AACCAGAAGTTCAAGGGCAAGGCCAAACTGACTGCAGTCACATCC
GCCAGCACTGCCTACATGGAGCTCAGCAGCCTGACAAATGAGGAC
TCTGCGGTCTATTTCTGTACAAGGGCTGATTACGACGGGACCCCC
TTTGACTACTGGGGCCAAGGCACCACTCTCACAGTCTCCTCA
SEQ ID NO: 173: DNA encoding 4-5G11 VH
GAGGTTCAGCTCCAGCAGTCTGGGACTGTGCTGGCAAGGCCTGGG
GCTTCCGTGAGGATGTCCTGCAGGGCTTCTGGCTACAGCTTTAAC
AGCTACTGGATGCACTGGGTAAAACAGAGGCCTGGACAGGGTCTA
GAATGGATTGGTGCTATTTATCCTGGAAGTAGTGATACTAGCTAC
AGCCAGAAGTTCAAGGGCAAGGCCAAACTGACTGCAGTCACATCC
GCCAACACTGCCTACATGGAGCTCAGCAGCCTGACAAATGAGGAC
TCTGCGGTCTATTACTGTACAAGGGGTGATTACGACGGGACCCCC
TTTGACTACTGGGGCCAAGGCACCACTCTCACAGTCTCCTCA
SEQ ID NO: 174: DNA encoding 4-7A6 VH
GAGGTTCAACTCCAGCAGTCTGGGACTGTGCTGGCAAGGCCTGGG
ACTTCCGTGAAGATGTCCTGCAAGGCTTCTGGCTACACCTTTACC
AGCTACTGGATGCACTGGGTAAAACAGAGGCCTGGACAGGGTCTA
GAATGGATTGGTGCCATTTATCTTGGAAATACTGATACTAGCTAC
AACCAGAAGTTCAAGGGCAAGGCCAAACTGACTGCAGTCACATCC
GCCAGCAGTGCCTACATGGAGCTCAGCAGCCTGACAAATGAGGAC
TCTGCGGTCTATTATTGTACAAGGGCTGATTACGACGGGACCCCC
TTTGACTACTGGGGCCAAGGCACCACTCTCACAGTCTCCTCA
SEQ ID NO: 175: DNA encoding 4-7C1 VH
GAGGTTCAACTCCAGCAGTCTGGGACTGTGCTGGCAAGGCCTGGG
GCTTCCGTGAAGATGTCCTGCAGGCCTTCTGGCTACACCTTTACC
AGCTACTGGATGCACTGGGTAAAACAGAGGCCTGGACAGGATCTA
GAATGGATTGGTGCTATTTATCCTGGAAATAGTGATACTAGCTAC
AACCAGAAGTTCAAGGGCAAGGCCAAACTGACTGCAGTCACATCC
GCCAGCACTGCCTACATGGAGCTCAGCAGCCTGACAAATGAGGAC
TCTGCGGTCTATTACTGTACAAGGGCTGATTACGACGGGACCCCC
TTTGACTACTGGGGCCAAGGCACCACTCTCACAGTCTCCTCA
SEQ ID NO: 176: DNA encoding 4-9H8 VH
GAGGTTCAGCTCCAGCAGTCTGGGACTGTGCTGGCAAGGCCTGGG
GCTTCCGTGAGGATGTCCTGCAGGGCTTCTGGCTACAGCTTTAAC
AGCTACTGGATGCACTGGGTAAAACAGAGGCCTGGACAGGGTCTA
GAATGGATTGGTGCTATTTATCCTGGAAGTAGTGATACTAGCTAC
AGCCAGAAGTTCAAGGGCAAGGCCAAACTGACTGCAGTCACATCC
GCCAACACTGCCTACATGGAGCTCAGCAGCCTGACAAATGAGGAC
TCTGCGGTCTATTACTGTACAAGGGGTGATTACGACGGGACCCCC
TTTGACTACTGGGGCCAAGGCACCACTCTCACAGTCTCCTCA
SEQ ID NO: 177: DNA encoding 4-12G1 VH
GAGGTGCAGCTGGAGGAGTCTGGGGCAGAGCTTGTGAGGTCAGGG
GCCTCAGTCAAGTTGTCCTGCACAGCTTcTGGCTTCAACATTAAA
GACTACTATTTACACTGGGTGAAGCAGAG
GCCTGAACAGGGCCTGGAGTGGATTGGATGGATTGATCCTGAGAA
TGGTGCCACTGATTATGCCCCGAAGTTCCAGGGCAAGGCCTCTAT
GACTGCAGACACATCCTCCAACACAGCCTACCTGCAGCTCAGCAG
CCTGACATTTGAGGACACTGCCGTCTATTATTGTAATGAGGGGTA
TTATGATTACGACGCGGACTCTGCTATGGACTACTGGGGTCAAGG
AACCTCAGTCACCGTCTCCTCA
SEQ ID NO: 178: DNA encoding 4-17G9 VH
GAGGTTCAACTCCAGCAGTCTGGGACTGTGCTGGCAAGGCCTGGG
GCTTCCGTGAAGATGTCCTGCCAGGCTTCTGGCTACACCTTTACC
AACTACTGGATGCACTGGGTAAAACAGAGGCCTGGACAGGGTCTA
GAATGGATTGGTGCTATCTATCCTGGAAATAGTGATACTAGCTAT
AACCAGAATTTCAAGGGCAAGGCCGAACTGACTGCAGTCACATCC
GCCACCACTGCCTACATGGAACTCAGCAGCCTGACAGATGAAGAC
TCTGCGGTCTATTACTGTACAAGGGCTGATTACGACGGGACCCCC
TTTGACTACTGGGGCCAAGGCACCACTCTCACAGTCTCCTCA
SEQ ID NO: 179: DNA encoding 4-18G6 VH
GAGGTTCAACTCCAGCAGTCTGGGACTGTGCTGGCAAGGCCTGGG
GCTTCCGTGAAGATGTCCTGCAAGGCTTCTGGCTACACCTTTACC
AACTACTGGATGCACTGGGTAAAACAGAGGCCTGGACAGGGTCTA
GAATGGATTGGTGCTGTTTATCCTGGAAACAGTGATACTAGTTAC
AGCCAGAAGTTCACGGGCAAGGCCAAACTGACTGCAGTCACATCC
GCCAGCACTGCCTACATGGACCTCAGCAGCCTGACAAATGAGGAC
TCTGCGGTCTATTACTGTACAAGGGCTGATTACGACGGGACCCCC
TTTGACTACTGGGGCCAAGGCACTACTCTCACAGTCTCCTCA
SEQ ID NO: 180: DNA encoding 4-19A9 VH
GAGGTTCAACTCCAGCAGTCTGGGACTGTGCTGGCAAGGCCTGGG
GCTTCCGTGAAGATGTCCTGCAAGGCTTCTGGCTACACCTTTACC
AGCTACTGGATGCACTGGGTAAAACAGAGGCCTGGACAGGGTCTA
GAATGGATTGGTGCTATTTATCCTGGAAATAGTGATACTAGCTAC
AACCAGAAGTTCAAGGGCAAGGCCAGACTGACTGCAGTCACATCC
GCCAGCACTGCCTACATGGAGCTCAGCAGCCTGACAAATGAGGAC
TCTGCGGTCTATTATTGTACAAGGGCTGATTACGACGGGACCCCC
TTTGACTACTGGGGCCAAGGCACCACTCTCACAGTCTCCTCA
SEQ ID NO: 181: DNA encoding 4-20D2 VH
GAGGTTCAGCTCCAGCAGTCTGGGACTGTGCTGGCAAGGCCTGGG
GCTTCCGTGAAGATGTCCTGCAAGGCTTCTGGCTACAGCTTTACC
AGCTACTGGATGCACTGGGTAAAACAGAGGCCTGGACAGGGTCTA
GAATGGATTGGTGCTATTTATCCTGGAAATAGTGATACTAGCTAC
AACCAGAAGTTCAAGGGCAAGGCCAAACTGACTGCAGTCACATCC
GCCAGCACTGCCTACATGGAGCTCAGCAGCCTGACAAATGAGGAC
TCTGCGGTCTATTACTGTACAAGGGGTGATTACGACGGGACCCCC
TTTGACTACTGGGGCCAAGGCACCACTCTCACAGTCTCCTCA
SEQ ID NO: 93: DNA encoding 4-15E6 VL
GATGTTTTGATGACCCAAACTCCACTCTCCCTGCCTGTCAGTCTT
GGAGATCAAGCCTCCATCTCTTGCAGATCTAGTCAGAGCCTTGTA
CACAGTGATGGAATCACCTATTTACATTGGTACCTACAGAAGCCA
GGCCAGTCTCCAAAACTCCTGATCTACAAAGTTTCCAACCGATTT
TCTGGGGTCCCAGACAGGTTCAGTGGCAGTGGATCAGGGACAGAT
TTCACACTCAAGATCAGCAGAGTGGAGGCTGAGGATCTGGGAGTT
TATTTCTGCTCTCAAAGTACACGTGTTCCGTGGACGTTCGGTGGA
GGCACCAAGCTGGAAATCAAACGG
SEQ ID NO: 182: DNA encoding 4-6D12 VL
GACATTCTGATGACCCAGTCTCCACTCACTTTGTCGGTTACCATT
GGACACCCAGCCTCCATCTCTTGCAAGTCAAGTCAGAGCCTCTTA
GATAGTGATGGAGAGACATATTTGAGTTGGTTGTTACAGAGGCCA
GGCCAGTCTCCAGAGCGCCTAATCTATCTGGTGTCTAAACTGGAC
TCTGGAGTCCCTGACAGGTTCACTGGCAGTGGATCAGGGACAGAT
TTCACACTGAAAATCAGCAGAGTGGAGGCTGAGGATTTGGGAGTT
TATTATTGTTGGCAAGGTACACATTTTCCGCTCACGTTCGGTGCT
GGGACCAAGCTGGAGCTGAAACGG
SEQ ID NO: 183: DNA encoding 4-5G11 VL
GACATTCTGATGACCCAGTCTCCACTCACTTTGTCGGTTACCATT
GGACAACCAGCCTCCATCTCTTGCAAGTCAGGTCAGAGCCTCTTA
GATAGTGATGGAAAGACATATTTGAATTGGTTGTTACAGAGGCCA
GGCCAGTCTCCAAAGCGCCTAATCTATCTGGTGTCTAAACTGCAC
TCTGGAGTCCCTGACAGGTTCACTGGCAGTGGATCAGGGACAGAT
TTCACACTGAAAATCAGCAGAGTGGAGGCTGAGGATTTGGGAGTT
TATTATTGCTGGCAAGGTACACATTTTCCGCTCACGTTCGGTGCT
GGGACCAAGCTGGAGCTGAAACGG
SEQ ID NO: 184: DNA encoding 4-7A6 VL
GATGTTGTGATGACCCAGAATGCACTCACTTTGTCGGTTACCATC
GGACACCCAGCCTCCATCTCTTGTAGGTCAAGTCAGAGCCTCTTA
GATAGTGATGGAGAGACATATTTGAGTTGGTTGTTACAGAGGCCA
GGCCAGTCTCCAAAGCGCCTAATCTATCTGGTGTCTAAATTGGAC
TCTGGAGTCCCTGACAGGTTCACTGGCAGTGGATCAGGGACAGAT
TTCACACTGAAAATCAGCAGAGTGGAGGCTGAGGATTTGGGAGTT
TATTATTGTTGGCAAGGCACACATTTTCCGCTCACGTTCGGTGCT
GGGACCAAGCTGGAGCTGAAACGG
SEQ ID NO: 185: DNA encoding 4-7C1 VL
GACATTGTGATGACCCAGTCTCCACTCACTTTGTCGGTTACCATT
GGACACCCAGCCTCCATCTCTTGCAAGTCAAGTCAGAGCCTCTTA
GATAGTGATGGAGAGACATATTTGAGTTGGTTGTTACAGAGGCCA
GGCCAGTCTCCAAAGCGCCTAATCTATCTGGTGTCTAAACTGGAC
TCTGGAGTCCCTGACAGGTTCACTGGCAGTGGATCAGGGACAGAT
TTCACACTGAAAATCAGCAGAGTGGAGGCTGAGGATTTGGGAGTT
TATTATTGTTGGCAAGGTACACATTTTCCGCTCACGTTCGGTGCT
GGGACCAAGCTGGAGCTGAAACGG
SEQ ID NO: 186: DNA encoding 4-9H8 VL
GACATTGTGATGACCCAGTCTCCACTCACTTTGTCGGTTACCATT
GGACAACCAGCCTCCATCTCTTGCAAGTCAGGTCAGAGCCTCTTA
GATAGTGATGGAAAGACATATTTGAATTGGTTGTTACAGAGGCCA
GGCCAGTCTCCAAAGCGCCTAATCTATCTGGTGTCTAAACTGCAC
TCTGGAGTCCCTGACAGGTTCACTGGCAGTGGATCAGGGACAGAT
TTCACACTGAAAATCAGCAGAGTGGAGGCTGAGGATTTGGGAGTT
TATTATTGCTGGCAAGGTACACATTTTCCGCTCACGTTCGGTGCT
GGGACCAAGCTGGAGCTGAAACGG
SEQ ID NO: 187: DNA encoding 4-12G1 VL
GATGTTTTGATGACCCAAACTCCACTCTCCCTGCCTGTCAGTCTT
GGAGATCAAGCCTCCATCTCTTGCAGATCTAGTCAGAGCCTTGTA
CACAGTGATGGAATCACCTATTTACATTGGTACCTGCAGAAGCCA
GGCCAGTCTCCAAAGCTCCTGATCTACAAAGTTTCCAACCGATTT
TCTGGGGTCCCAGACAGGTTCAGTGGCAGTGGATCAGGGACAGAT
TTCACACTCAAGATCAGCAGAGTGGAGGCTGAGGATCTGGGAGTT
TATTTCTGCTCTCAAAGTGCACGTGTTCCGTGGACATTCGGTGGA
GGCACCAAGCTGGAAATCAAACGG
SEQ ID NO: 188: DNA encoding 4-17G9 VL
GACATTGTGATGACCCAGTCTCCACTCACTTTGTCGGTTACCATT
GGACACCCAGCCTCCATCTCTTGCAAGTCAAGTCAGAGCCTCTTA
GATAGTGATGGAGAGACATATTTGAGTTGGTTGTTACAGAGGCCA
GGCCAGTCTCCAAAGCGCCTAATCTATCTGGTGTCTAAACTGGAC
TCTGGAGTCCCTGACAGGTTCACTGGCAGTGGATCAGGGACAGAT
TTCACACTGAAAATCAGCAGAGTGGAGGCTGAGGATTTGGGAGTT
TATTATTGTTGGCAAGGTACACATTTTCCGCTCACGTTCGGTGCT
GGGACCAAGGTGGAGCTGAAACGG
SEQ ID NO: 189: DNA encoding 4-18G6 VL
GATGTTTTGATGACCCAAACTCCACTCACTTTGTCGGTTATCATT
GGACAGCCAGCCTCCATCTCTTGCAAGTCAAGTCAGAGCCTCTTA
GATAGTGATGGAGAGACATATTTGAGTTGGTTGTTACAGAGGCCA
GGCCAGTCTCCAAAGCGCCTAATCTATCTGGTGTCTAAACTGGAC
TCTGGAGTCCCTGACCGGTTCACTGGCAGTGGATCAGGGACAGAT
TTCACACTGAAAATCAGCAGAGTGGAGGCTGAGGATTTGGGAGTT
TATTATTGTTGGCAAGGTACACATTTTCCGCTCACGTTCGGTGCT
GGGACCAAGCTGGAGCTGAAACGG
SEQ ID NO: 190: DNA encoding 4-19A9 VL
GACATTGTGATGACCCAGTCTCCACTCACTTTGTCGGTTACCATT
GGACACCCAGCCTCCATCTCTTGCAAGTCAAGTCAGAGCCTCTTA
GATAGTGATGGAGAGACATATTTGAGTTGGTTGTTACAGAGGCCA
GGCCAGTCTCCAAAGCGCCTAATCTATCTGGTGTCTAAACTGGAC
TCTGGAGTCCCTGACAGGTTCACTGGCAGTGGATCAGGGACAGAT
TTCACACTGAAAATCAGCAGAGTGGAGGCTGAGGATTTGGGAGTT
TATTATTGTTGGCAAGGTACACATTTTCCGCTCACGTTCGGTGCT
GGGACCAAGCTGGAGCTGAAACGG
SEQ ID NO: 191: DNA encoding 4-20D2 VL
GATGTTTTGATGACCCAAACTCCACTCACTTTGTCGGTTACCATT
GGACAACCAGCCTCCATCTCTTGCAAGTCAAGTCAGAGCCTCTTA
GATAGTGATGGAGAGACATATTTGAATTGGTTGTTACAGAGGCCA
GGCCAGTCTCCAAAGCGCCTAATCTATCTGGCGTCTAAACTGGAC
TCTGGAGTCCCTGACAGGTTCACTGGCAGTGGATCAGGGACAGAT
TTCACACTGAAAATCAGCAGAGTGGAGGCTGAGGATTTGGGAATT
TATTATTGCTGGCAAGGTACACATTTTCCGCTCACGTTCGGTGCT
GGGACCAAGCTGGAGCTGAAACGG

### Example 4. Binding Characteristics of Pre-BCR Antibodies (VpreB)

VpreB mAbs purified from hybridoma supernatants or recombinant, chimeric VpreB mAbs described in Example 3 were evaluated by flow cytometry for binding to the pre-BCR-expressing pre-B cell line, NALM-6, and for the absence of binding to the VpreB-null B cell lines Ramos and Raji. Binding to additional cell lines representing colorectal cancer (COLO 205), T cell leukemia (Jurkat), and mouse pre-B cell (L1.2) was also evaluated. Cells were incubated with 20 nM of each antibody, which was detected using PE/Cy5-labeled secondary antibodies: mouse anti-human IgG1 antibody (Southern Biotech #9042-13) for chimeric antibodies or goat anti-mouse F(ab′)₂ IgG (Southern Biotech #1032-13) for purified hybridoma antibodies.

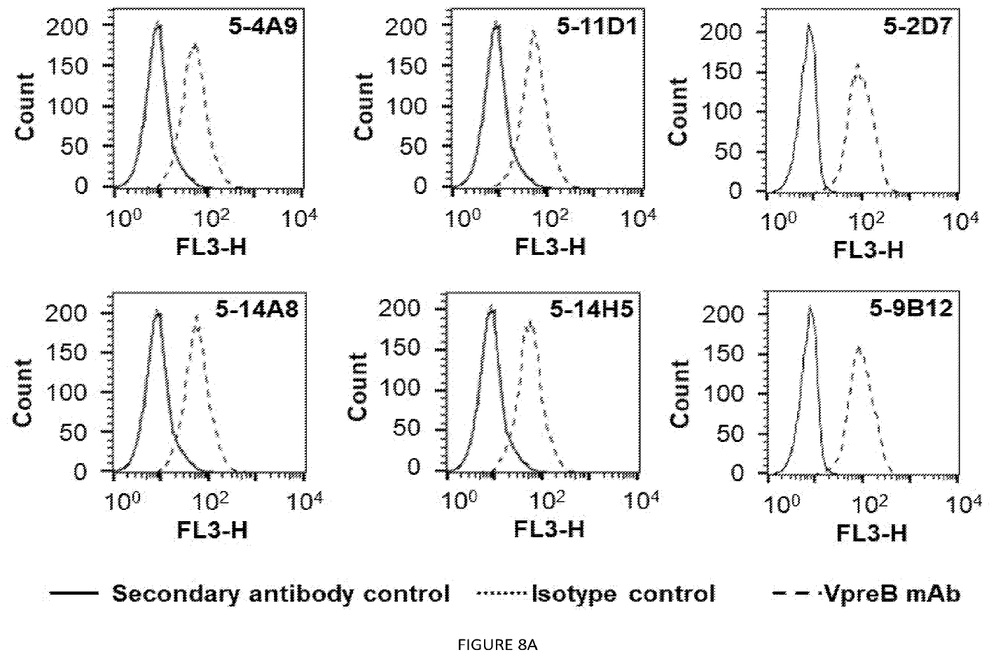

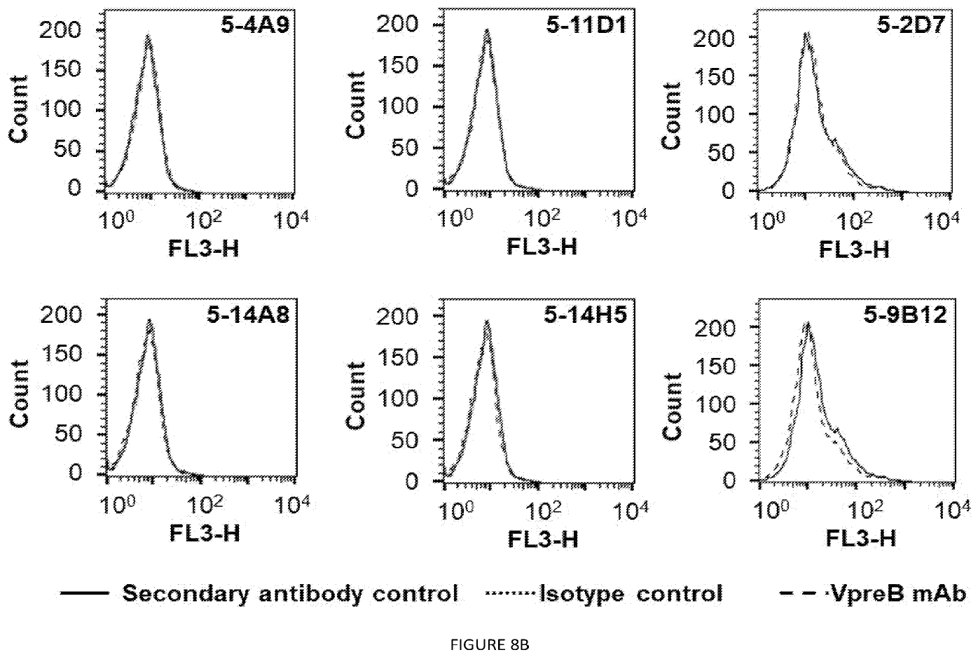

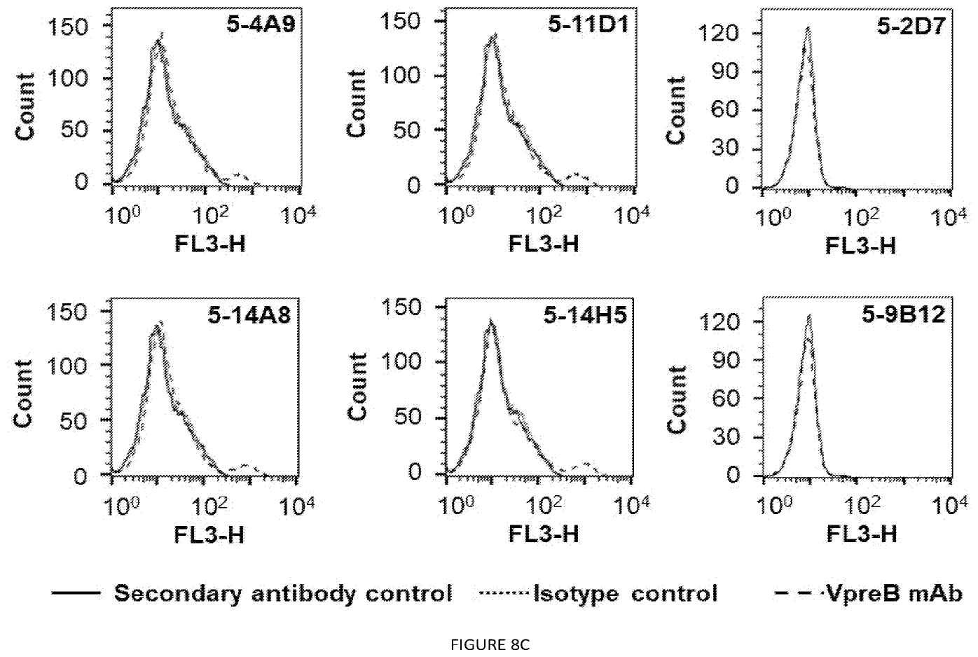

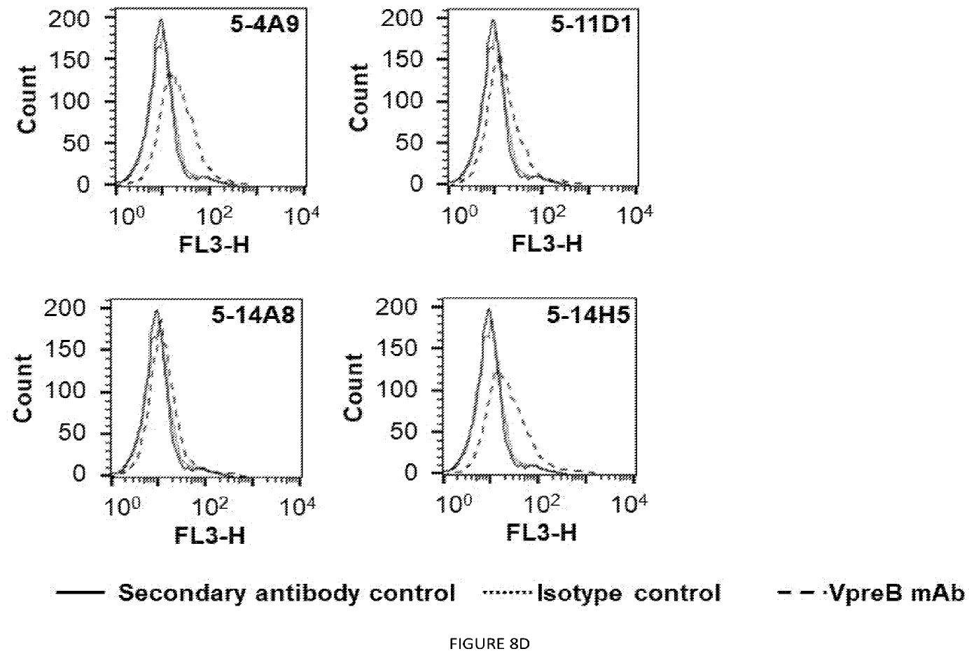

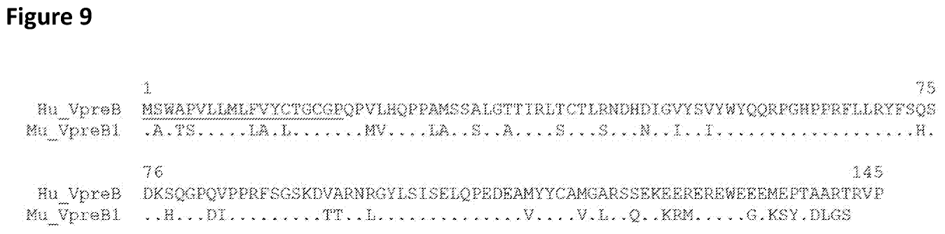

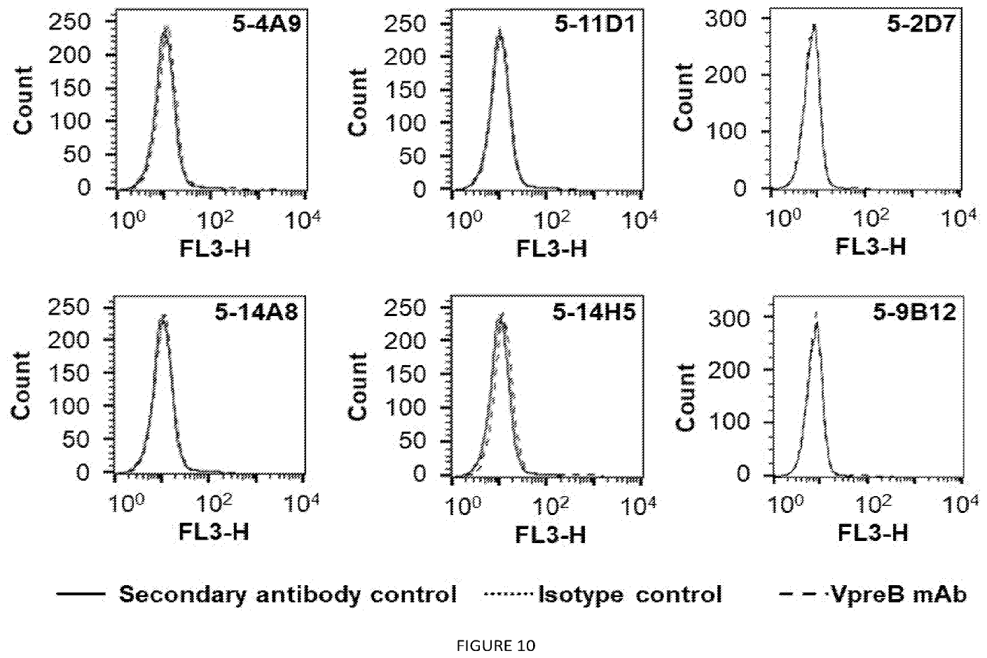

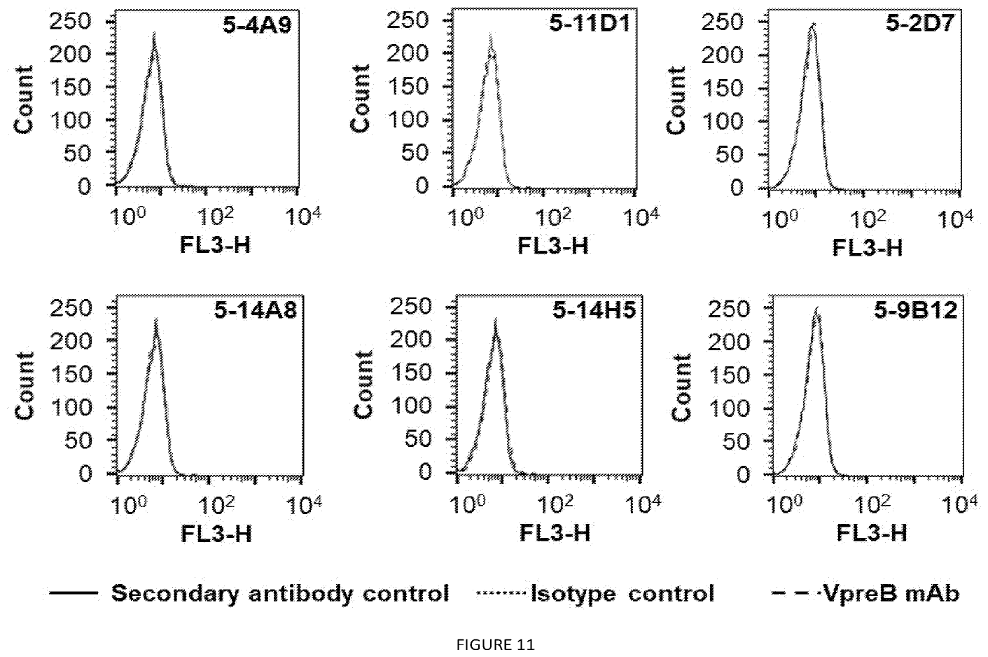

VpreB antibodies 5-2D7, 5-4A9, 5-9B12, 5-11D1, 5-14A8, and 5-14H5 all bound NALM-6 but not Ramos, Raji, COLO 205, or Jurkat, demonstrating pre-BCR selectivity (FIGS. 8A-8D; 10, 11). Three of the antibodies, 5-4A9, 5-11D1, and 5-14H5, showed modest cross-reactivity with the mouse pre-B cell line, L1.2 (FIG. 8D). This is a surprising outcome, as the human and mouse VpreB protein sequences are 72.5% identical (FIG. 9) and the antibodies were generated in the mouse, which would be expected to have immunological tolerance for the protein.

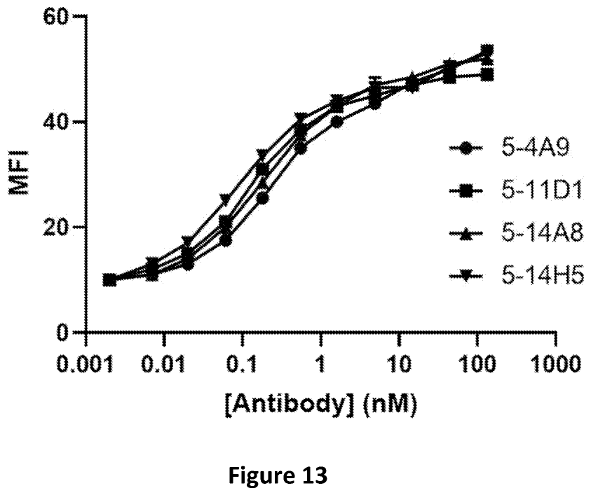

Avidity of each VpreB antibody was determined by measuring saturation binding kinetics of antibody to NALM-6 cells using flow cytometry as follows. The cells were stained with a 3-fold descending titration of recombinant, chimeric VpreB antibody ranging from 133 nM to 0.002 nM. A PE/Cy5-labeled mouse anti-human IgG1 secondary antibody (Southern Biotech #9042-13) was used to detect cell-bound anti-VpreB or an IgG1 isotype control antibody (PAS-ISO6H4). The 50% effective concentration (EC₅₀) was calculated using GraphPad Prism software. Antibodies 5-4A9, 5-11D1, 5-14A8, and 5-14H5 all bound to VpreB-expressing NALM-6 cells with unusually strong binding avidity (<400 pM; FIG. 13, Table 16).

TABLE 16
Effective concentrations at 50%
(EC₅₀) of maximum binding of
VpreB antibodies to NALM-6 cells.

| Antibody | EC₅₀ (nM) |
|---|---|
| 5-4A9 | 0.396 |
| 5-11D1 | 0.143 |
| 5-14A8 | 0.227 |
| 5-14H5 | 0.115 |

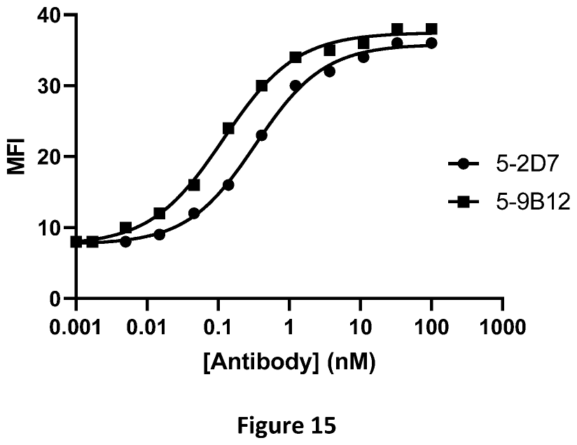

The binding avidity of the VpreB mAbs 5-2D7 and 5-9B12 was also determined by measuring saturation binding kinetics of recombinant, chimeric mAb to NALM-6 cells using flow cytometry as described above. Both mAbs bound to VpreB-expressing NALM-6 cells with unusually strong avidity (<400 pM; FIG. 15, Table 17).

TABLE 17
Effective concentrations at 50%
(EC₅₀) of maximum binding of VpreB
antibodies to NALM-6 cells.

| Antibody | EC₅₀ (nM) |
|---|---|
| 5-2D7 | 0.34 |
| 5-9B12 | 0.12 |

### Example 5. Binding Characteristics of Pre-BCR Antibodies (Lambda-5)

Lambda-5 mAbs purified from hybridoma supernatants or recombinant, chimeric lambda-5 mAbs described in Example 3 were evaluated by flow cytometry for binding to the pre-BCR-expressing pre-B cell line, NALM-6, and for the absence of binding to the VpreB-null B cell lines Ramos and Raji. Binding to additional cell lines was also evaluated. Cells were incubated with 20 nM of each antibody, which was detected using PE/Cy5-labeled secondary antibodies: mouse anti-human IgG1 antibody (Southern Biotech #9042-13) for chimeric antibodies or goat anti-mouse F(ab′)₂ IgG (Southern Biotech #1032-13) for purified hybridoma antibodies.

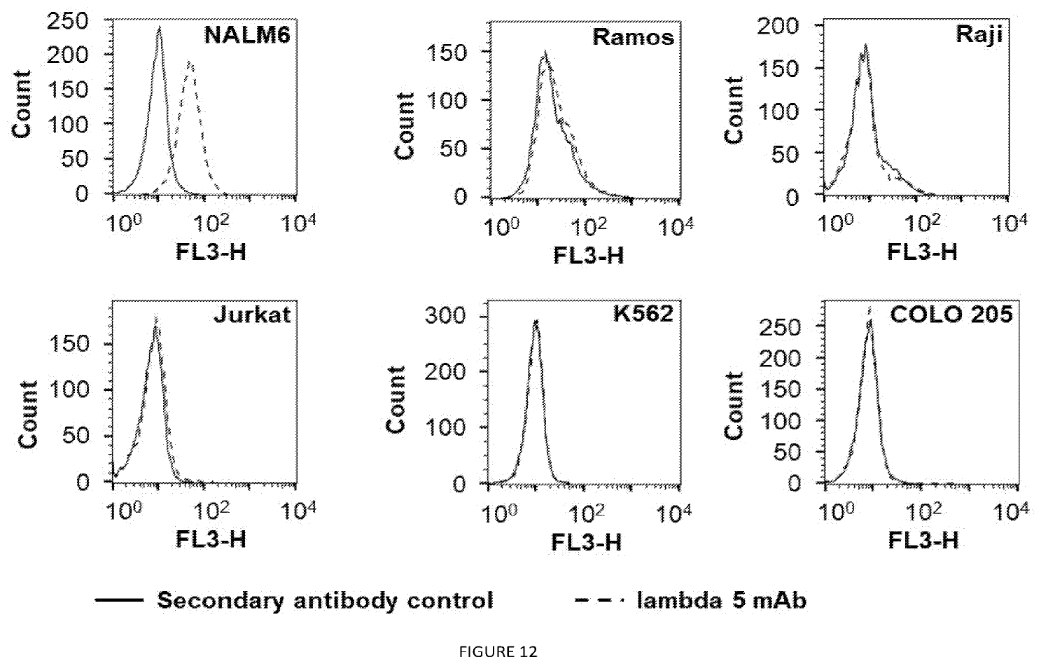

The recombinant, chimeric lambda-5 antibody 4-15E6 bound the human pre-B cell line NALM-6 but not the B cell lines Ramos and Raji, demonstrating pre-BCR selectivity (FIG. 12). The antibody did not bind Jurkat, COLO 205, or the human erythroleukemia cell line K562.

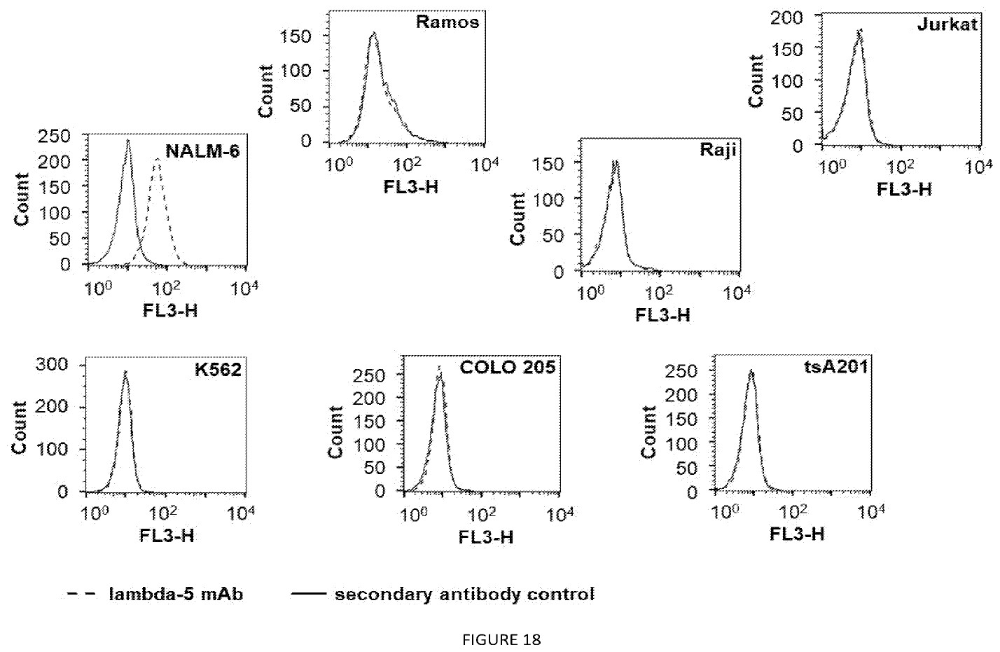

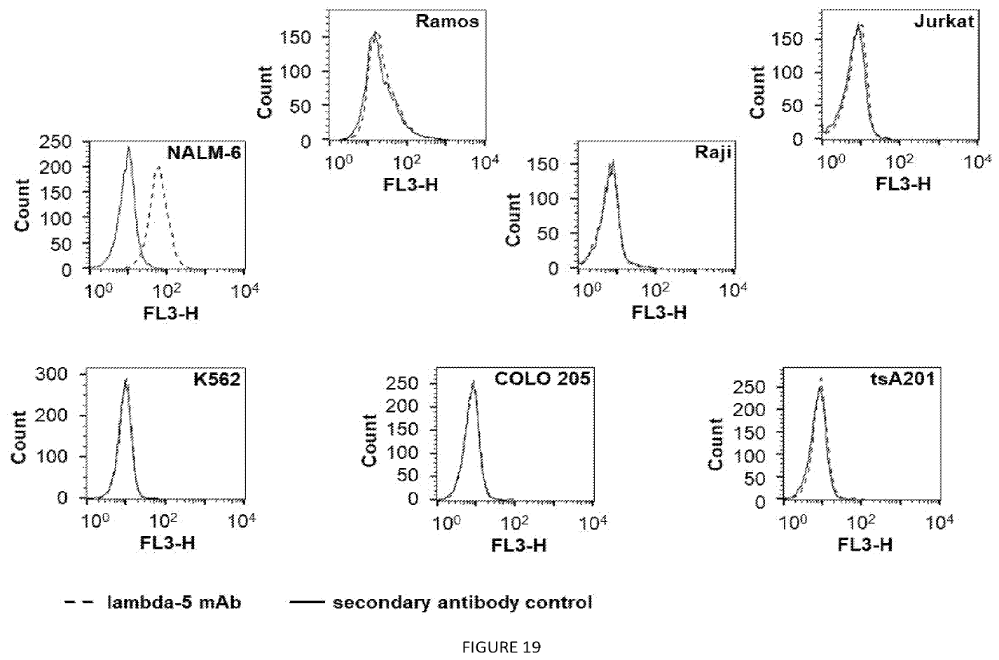

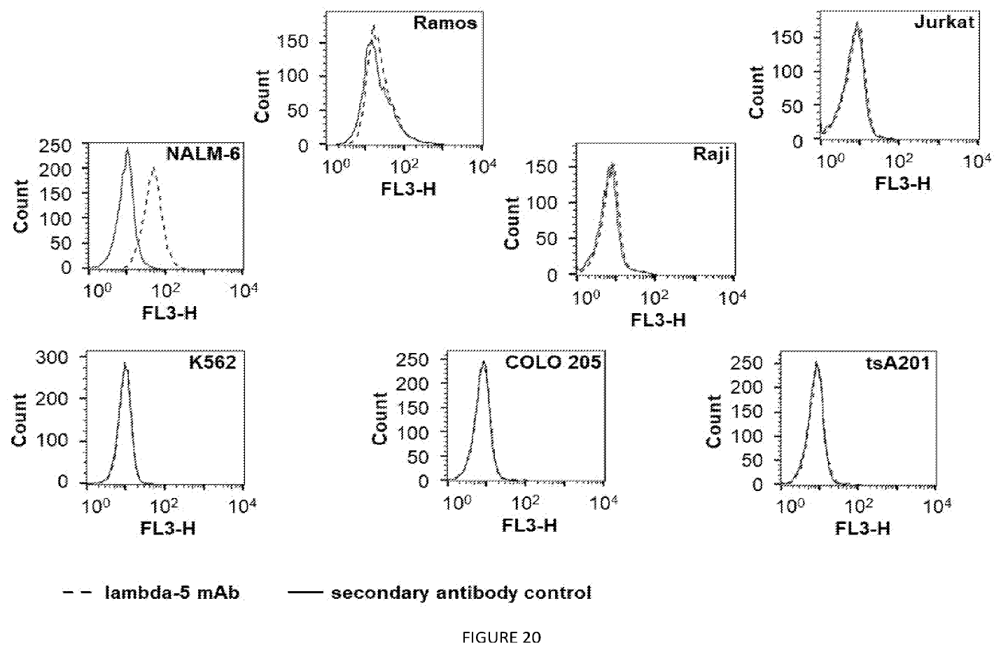

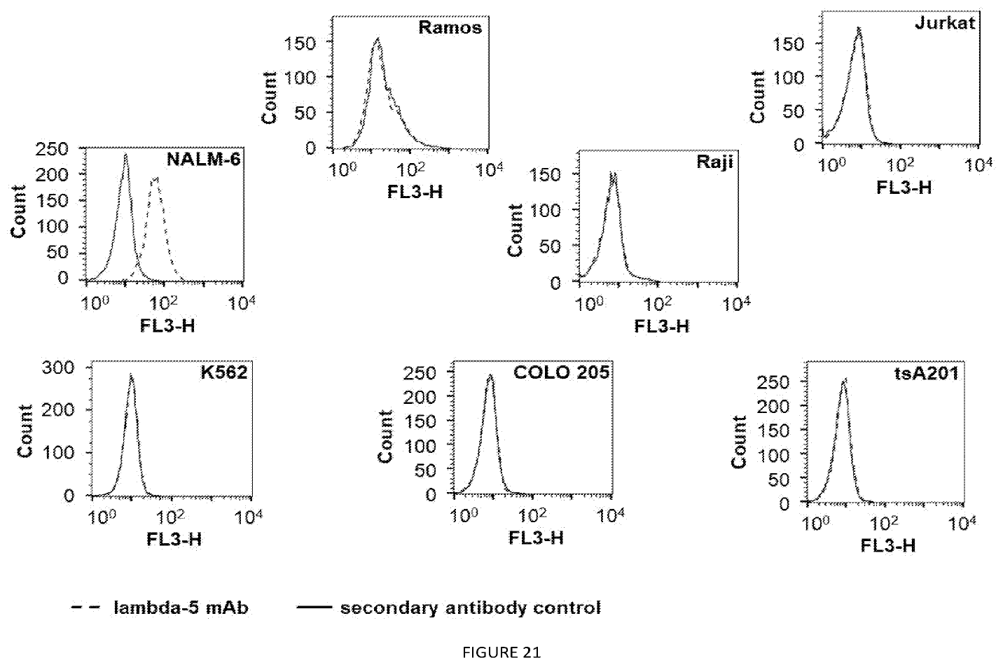

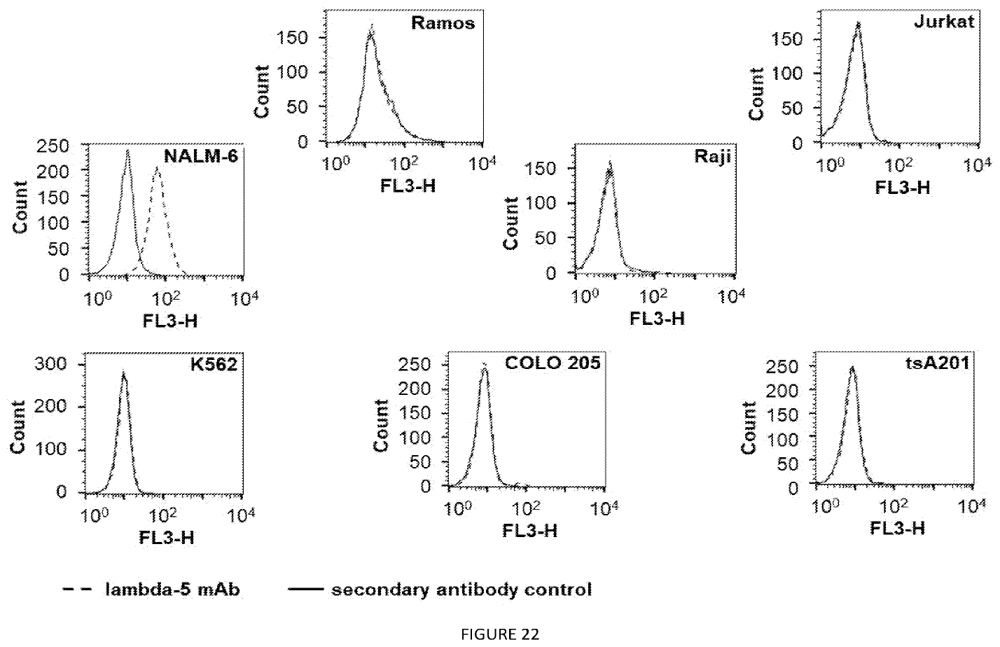

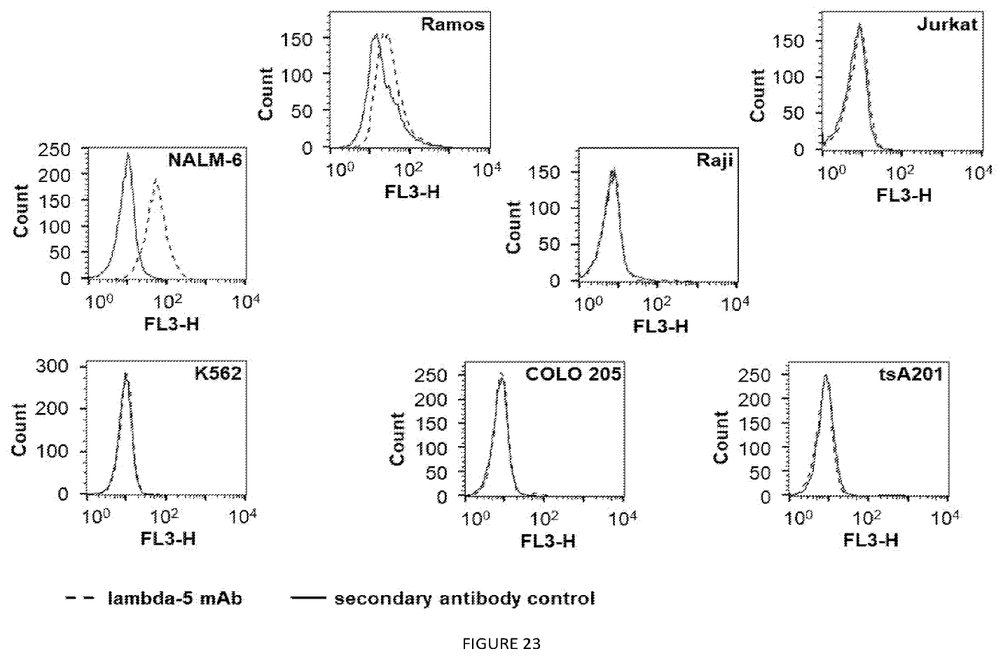

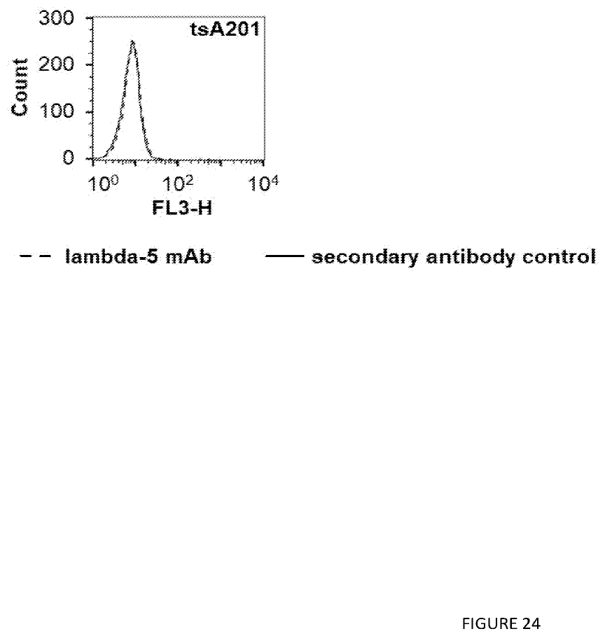

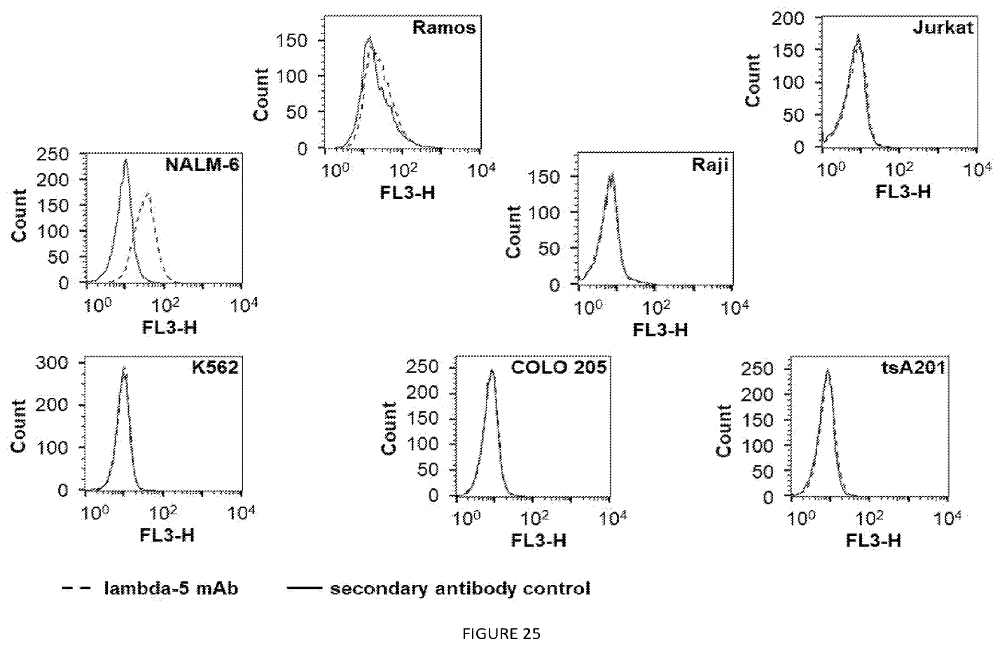

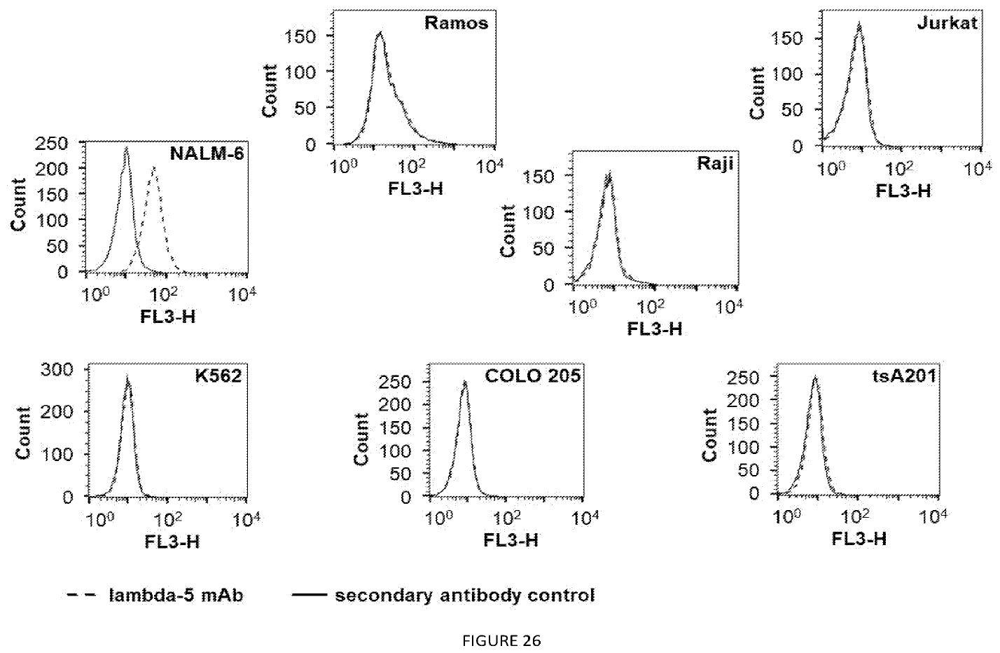

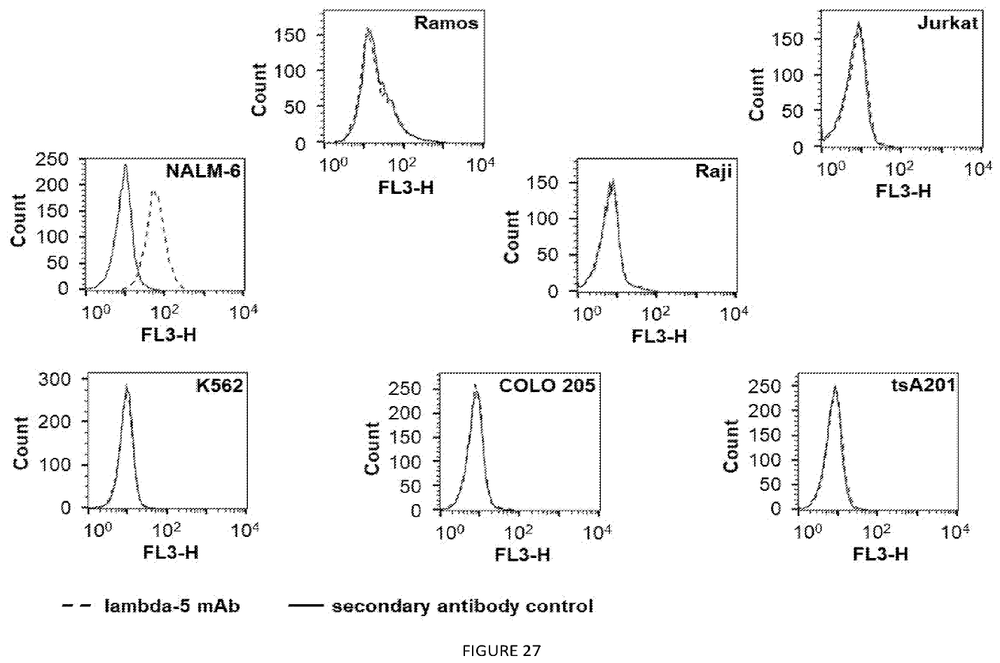

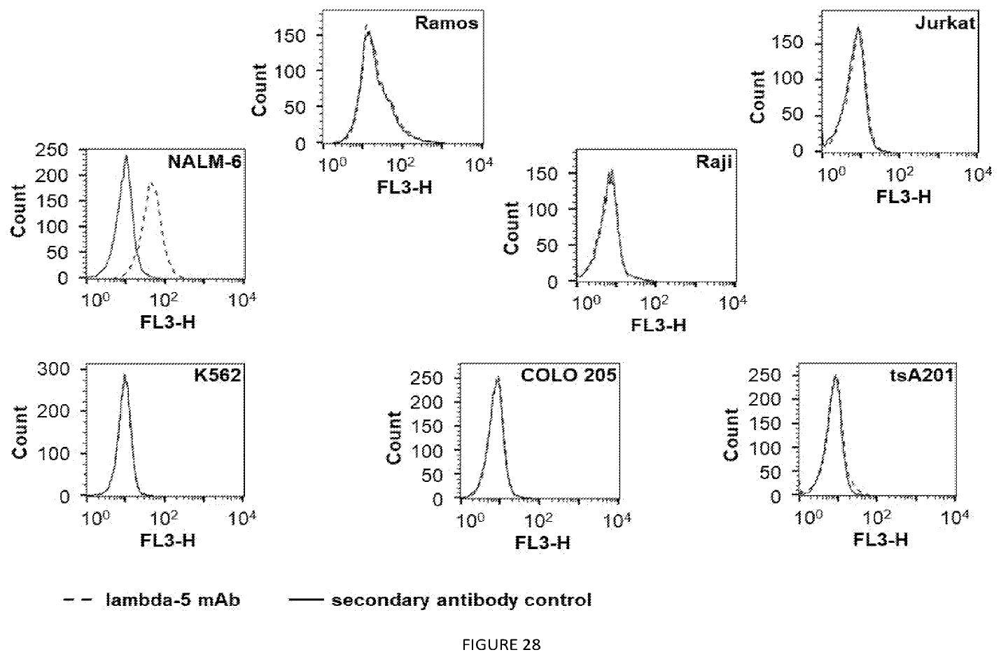

The additional lambda-5 antibodies 4-6D12, 4-5G11, 4-7A6, 4-7C1, 4-9H8, 4-12G1, 4-17G9, 4-18G6, 4-19A9, and 4-20D2 were purified from their corresponding hybridomas using Protein G. The mAbs were evaluated by flow cytometry for binding to the pre-BCR-expressing pre-B cell line, NALM-6, and for the absence of binding to the VpreB-null B cell lines Ramos and Raji. Binding to additional cell lines representing colorectal cancer (COLO 205), T cell leukemia (Jurkat), erythroleukemia (K562), and human embryonic kidney (tsA201) were also evaluated. The results are illustrated in FIGS. 18-23 and 25-28. All of the mAbs, with the possible exception of 4-12G1, bound only NALM-6 cells, demonstrating their specificity for the pre-BCR. The 4-21G1 mAb showed some binding to Ramos cells (FIG. 23), suggesting cross-reactivity with an antigen on the cell line. The recombinant, chimeric 4-15E8 mAb, previously evaluated for binding to the NALM-6, Ramos, Raji, Jurkat, COLO 205, and K562 cell lines (FIG. 12), was further tested and found to not bind to tsA201, as illustrated in FIG. 24.

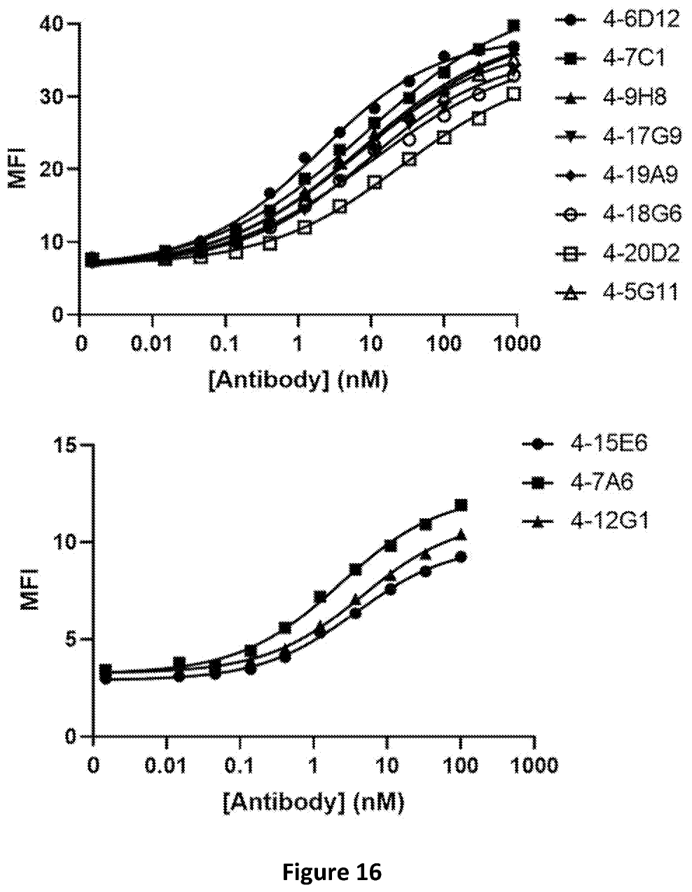

The avidity of each lambda-5 antibody was determined by measuring saturation binding kinetics of recombinant, chimeric mAb to NALM-6 cells using flow cytometry as described for the VpreB antibody saturation binding kinetics experiment. Antibodies 4-6D12, 4-5G11, 4-7A6, 4-7C1, 4-9H8, 4-12G1, 4-15E6, 4-17G9, 4-18G6, 4-19A9, and 4-20D2 all bound to VpreB- expressing NALM-6 cells with strong avidity (<30 nM; FIG. 16, Table 18).

TABLE 18
Effective concentrations at 50%
(EC₅₀) of maximum binding of
lambda-5 antibodies to NALM-6 cells.

| Antibody | EC₅₀ (nM) |
|---|---|
| 4-6D12 | 1.9 |
| 4-5G11 | 6.2 |
| 4-7A6 | 2.3 |
| 4-7C1 | 8.4 |
| 4-9H8 | 7.4 |
| 4-12G1 | 4.5 |
| 4-15E6 | 3.7 |
| 4-17G9 | 9.0 |
| 4-18G6 | 10.2 |
| 4-19A9 | 8.1 |
| 4-20D2 | 26.3 |

### Example 6. Competition Binding of Pre-BCR Antibodies

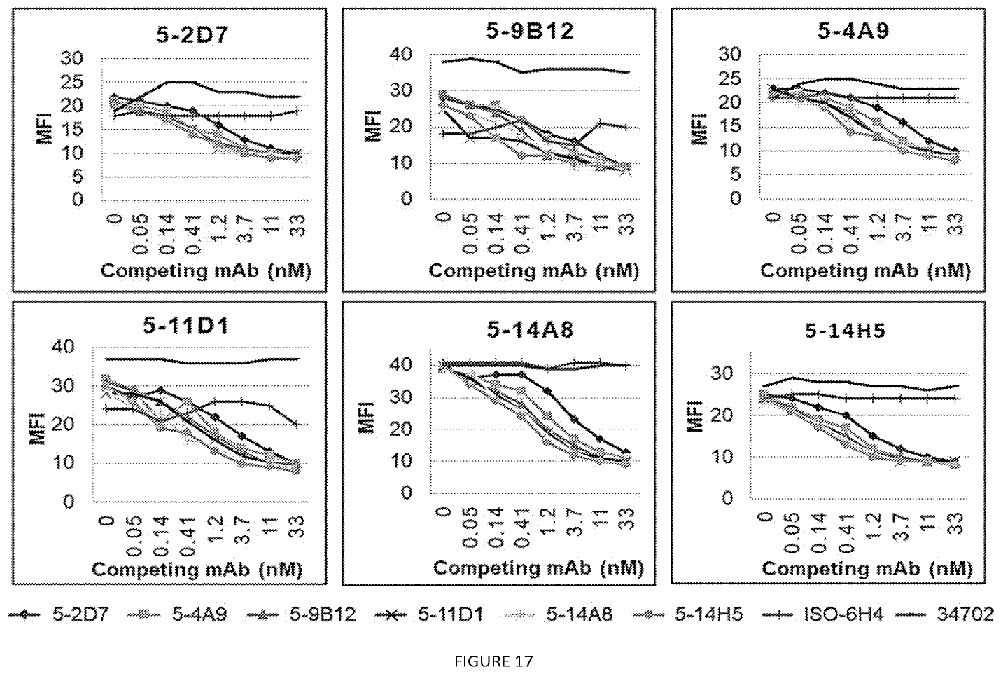

The VpreB mAbs were tested in a competition binding experiment to determine the number of potential distinct epitopes bound by the antibodies. Each of the six VpreB mAbs was biotinylated (Thermo Scientific, #A39257) according to the manufacturer's protocol. A preliminary binding experiment revealed that biotinylation slightly reduced the EC₅₀ of some of the mAbs on NALM-6 cells. These results were used to identify a concentration between the EC₅₀ and maximum binding of each biotinylated mAb to be used for the competition experiment. Accordingly, the biotinylated mAbs were tested at the following concentrations: 33.3 nM (5-2D7, 5-4A9), 1.23 nM (5-14H5), or 1.0 nM (5-9B12, 5-11D1, 5-14A8). NALM-6 cells were seeded at a density of 6×10⁴ cells/well in a 96-well culture plate and incubated at 37° C., 5% CO₂ for 24 h. A 12-point, 3-fold stepwise titration from 100 nM to 0.001 nM of each unlabeled mAb was then added in triplicate wells along with each biotinylated mAb to the NALM-6 cells. After one hour of incubation on ice, bound biotinylated mAb was exposed using streptavidin-SPRD (Southern Biotech, #7100-13S) and detected by flow cytometry. As illustrated in FIG. 17, addition of increasing concentrations of a non-labeled VpreB mAb to each of the biotinylated VpreB mAbs results in decreasing binding of the biotinylated mAb to the NALM-6 cells. This outcome indicates that mAbs 5-4A9, 5-2D7, 5-9B12, 5-11D1, 5-14H5 and 5-14A8 all compete with each other for binding to VpreB on NALM-6 cells, indicating that they all bind to a single epitope or to overlapping linear or conformational epitopes. In contrast, a commercially available control VpreB mAb (Southern Biotech #347402) did not compete for binding with the six test mAbs, indicating that it binds a distinct epitope. An isotype control mAb, ISO6H4, designed and produced in the same manner as the six VpreB mAbs, also did not compete for binding with the VpreB mAbs, ruling out any contribution of the non-variable components of the antibodies to the competitive binding.

### Example 7. Internalization of VpreB Antibodies into a Leukemia Cell Line

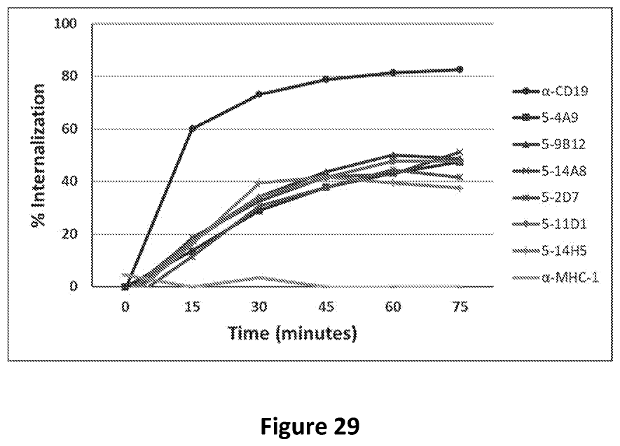

As discussed above, cross-linking the pre-BCR results in its internalization into the cell. The six VpreB mAbs were tested for internalization into NALM-6 cells. The cells were incubated at 37° C. or 4° C., 5% CO₂ with 67 nM of each mAb for various times, washed with buffer, then remaining cell surface mAb was detected by flow cytometry using a mouse anti-human IgG secondary mAb (Southern Biotech #9042-13). MAbs against CD19 (Southern Biotech #9340-01) and MHC-I (ThermoFisher Scientific #14-9983-82), both of which are expressed by NALM-6, were included as positive and negative controls, respectively. Percent internalization was calculated as 100-((MFI37° C./MFI4° C.)*100). 40-50% of each VpreB mAb was internalized within an hour (FIG. 29). All of the VpreB mAbs were internalized at similar rates, with maximum internalization reached by 60 min. The incomplete VpreB mAb internalization relative to that of the CD19 positive control mAb may reflect the different densities of the target molecules on NALM-6 cells. There are 10,000 molecules of VpreB (Erasmus et al., Science Signaling 9:ra116, 2016) but up to 56,000 molecules of CD19 per cell (Gerber et al., Blood 113:4352, 2009; Haso et al., Blood 121:1165, 2013). The negative control molecule, MHC-I, is not internalized.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters herein are approximations that may vary depending upon the standard deviation found in their respective testing measurements.

The above examples are offered by way of illustration, and not limitation. It will be apparent to those of skill in the art that variations may be applied to the articles and methods without departing from the spirit and scope of the disclosure. All such variations and equivalents apparent to those skilled in the art, whether now existing or later developed, are deemed to be within the spirit and scope of the disclosure as defined by the appended claims. All patents, patent applications, and publications mentioned in the specification are indicative of the levels of those of ordinary skill in the art to which the disclosure pertains. All patents, patent applications, and publications are herein incorporated by reference in their entirety for all purposes and to the same extent as if each individual publication was specifically and individually indicated to be incorporated by reference in its entirety for any and all purposes. The disclosure illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this disclosure as defined by the appended claims.

What is claimed is:

1. An isolated antibody specific for pre-BCR, or antigen-binding fragment thereof, that specifically binds to human pre-BCR, optionally as part of a sterile composition comprising pharmaceutically acceptable excipients.

2. The antibody, or antigen-binding fragment of claim 1 wherein the antibody or antigen-binding fragment thereof is a human VpreB- or human lambda-5-specific antibody.

3. An isolated antibody, or antigen-binding fragment that specifically binds to the VpreB subunit of the SLC of human pre-BCR that comprises:
a. a VH comprising a HC CDR1 set forth as SEQ ID NO:30 (SDYWT); a HC CDR2 SEQ ID NO:32 (YISYSGRTYYNPSLKS); and a HC CDR3 SEQ ID NO:34 (ERYYYGSLDY); and/or a VL comprising a LC CDR1 set forth as SEQ ID NO:48 (RSSQSLVHSNGNTYLH); a LC CDR2 set forth as SEQ ID NO:44 (KVSNRFS); and a LC CDR3 set forth as SEQ ID NO:53 (SQTTHVPPT) [mAb 5-11D1]; or
b. a VH comprising a HC CDR1 set forth as SEQ ID NO:19 (SYWMQ); a HC CDR2 SEQ ID NO:21 (EINPSNGRINYNEKFKS); and a HC CDR3 SEQ ID NO:23 (SGLLDY); and/or a VL comprising a LC CDR1 set forth as SEQ ID NO:42 (RSSQSLIHSNGNTYLH); a LC CDR2 set forth as SEQ ID NO:44 (KVSNRFS); and a LC CDR3 set forth as SEQ ID NO:46 (SQSTYVPLT) [mAb 5-2D7]; or
c. a VH comprising a HC CDR1 set forth as SEQ ID NO:19 (SYWMQ); a HC CDR2 SEQ ID NO:26 (EINPSNGRNNYNEKFKR); and a HC CDR3 SEQ ID NO:23 (SGLLDY); and/or a VL comprising a LC CDR1 set forth as SEQ ID NO:48 (RSSQSLVHSNGNTYLH); a LC CDR2 set forth as SEQ ID NO:44 (KVSNRFS); and a LC CDR3 set forth as SEQ ID NO:46 (SQSTYVPLT) [mAb 5-4A9]; or
d. a VH comprising a HC CDR1 set forth as SEQ ID NO:30 (SDYWT); a HC CDR2 SEQ ID NO:32 (YISYSGRTYYNPSLKS); and a HC CDR3 SEQ ID NO:34 (ERYYYGSLDY); and/or a VL comprising a LC CDR1 set forth as SEQ ID NO:48 (RSSQSLVHSNGNTYLH); a LC CDR2 set forth as SEQ ID NO:44 (KVSNRFS); and a LC CDR3 set forth as SEQ ID NO:53 (SQTTHVPPT) [mAb 5-9B12]; or
e. a VH region comprising a HC CDR1 set forth as SEQ ID No:30 (SDYWT); a HC CDR2 SEQ ID NO:37 (YISSSGRIYYNPSLKS); and a HC CDR3 SEQ ID NO:34 (ERYYYGSLDY); and/or a VL comprising a LC CDR1 set forth as SEQ ID NO:55 (RSSQGLVHSNGNTYLH); a LC CDR2 set forth as SEQ ID NO:44 (KVSNRFS); and a LC CDR3 set forth as SEQ ID NO:53 (SQTTHVPPT) [mAb 5-14A8]; or
f. a VH comprising a HC CDR1 set forth as SEQ ID NO:39 (SNWMN); a HC CDR2 SEQ ID NO:21 (EINPSNGRINYNEKFKS); and a HC CDR3 SEQ ID NO:23 (SGLLDY); and/or a VL comprising a LC CDR1 set forth as SEQ ID NO:48 (RSSQSLVHSNGNTYLH); a LC CDR2 set forth as SEQ ID NO:44 (KVSNRFS); and a LC CDR3 set forth as SEQ ID NO:56 (SQSTYLPLT) [mAb 5-14H5]; or
g. a variant thereof comprising a total of 1 or 2 mutations within any of the six CDRs; or
h. a VH comprising one or more heavy chain CDRs comprising at least 80%, 85%, 90%, 95%, 98%, 99% or 100% identity to any of SEQ ID NO:19, 21, 23, 26, 30, 32, 34, 37, or 39 and/or a VL comprising one or more light chain CDRs comprising at least 80%, 85%, 90%, 95%, 98%, 99% or 100% identity to any of SEQ ID NO:42, 44, 46, 48, 53, 55, or 56 [VpreB mAbs].

4. An isolated antibody, or antigen-binding fragment that specifically binds to the VpreB subunit of the SLC of human pre-BCR comprising:
a. a VH comprising a HC CDR1 set forth as SEQ ID NO:58 (SXWMX, wherein X at position 2 is Y or N and wherein X at position 5 is Q or N); a HC CDR2 set forth as SEQ ID NO:59 (EINPSNGRXNYNEKFKX, wherein X at position 9 is I or N and wherein X at position 17 is S or R); a HC CDR3 set forth as SEQ ID NO:23 (SGLLDY); and/or a VL comprising a LC CDR1 set forth as SEQ ID NO:60 (RSSQSLXHSNGNTYLH, wherein X at position 7 is I or V); a LC CDR2 set forth as SEQ ID NO:44 (KVSNRFS); and a LC CDR3 set forth as SEQ ID NO:61 (SQSTYXPLT, wherein X at position 6 is V or L) [VpreB consensus IA]; or
b. a VH comprising a HC CDR1 set forth as SEQ ID NO:30 (SDYWT); a HC CDR2 set forth as SEQ ID NO:62 (YISXSGRXYYNPSLKS, wherein X at position 4 is Y or S and wherein X at position 8 is T or I); a HC CDR3 set forth as SEQ ID NO:34 (ERYYYGSLDY); and/or a VL comprising a LC CDR1 set forth as SEQ ID NO:63 (RSSQXLVHSNGNTYLH, wherein X at position 5 is S or G); a LC CDR2 set forth as SEQ ID NO:44 (KVSNRFS); and a LC CDR3 set forth as SEQ ID NO:53 (SQTTHVPPT) [VpreB consensus IB].

5. An antibody or antigen-binding fragment of any of claims 3-4 wherein the antibody or antigen-binding fragment thereof comprises both the VL and the VH of any of claims 3-4.

6. An antibody or antigen-binding fragment thereof that binds to the same epitope of VpreB as any of the antibodies of claims 3-5.

7. An antibody or antigen-binding fragment thereof that cross-competes with any of the antibodies of claims 3-5 for binding to VpreB.

8. The antibody or antigen-binding fragment of any of claims 3-7 wherein the antibody or antigen-binding fragment thereof is a bispecific antibody that comprises a second VH, and optionally a second VL, that binds to a second antigen.

9. The antibody or antigen-binding fragment of any of claims 3-8 wherein the antibody or antigen-binding fragment thereof has an affinity for VpreB of about 10⁻⁷M or less.

10. An isolated antibody, or antigen-binding fragment that specifically binds to the lambda-5 subunit of the SLC of human pre-BCR that comprises:
a. a VH comprising a HC CDR1 set forth as SEQ ID NO: 79 (DYYLH); a HC CDR2 SEQ ID NO:81 (WIDPENGNTDYAPKFQG); and a HC CDR3 SEQ ID NO:83 (GYYDYDTDSAMDY); and/or a VL comprising a LC CDR1 set forth as SEQ ID NO:86 (RSSQSLVHSDGITYLH); a LC CDR2 set forth as SEQ ID NO:88 (KVSNRFS); and a LC CDR3 set forth as SEQ ID NO:90 (SQSTRVPWT) [mAb 4-15E6]; or
b. a VH comprising a HC CDR1 set forth as SEQ ID NO:115 (NYWMH); a HC CDR2 SEQ ID NO:123 (AIYPGNSDTSYNQKFKG); and a HC CDR3 SEQ ID NO:131 (ADYDGTPFDY); and/or (b) a VL comprising a LC CDR1 set forth as SEQ ID NO:143 (KSSQSLLDSDGETYLS); a LC CDR2 set forth as SEQ ID NO:154 (LVSKLDS); and a LC CDR3 set forth as SEQ ID NO:159 (WQGTHFPLT) [mAb 4-6D12]; or
c. a VH comprising a HC CDR1 set forth as SEQ ID NO:117 (SYWMH); a HC CDR2 SEQ ID NO:124 (AIYPGSSDTSYSQKFKG); and a HC CDR3 SEQ ID NO:133 (GDYDGTPFDY); and/or (b) a VL comprising a LC CDR1 set forth as SEQ ID NO:145 (KSGQSLLDSDGKTYLN); a LC CDR2 set forth as SEQ ID NO:156 (LVSKLHS); and a LC CDR3 set forth as SEQ ID NO:159 (WQGTHFPLT) [mAb 4-5G11]; or
d. a VH comprising a HC CDR1 set forth as SEQ ID NO:117 (SYWMH); a HC CDR2 SEQ ID NO:125 (AIYLGNTDTSYNQKFKG); and a HC CDR3 SEQ ID NO:131 (ADYDGTPFDY); and/or (b) a VL comprising a LC CDR1 set forth as SEQ ID NO:147 (RSSQSLLDSDGETYLS); a LC CDR2 set forth as SEQ ID NO:154 (LVSKLDS); and a LC CDR3 set forth as SEQ ID NO:159 (WQGTHFPLT) [mAb 4-7A6]; or
e. a VH comprising a HC CDR1 set forth as SEQ ID NO:117 (SYWMH); a HC CDR2 SEQ ID NO:123 (AIYPGNSDTSYNQKFKG); and a HC CDR3 SEQ ID NO:131 (ADYDGTPFDY); and/or (b) a VL comprising a LC CDR1 set forth as SEQ ID NO:143 (KSSQSLLDSDGETYLS); a LC CDR2 set forth as SEQ ID NO:154 (LVSKLDS); and a LC CDR3 set forth as SEQ ID NO:159 (WQGTHFPLT) [mAb 4-7C1]; or
f. a VH comprising a HC CDR1 set forth as SEQ ID NO:117 (SYWMH); a HC CDR2 SEQ ID NO:124 (AIYPGSSDTSYSQKFKG); and a HC CDR3 SEQ ID NO:133 (GDYDGTPFDY); and/or (b) a VL comprising a LC CDR1 set forth as SEQ ID NO:145 (KSGQSLLDSDGKTYLN); a LC CDR2 set forth as SEQ ID NO:156 (LVSKLHS); and a LC CDR3 set forth as SEQ ID NO:159 (WQGTHFPLT) [mAb 4-9H8]; or
g. a VH comprising a HC CDR1 set forth as SEQ ID NO:79 (DYYLH); a HC CDR2 SEQ ID NO:127 (WIDPENGATDYAPKFQG); and a HC CDR3 SEQ ID NO:137 (GYYDYDADSAMDY); and/or (b) a VL comprising a LC CDR1 set forth as SEQ ID NO:86 (RSSQSLVHSDGITYLH); a LC CDR2 set forth as SEQ ID NO:88 (KVSNRFS); and a LC CDR3 set forth as SEQ ID NO:160 (SQSARVPWT) [mAb 4-12G1]; or
h. a VH comprising a HC CDR1 set forth as SEQ ID NO:115 (NYWMH); a HC CDR2 SEQ ID NO:128 (AIYPGNSDTSYNQNFKG); and a HC CDR3 SEQ ID NO:131 (ADYDGTPFDY); and/or (b) a VL comprising a LC CDR1 set forth as SEQ ID NO:143 (KSSQSLLDSDGETYLS); a LC CDR2 set forth as SEQ ID NO:154 (LVSKLDS); and a LC CDR3 set forth as SEQ ID NO:159 (WQGTHFPLT) [mAb 4-17G9]; or
i. a VH comprising a HC CDR1 set forth as SEQ ID NO:115 (NYWMH); a HC CDR2 SEQ ID NO:129 (AVYPGNSDTSYSQKFTG); and a HC CDR3 SEQ ID NO:131 (ADYDGTPFDY); and/or (b) a VL comprising a LC CDR1 set forth as SEQ ID NO:143 (KSSQSLLDSDGETYLS); a LC CDR2 set forth as SEQ ID NO:154 (LVSKLDS); and a LC CDR3 set forth as SEQ ID NO:159 (WQGTHFPLT) [mAb 4-18G6]; or
j. a VH comprising a HC CDR1 set forth as SEQ ID NO:117 (SYWMH); a HC CDR2 SEQ ID NO:123 (AIYPGNSDTSYNQKFKG); and a HC CDR3 SEQ ID NO:131 (ADYDGTPFDY); and/or (b) a VL comprising a LC CDR1 set forth as SEQ ID NO:143 (KSSQSLLDSDGETYLS); a LC CDR2 set forth as SEQ ID NO:154 (LVSKLDS); and a LC CDR3 set forth as SEQ ID NO:159 (WQGTHFPLT) [mAb 4-19A9]; or
k. a VH comprising a HC CDR1 set forth as SEQ ID NO:117 (SYWMH); a HC CDR2 SEQ ID NO:123 (AIYPGNSDTSYNQKFKG); and a HC CDR3 SEQ ID NO:133 (GDYDGTPFDY); and/or (b) a VL comprising a LC CDR1 set forth as SEQ ID NO:152 (KSSQSLLDSDGETYLN); a LC CDR2 set forth as SEQ ID NO:157 (LASKLDS); and a LC CDR3 set forth as SEQ ID NO:159 (WQGTHFPLT) [mAb 4-20D2]; or
l. a variant thereof comprising a total of 1 or 2 mutations within any of the six CDRs; or
m. a VH comprising one or more heavy chain CDRs comprising at least 80%, 85%, 90%, 95%, 98%, 99% or 100% identity to SEQ ID NO:79, 81, 83, 115, 117, 123, 124, 125, 127, 128, 129, 131, 133 or 137, and/or a VL comprising one or more light chain CDRs comprising at least 80%, 85%, 90%, 95%, 98%, 99% or 100% identity to SEQ ID NO:86, 88, 90, 143, 145, 147, 152, 154, 156, 157, 159 or 160 [Lambda-5 mAbs].

11. An isolated antibody, or antigen-binding fragment that specifically binds to the lambda-5 subunit of the SLC of human pre-BCR that comprises:
a. a VH comprising a HC CDR1 set forth as SEQ ID NO:164 (XYWMH, wherein X at position 1 is N or S); a HC CDR2 set forth as SEQ ID NO:165 (AXYXGXXDTSYXQXFXG, wherein X at position 2 is I or V; wherein X at position 4 is P or L; wherein X at position 6 is N or S; wherein X at position 7 is S or T; wherein X at position 12 is N or S; wherein X at position 14 is K or N; and wherein X at position 16 is K or T); a HC CDR3 set forth as SEQ ID NO:166 (XDYDGTPFDY, wherein X at position 1 is A or G); and/or a VL comprising a LC CDR1 set forth as SEQ ID NO:167 (XSXQSLLDSDGXTYLX, wherein X at position 1 is K or R; wherein X at position 3 is S or G; wherein X at position 12 is E or K; and wherein X at position 16 is S or N); a LC CDR2 set forth as SEQ ID NO:168 (LXSKLXS, wherein X at position 2 is V or A; and wherein X at position 6 is D or H); and a LC CDR3 set forth as SEQ ID NO:159 (WQGTHFPLT) [Lambda-5 consensus IA]; or
b. a VH comprising a HC CDR1 set forth as SEQ ID NO:79 (DYYLH); a HC CDR2 set forth as SEQ ID NO:169 (WIDPENGXTDYAPKFQG, wherein X at position 8 is A or N); a HC CDR3 set forth as SEQ ID NO:170 (GYYDYDXDSAMDY, wherein X at position 7 is A or T); and/or a VL comprising a LC CDR1 set forth as SEQ ID NO:86 (RSSQSLVHSDGITYLH); a LC CDR2 set forth as SEQ ID NO:88 (KVSNRFS); and a LC CDR3 set forth as SEQ ID NO:171 (SQTXHVPPT, wherein X at position 4 is A or T) [Lambda-5 consensus IB].

12. An antibody or antigen-binding fragment of any of claims 3-4 wherein the antibody or antigen-binding fragment thereof comprises both the VL and the VH of any of claims 10-11.

13. An antibody or antigen-binding fragment thereof that binds to the same epitope of lambda-5 as any of the antibodies of claims 10-12.

14. An antibody or antigen-binding fragment thereof that cross-competes with any of the antibodies of claims 10-12 for binding to lambda-5.

15. The antibody or antigen-binding fragment of any of claims 10-14 where in the antibody or antigen-binding fragment thereof is a bispecific antibody that comprises a second VH that binds to a second antigen.

16. The antibody or antigen-binding fragment of any of claims 10-15 wherein the antibody or antigen-binding fragment thereof has an affinity for lambda-5 of about 10-7M or less,

17. The antibody or antigen-binding fragment of any of claims 1-16 wherein the antibody is a monoclonal antibody.

18. The antibody or antigen-binding fragment of any of claims 1-17 wherein the antibody or antigen-binding fragment thereof is conjugated to a cytotoxic drug moiety, optionally via an enzyme cleavable linker.

19. The antibody or antigen-binding fragment of any of claims 1-18 wherein the antibody is engineered for expression as a chimeric antigen receptor for expression in T cells or NK cells.

20. The antibody or antigen-binding fragment of any of claims 1-19 wherein the antibody or antigen-binding fragment thereof is internalized upon binding pre-BCR.

21. The antibody or antigen-binding fragment of any of claims 1-20 wherein the antibody is chimeric, human or humanized.

22. The antibody or antigen-binding fragment of any of claims 1-21 wherein the antibody is an IgG, or an IgG1, IgG2, IgG3 or IgG4.

23. An antigen-binding fragment of any of claims 1-21 wherein the antigen-binding fragment is a VL, VH, Fab, Fab′, F(ab′)2, scFv, or (scFv)2 fragment.

24. A nucleic acid encoding any of the antibody or antibody fragments of any of claims 1-23, or any of the VL or VH thereof.

25. An expression vector comprising the nucleic acid of claim 24, operably linked to a heterologous expression control sequence.

26. A host cell comprising the expression vector of claim 25.

27. A host cell comprising the nucleic acid of claim 24.

28. A T cell or NK cell comprising the nucleic acid of claim 24.

29. A method of making a recombinant antibody, or antigen-binding fragment thereof, comprising culturing the host cell of claim 26 or 27 in culture medium under conditions and for a time period suitable for expressing the antibody or antigen-binding fragment thereof of any of claims 1-23, and recovering the antibody or antigen-binding fragment from the host cell or culture medium.

30. A method of treating a subject with cancer or an autoimmune or immune-mediated inflammatory disease comprising administering a therapeutically effective amount of a monoclonal antibody or antigen-binding fragment thereof that specifically binds to cells expressing pre-BCR, preferably the antibody or antigen-binding fragment of any of claims 1-24.

31. The method of claim 30, wherein the antibody or antigen-binding fragment thereof specifically binds to the VpreB subunit of the SLC of human pre-BCR.

32. The method of claim 30, wherein the antibody or antigen-binding fragment thereof specifically binds to the lambda-5 subunit of the SLC of human pre-BCR.

33. The method of any of claims 30-32, wherein the subject has a cancer selected from the group consisting of acute lymphoblastic leukemia (ALL), acute myelogenous leukemia (AML), T-cell acute lymphoblastic leukemia (T-ALL), chronic lymphocytic leukemia (CLL), thymoma, lymphoma, mantel cell lymphoma (MCL), marginal zone lymphoma (MZL), diffuse large B cell lymphoma (DLBCL), follicular lymphoma (FL), Waldenstrom macroglobulinemia (WM), and multiple myeloma (MM).

34. The method of any of claims 30-33, further comprising administering a second therapeutic agent.

35. The method of claim 34, wherein the second therapeutic agent is a cytotoxic drug.

36. The method of any of claims 30-32, wherein said subject has an autoimmune or immune-mediated inflammatory disease selected from the group consisting of inflammatory bowel disease, ulcerative colitis, Crohn's disease, multiple sclerosis, psoriasis, rheumatoid arthritis, systemic lupus erythematosus, type 1 diabetes, vasculitis, asthma, eczema and atopic dermatitis, fibrosis, graft rejection, and graft-versus-host-disease.

37. The method of claim 36, further comprising administering a second therapeutic agent.

38. A method of treating a leukemia or lymphoma in a companion animal, such as a dog or a cat, comprising administering a therapeutically effective amount of a monoclonal antibody or antigen-binding fragment thereof that specifically binds to cells expressing pre-BCR.

39. The method of claim 38 wherein the antibody or antigen-binding fragment thereof specifically binds to the VpreB subunit of the SLC of the pre-BCR.

40. The method of claim 38 wherein the antibody or antigen-binding fragment thereof specifically binds to the lambda-5 subunit of the SLC of the pre-BCR.

41. An in vitro diagnostic method for the diagnosis of a disease or condition in claim 33 or 36, comprising contacting an antibody, or antigen-binding fragment thereof, according to any of claims 1-23 with a sample from a subject known or suspected to be afflicted with said disease or condition.

42. A diagnostic kit, comprising the antibody, or antigen-binding fragment thereof, as defined in any of claims 1-23, and instructions for use, and, optionally, a biologically active sub stance.
